(19) United States
(12) Patent Application Publication
(10) Pub. No.: US 2023/0372472 A1
(43) Pub. Date: Nov. 23, 2023

(54) IMMUNOTHERAPEUTIC VIRUS FOR THE TREATMENT OF CANCER

(71) Applicants: PRESIDENT AND FELLOWS OF HARVARD COLLEGE, Cambridge, MA (US); DANA-FARBER CANCER INSTITUTE, INC., Boston, MA (US)

(72) Inventors: David M. KNIPE, Cambridge, MA (US); Michael J. WALSH, Cambridge, MA (US); Stephanie K. DOUGAN, Cambridge, MA (US)

(73) Assignees: PRESIDENT AND FELLOWS OF HARVARD COLLEGE, Cambridge, MA (US); DANA-FARBER CENTER INSTITUTE, INC., Boston, MA (US)

(21) Appl. No.: 18/028,139

(22) PCT Filed: Sep. 24, 2021
(86) PCT No.: PCT/US2021/051956

§ 371 Date: Mar. 23, 2023

Related U.S. Application Data
(60) Provisional application No. 63/083,487, filed on Sep. 25, 2020.

(51) Int. Cl. A61K 39/245; C12N 7/00; A61K 35/763; A61K 38/20; A61P 35/00

(52) U.S. Cl. CPC A61K 39/245; C12N 7/00; A61K 35/763; A61K 38/208; A61P 35/00; C12N 2710/16621; C12N 2710/16632; C12N 2710/16671; A61K 2039/572

(57) ABSTRACT

Provided herein is a replication-defective oncolytic herpes simplex virus 1 (HSV-1) recombinant virus, comprising within its genome: a coding sequence encoding a fusion protein.

### CROSS-REFERENCE TO RELATED APPLICATIONS

This application is a 35 U.S.C. § 371 National Stage Entry Application of International Application No. PCT/US2021/051956 filed Sep. 24, 2021, which designated the U.S., and which claims benefit under 35 U.S.C. § 119(e) of U.S. Provisional Application No. 63/083,487 filed Sep. 25, 2020, the contents of which are incorporated herein by reference in their entireties.

### GOVERNMENT SUPPORT

This invention was made with government support under grant no. AI 098681 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Oct. 29, 2021, is named 002806-098100WOPT SL.txt and is 106,975 bytes in size.

### TECHNICAL FIELD

The technology described herein relates to compositions and methods for treating cancer and uses thereof.

### BACKGROUND

Cancer is an often fatal disease caused by abnormal cell growth that invades, spreads, or metastasizes, throughout the body. Immunotherapies, such as oncolytic viral therapy, have emerged as a breakthrough treatment for cancer that harnesses a virus to reproduce within host cells that induces an immunogenic response preferentially directed toward lysing cancerous cells. However, some viral immunotherapies have serious side effects or can be largely ineffective for subsets of patients. Thus, there is a need for viral therapies that are safe and effective for a variety of tumor types.

### SUMMARY

The compositions and methods provided herein, relate in part, to the discovery of a non-replicating HSV virus, that can serve as a safe immunotherapeutic and vector virus for the treatment of a variety of tumor types. The recombinant virus provided herein can deliver cytokines (e.g., IL-12), checkpoint inhibitors, or tumor-associated antigens to a tumor site in a subject to elicit an immune response by the host that results in tumor cell killing.

In one aspect, provided herein is a replication-defective oncolytic herpes simplex virus 1 (HSV-1) recombinant virus, comprising within its genome: at least one therapeutic gene coding sequence. Generally, the recombinant virus genome does not encode a functional ICP4, ICP22, ICP27 and/or ICP47 protein. For example, the genome comprises at least one alteration in a gene encoding infected cell polypeptides (ICP) 4, a gene encoding ICP22, a gene encoding ICP27 and/or a gene encoding ICP47. Further, the therapeutic gene coding sequence may or may not comprise an internal ribosome entry site (IRES).

Generally, the therapeutic gene coding sequence can encode for a protein. For example, the protein can be a fusion protein. Alternatively, the protein can be a non-fusion protein. In some embodiments of the various aspects, the therapeutic gene coding sequence encodes a fusion protein comprising a first domain and a second domain linked via a linker. In some embodiments of the various aspects, the nucleotide sequence encoding the linker does not comprise an IRES.

In some embodiments of the various aspects, the therapeutic gene coding sequence encodes a cytokine. For example, the therapeutic gene coding sequence encodes a heterodimeric cytokine. In some embodiments of the various aspects, the therapeutic gene coding sequences encodes IL-12. For example, the therapeutic gene coding sequences encodes a fusion protein comprising a p40 subunit of IL-12 and a p34 subunit of IL-12 as separate domains. The p40 and p35 domains can be linked via a linker.

In another aspect, provided herein is a composition comprising the recombinant virus provided herein. For example, the composition can be a pharmaceutical composition comprising the recombinant virus and a pharmaceutically acceptable carrier or excipient.

In still another aspect, provided herein is a vaccine and/or immunomodulatory virus comprising the recombinant virus provided herein. In some embodiments, vaccine can further comprise an adjuvant.

In yet another aspect, provided herein is a method of eliciting and/or modifying an immune response in a subject. Generally, the method comprises administering to a subject in need thereof the recombinant virus provided herein. In some embodiments, the method can further comprise a step of selecting a subject for eliciting and/or modifying an immune response.

In still yet another aspect, provided herein is a method of treating cancer. Generally, the method comprises administering to a subject in need thereof the recombinant virus provided herein.

### BRIEF DESCRIPTION OF THE DRAWINGS

This application file contains at least one drawing executed in color. Copies of this patent application publication with color drawings will be provided by the Office upon request and payment of the necessary fee.

FIGS. 1A-1D show that an exemplary replication-defective oncolytic Herpes simplex virus 1 (HSV-1) recombinant virus (d106S-IL12) described herein suppresses internal interferon response, robustly secretes IL-12, and boosts external interferon response.

FIGS. 2A-2D show that injection of d106S-IL-12 into B16 tumors initially increases infiltration of CD8 cells.

FIGS. 3A-3D show that injection of d106S-IL12 into B16 tumors results in mass infiltration of CD45+ cells and tumor-antigen specific CD8 T cells.

FIGS. 4A-4D show that intratumoral injection of d106S-IL12 improves survival response to B16 melanoma better than recombinant IL12.

FIGS. 5A-5D show that checkpoint blockade does not enhance d106S-IL12 survival benefit.

FIGS. 6A-6F show pancreatic cancer cells are susceptible to d106S-IL12 induced equilibrium that is independent of CD8+ T cell recognition. (FIG. 6A) Murine C2 pancreatic cancer cells were infected in vitro at a multiplicity of infection (MOI) 0, 1, 10, and 50 with d106S-GFP. Percent GFP-positive cells were determined at 24 hours post infection by flow cytometry. (FIG. 6B) 2×105 C2 cells were implanted subcutaneously in flanks of C57BL/6 mice and treatment with PBS or d106S-IL12 (N=5 mice per group) began on day seven (as indicated by arrow) and proceeded every three days. (FIG. 6C) Individual growth curves of mice from (FIG. 6B). (FIG. 6D) C2WT and C2βm−/− cells were cultured for 24 hours with or without IFNγ (50 ng/ml) and H2-Kb/Db (MHC-I) expression was measured by flow cytometry (MFI; mean fluorescence intensity). (FIG. 6E) C2WT or (FIG. 6F) C2βm−/− cells were implanted subcutaneously in flanks of mice and PBS, d106S, or d106S-IL12 treatment began on day seven and proceeded every three days (N=5 mice per group for each tumor type). Mice were sacrificed if tumors ulcerated or reached 1000 mm3. Values are mean±SEM. Survival groups were compared with log-rank test; **P<0.01.

FIGS. 7A-7F show early response to type I IFN is important, but equilibrium is maintained by IFNγ, and CD8+ and CD4+ T cells provide redundant protection. (FIG. 7A) Mice were challenged with 5×105 B16 cells in the flank and intratumoral treatment with PBS or d106S-IL12 began on day seven and proceeded every three days. Some mice were also treated intratumorally with anti-IFNAR blocking antibodies (100 μg) every three days (N=5 for PBS, 10 for d106S-IL12), while the rest of the mice received no blockade (N=62). (FIG. 7B) Representative timepoint showing tumor size at day 22 in the middle of anti-IFNAR treatment. (FIG. 7C) Survival curve up to day 40. Arrow indicates start treatment; purple box indicates duration of anti-IFNAR treatment. (FIG. 7D) At day 40, surviving mice were rerandomized to new treatment groups and injected intraperitoneally with either isotype control, anti-CD4, anti-CD8, anti-IFNγ, or anti-NK1.1 depleting antibodies (100 μg) every three days while also continuing to receive d106S-IL12 treatment (curves go down in size as the rerandomized groups contain only remaining live mice, while left-side curves account for endpoint tumors). (FIG. 7E) Survival curve including rerandomization. (FIG. 7F) Individual growth curves of each treatment group (N=11 isotype, N=12 anti-CD4, N=12 anti-CD8, N=12 anti-IFNγ, N=11 anti-NK1.1). Dashed line indicates the start of depletion. Mice were sacrificed if tumors ulcerated or reached 2000 mm3. Values are mean±SEM. Survival groups were compared with Bonferroni-corrected log-rank test; *P<0.05.

FIGS. 8A-8E show antibodies successfully deplete target cell types. Remaining mice from FIGS. 7A-7F were bled retrorbitally at day 62 and peripheral blood stained to confirm the depletion of (FIG. 8A, 8B) CD4+ T cells and (FIG. 8A, 8C) CD8+ T cells by flow cytometry. (FIG. 8D, 8E) At day 63, these mice were sacrificed and their spleens stained to confirm depletion of NK cells by flow cytometry. Representative gating shown. Values are mean ±SEM. NK cells were compared using an unpaired t-test; ***P<0.001.

FIG. 9A-9D show IFNγ secretion is more important than presence of α/β T cells for response to d106S-IL12. (FIG. 9A) Wild-type, (FIG. 9B) IFNγ−/−, or (FIG. 9C) TCRα−/− mice were challenged with B16 tumors and treated with PBS (N=5 per mouse strain) or d106S-IL12 (N=10, 10, and 8, respectively) starting at day seven and proceeding every three days. (FIG. 9D) Tumor volume and survival of d106S-IL12 groups. Values are mean±SEM. Survival groups were compared with Bonferroni-corrected log-rank test; *P<0.05, **P<0.01, ***P<0.001, ****P<0.0001.

FIG. 10A and 10B show confirmation that TCRα−/− mice lack CD4+ and CD8+ T cells. Mice sacrificed during the experiment from FIG. 9A-9D had spleens harvested and stained to confirm the absence of (FIG. 10A) CD4+ and (FIG. 10B) CD8+ T cells. Representative gating shown. Values are mean±SEM. Cell amounts were compared using an unpaired t-test; ***P<0.001.

FIG. 11A-11D show γδ T cells, cross-presenting DCs and perforin/granzyme are unnecessary for establishment of equilibrium by d106S-IL12. (FIG. 11A) Wild-type, (FIG. 11B) TCRδ−/−, (FIG. 11C) BATF3−/−, or (FIG. 11D) Prfl−/− mice were challenged with B16 tumors and treated with PBS (N=5 for wild-type and N=4 for knockout mice) or d106S-IL12 (N=10, 4, 6, and 5, respectively) starting at day seven and proceeding every three days. Values are mean ±SEM.

FIG. 12A-12J show tumors lacking antigen presentation or IFN response can still respond to d106S-IL12 therapy. CRISPR-Cas9 gene edited was used to generate (32m, STAT1, and IFNAR knockout B16 cell lines. These cell lines were transduced with a nectin-1 encoding lentivirus to generate cell lines permissive to HSV entry. These B16 knockout, nectin-1+ cells were compared to the original wild-type, nectin-1+ cells used in all prior experiments. (FIG. 12A) Cells were infected at an MOI 0, 1, 10, and 25 with d106S-GFP and percent GFP-positive cells were determined at 24 hours post infection by flow cytometry. (FIG. 12B) Cells were plated and confluence determined every 24 hours by Celigo image cytometer. Cells were cultured in vitro with (FIG. 12C) 50 ng/ml IFNγ or (FIG. 12D) 100 ng/ml IFNα and H2-Kb/Db (MHC-I) expression determined by flow cytometry 24 hours later. Wild-type mice were challenged with either (FIG. 12E) wild-type, (FIG. 12F) β2m−/−, (FIG. 12G) STAT1−/−, or (FIG. 12H) IFNAR−/− B16(Nectin1+) tumors and treated with PBS or d106S-IL12 starting at day seven and proceeding every three days. (FIG. 12I) Wild-type or (FIG. 12J) STAT1−/− B16 cells were plated with varying concentrations of IFNγ and confluence determined every 24 hours by Celigo image cytometer. Values are mean±SEM, N=3 unless otherwise noted. Confluence was compared by two-way ANOVA with Dunnett's multiple comparisons test, *P<0.05, ***P<0.001.

### DETAILED DESCRIPTION

Oncolytic viruses are genetically modified viruses that preferentially replicate in host cancer cells, leading to the production of new viruses and ultimately, cell death. Herpes simplex virus (HSV) possesses several unique properties as an oncolytic agent. It can infect a broad range of cell types and has a short replication cycle (9 to 18 h). The use of a replication-conditional strain of HSV-1 as an oncolytic agent was first reported for the treatment of malignant gliomas. Since then, various efforts have been made in an attempt to broaden their therapeutic efficacy and increase the replication specificity of the virus in tumor cells. Not surprisingly, however, deletion of genes that impair viral replication in normal cells also leads to a marked decrease in the oncolytic activity of the virus for the targeted tumor cells. Currently, no oncolytic viruses that are able to kill only tumor cells while leaving normal cells intact are available. Consequently, the therapeutic doses of existing oncolytic viruses are significantly restricted. The availability of an oncolytic virus whose replication can be tightly controlled and adjusted pharmacologically would offer greatly increased safety and therapeutic efficacy. Such a regulatable oncolytic virus would minimize the risk of uncontrolled replication in adjacent and distant tissues as well as undesirable progeny virus overload in the target area after the tumor has been eliminated. This regulatory feature would also allow the oncolytic activity of the virus to be quickly shut down should adverse effects be detected.

The compositions and methods provided herein, relate in part, to the discovery of a non-replicating HSV virus, termed d106S, which can serve as a safe immunotherapeutic and vector virus for the treatment of a broad spectrum of tumor types. This recombinant virus allows for expression of transgenes that can encode for immunostimulatory molecules, such as cytokines (e.g., IL-12), checkpoint inhibitors, or tumor-associated antigens. The working examples provided herein demonstrate that interleukin (IL)-12 can be delivered to the tumor microenvironment via the d106S HSV that drives CD8 T cell infiltration into the tumor and subsequently improves the survival of animals with cancer.

IL-12 is a potent cytokine capable of organizing a Thl response against tumors by enhancing the growth and cytotoxicity of NK cells, CD8+ and CD4+ T cells. However, due to the pleiotropic effects of IL-12, dose-limiting toxicities often become a barrier to effective treatment. The replication-defective d106S virus provided herein releases a large burst of IL-12 locally within the tumor environment. Because the virus cannot replicate, further production of IL-12 would only be dependent on additional injections of the virus. The d106S-IL12 vector provided herein is capable of directing CD8 infiltration into B16 tumors and treating cancer.

### Oncolytic Virus Compositions

In one aspect, the methods and compositions provided herein comprise a replication-defective oncolytic Herpes simplex virus 1 recombinant virus.

Herpesviruses are enveloped double stranded DNA-containing viruses in an icosahedral nucleocapsid. HSV double-stranded, linear DNA genome is comprised of 152 kb of nucleotide sequence, which encodes about 80 genes. At least seven herpesviruses are associated with infection in humans, including herpes simplex virus type-1 (HSV-1), herpes simplex virus type-2 (HSV-2), varicella zoster virus (VZV), Epstein Barr virus (EBV), cytomegalovirus (CMV), human herpesvirus-6 (HHV-6) and human herpesvirus-7 (HHV-7).

HSV-1 exhibits a pattern of gene expression during productive infection of a host cell or subject that is stringently regulated (see, e.g., Fields et al. Virology, 1990, Raven Press, NY; DeLuca et al., J. Virol., 56:558 (1985); McCarthy et al., J. Virol., 63:18 (1989); Gao et al., J. Virol. 63:5258 (1989); and Quinlan et al., Cell, 36:657 (1984) which are incorporated herein by reference in their entireties). The viral genes are transcribed by cellular RNA polymerase II and are temporally regulated, resulting in the transcription and subsequent synthesis of gene products in roughly discernible phases: Immediate Early (IE, or α), Early (E, or β) and Late (L, or γ). Immediately following the arrival of an HSV genome into the nucleus of an infected cell, the IE genes are transcribed. The IE genes are all activated by a complex including the HSV virion particle VP16 and the cellular factor, Oct-1, which binds to a consensus sequence (TAATGARAT) regulating IE gene expression (see, e.g., Preston et al., Cell, 52, 425-35 (1988), which is incorporated herein by reference in its entirety). The presence of this sequence, thus, confers the IE quality to HSV regulatory sequences. The efficient expression of IE genes, thus, does not require prior viral protein synthesis, while later expression depends upon the presence of IE gene products. The products of IE genes are required to activate transcription and regulate the remainder of the viral genome.

Infected Cell Peptide 4 (ICP4), ICP0, ICP27, ICP22, and ICP47 are the immediate early gene products, and these show varying degrees of essentiality to HSV function. These phosphoproteins possess regulatory activities that can prime the host cell for the efficient cascade of subsequent viral gene expression, DNA replication, and the production of progeny virions. For example, ICP0 activates most test promoters in transient assays (Quinlan and Knipe, Mol. Cell. Biol., 5, 957-63 (1985)), elevates levels of viral gene expression and growth in tissue culture and in the trigeminal ganglia (Cai and Schaffer, J. Virol., 66, 2904-15 (1992)), and facilitates the reactivation of virus from latency (Leib, et al., J. Virol., 63, 759-68 (1989)). ICP27 modulates the activity of ICP4 and ICP0, and it regulates viral and cellular mRNA processing. These activities of ICP27 contribute to efficient DNA replication, hence it is necessary for viral growth; however, ICP27 also regulates the proper expression of early and late genes. ICP22 promotes efficient late gene expression in a cell-type dependent manner and is involved in the production of a novel modified form of RNA Pol II. ICP4 is a large multifunctional protein that can act as a transcription factor that either represses or activates transcription through contacts with the general transcriptional machinery. ICP4 controls both virus infectivity and the transition from IE to later transcription.

The recombinant virus provided herein and demonstrated in the working examples has been engineered as a gene expression vector for transient expression of a trans-gene (e.g., IL-12) and also provides immunotherapeutic effects that lead to tumor cell killing. See, e.g., Liu X, et al. Vaccine. 2009;27:2760-2767; Thomann S, et al. Immunology. 2015:327-338, which is incorporated herein by reference in its entirety.

In some embodiments of any of the aspects, the recombinant virus genome provided herein comprises at least one alteration in one or more genes encoding infected cell polypeptides (ICPs). In some embodiments of any of the aspects, the genome comprises at least one alteration in each of a gene encoding infected cell polypeptides (ICP) 4, a gene encoding ICP22, a gene encoding ICP27 and a gene encoding ICP47. In some embodiments of any of the aspects, the genome does not encode a functional ICP4, ICP22, ICP27 and/or ICP47 proteins. Non-limiting examples of HHV-1 ICP amino acid and gene sequences are provided in TABLE 1.

In some embodiments of any of the aspects, the genome comprises at least one alteration in ICP4. In some embodiments of any of the aspects, the genome comprises at least one alteration in ICP22. In some embodiments of any of the aspects, the genome comprises at least one alteration in ICP27. In some embodiments of any of the aspects, the genome comprises at least one alteration in ICP47. In some embodiments of any of the aspects the genome comprises at least one alteration in ICP4 and ICP22. In some embodiments of any of the aspects, the genome comprises at least one alteration in ICP4 and ICP27. In some embodiments of any of the aspects, the genome comprises at least one alteration in ICP4 and ICP47. In some embodiments of any of the aspects, the genome comprises at least one alteration in ICP22 and ICP27. In some embodiments of any of the aspects, the genome comprises at least one alteration in ICP22 and ICP47. In some embodiments of any of the aspects, the genome comprises at least one alteration in ICP27 and ICP47. In some embodiments of any of the aspects, the genome comprises at least one alteration in ICP4, ICP22, and ICP27. In some embodiments of any of the aspects, the genome comprises at least one alteration in ICP4, ICP22, and ICP47. In some embodiments of any of the aspects, the genome comprises at least one alteration in ICP22, ICP27, and ICP47.

In some embodiments, the genome comprises at least one, two, three, four, five, six, seven, eight, nine, ten, or more alterations in the genes encoding an ICP polypeptide of HHV-1. In some embodiments of any of the aspects, the genome comprises at least one alteration in two genes encoding infected cell polypeptides (ICP) 4, a gene encoding ICP22, a gene encoding ICP27, and/or a gene encoding ICP47. In some embodiments of any of the aspects, the genome comprises at least one alteration in three genes encoding infected cell polypeptides (ICP) 4, a gene encoding ICP22, a gene encoding ICP27, and/or a gene encoding ICP47. In some embodiments of any of the aspects, the genome comprises at least one alteration in all four of the genes encoding infected cell polypeptides (ICP) 4, a gene encoding ICP22, a gene encoding ICP27, and a gene encoding ICP47.

In some embodiments of any of the aspects, the genome does not encode a functional ICP4 protein. In some embodiments of any of the aspects, the genome does not encode a functional ICP22 protein. In some embodiments of any of the aspects, the genome does not encode a functional ICP27 protein. In some embodiments of any of the aspects, the genome does not encode a functional ICP47 protein. In some embodiments of any of the aspects, the genome does not encode a functional ICP4 protein or an ICP22 protein. In some embodiments of any of the aspects, the genome does not encode a functional ICP4 protein or an ICP27 protein. In some embodiments of any of the aspects, the genome does not encode a functional ICP4 protein or an ICP47 protein. In some embodiments of any of the aspects, the genome does not encode a functional ICP22 protein or an ICP27 protein. In some embodiments of any of the aspects, the genome does not encode a functional ICP22 protein or an ICP47 protein. In some embodiments of any of the aspects, the genome does not encode a functional ICP27 protein or an ICP47 protein.

In some embodiments of any of the aspects, the genome further comprises at least one alteration in a gene encoding ICPO. ICPO plays a major role in enhancing the reactivation of HSV from latency and confers a significant growth advantage on the virus at low multiplicities of infection. Sequences for the HSV ICP0 and ICP4 promoters and for the genes whose regulation they endogenously control are well known in the art (e.g., SEQ ID NO: 1, TABLE 1). For example, see McGeoch et al., J. Gen. Virol. 72:3057-3075 (1991); McGeoch et al, Nucl. Acid Res. 77:1727-1745 (1986); Perry, et al., J. Gen. Virol. 67:2365-2380 (1986)) and procedures for making viral vectors containing these elements have been previously described (see, e.g., US 2005/0266564). These promoters are not only very active in promoting gene expression, they are also specifically induced by VP 16, a virus-associated transactivator released when HSV-1 or HSV-2 infects a cell.

TABLE 1
Exemplary Wild-type HHV-1 and HHV2 ICP gene and amino acid sequences

| Gene/ | | |
|---|---|---|
| Polypeptide | | SEQ ID NO: |
| name | Sequence | Accession # |
| ICP0 | MEPRPGASTRRPEGRPQREPAPDVWVFPCDRDLPDSSDSEAETEVGGRGDAD | SEQ ID NO: 1 |
| polypeptide | HHDDDSASEADSTDTELFETGLLGPQGVDGGAVSGGSPPREEDPGSCGGAPP | UniProtKB |
| | REDGGSDEGDVCAVCTDEIAPHLRCDTFPCMHRFCIPCMKTWMQLRNTCPLC | P08393 |
| | NAKLVYLIVGVTPSGSFSTIPIVNDPQTRMEAEEAVRAGTAVDFIWTGNQRF | (ICP0_HHV11) |
| | APRYLTLGGHTVRALSPTHPEPTTDEDDDDLDDADYVPPAPRRTPRAPPRRG | |
| | AAAPPVTGGASHAAPQPAAARTAPPSAPIGPHGSSNTNTTTNSSGGGGSRQS | |
| | RAAAPRGASGPSGGVGVGVGVVEAEAGRPRGRTGPLVNRPAPLANNRDPIVI | |
| | SDSPPASPHRPPAAPMPGSAPRPGPPASAAASGPARPRAAVAPCVRAPPPGP | |
| | GPRAPAPGAEPAARPADARRVPQSHSSLAQAANQEQSLCRARATVARGSGGP | |
| | GVEGGHGPSRGAAPSGAAPLPSAASVEQEAAVRPRKRRGSGQENPSPQSTRP | |
| | PLAPAGAKRAATHPPSDSGPGGRGQGGPGTPLTSSAASASSSSASSSSAPTP | |
| | AGAASSAAGAASSSASASSGGAVGALGGRQEETSLGPRAASGPRGPRKCARK | |
| | TRHAETSGAVPAGGLTRYLPISGVSSVVALSPYVNKTITGDCLPILDMETGN | |
| | IGAYVVLVDQTGNMATRLRAAVPGWSRRTLLPETAGNHVMPPEYPTAPASEW | |
| | NSLWMTPVGNMLFDQGTLVGALDFRSLRSRHPWSGEQGASTRDEGKQ | |
| ICP0 gene | >JQ673480.1: 2235-5467 Human herpesvirus 1 strain | SEQ ID NO: 2 |
| sequence | KOS, complete genome RL2 | JQ673480.1: |
| | ATGGAGCCCCGCCCCGGAGCGAGTACCCGCCGGCCTGAGGGCCGCCCCCAGC | 2235-5467 |
| | GCGAGGTGAGGGGCCGGGCGCCATGTCTGGGGCGCCATGTTGGGGGGCGCCA | |
| | TGTTGGGGGGCGCCATGTTGGGGGACCCCCGACCCTTACACTGGAACCGGCC | |
| | GCCATGTTGGGGGACCCCCACTCATACACGGGAGCCGGGCGCCATGTTGGGG | |
| | CGCCATGTTAGGGGGCGTGGAACCCCGTGACACTATATATACAGGGACCGGG | |
| | GGCGCCATGTTAGGGGGCGCGGAACCCCCTGACCCTATATATACAGGGACCG | |
| | GGGTCGCCCTGTTAGGGGTCGCCATGTGACCCCCTGACTTTATATATACAGA | |
| | CCCCCAACACCTACACATGGCCCCTTTGACTCAGACGCAGGGCCCGGGGTCG | |
| | CCGTGGGACCCCCCTGACTCATACACAGAGACACGCCCCCACAACAAACACA | |
| | CAGGGACCGGGGTCGCCGTGTTAGGGGGCGTGGTCCCCACTGACTCATACGC | |
| | AGGGCCCCCTTACTCACACGCATCTAGGGGGGTGGGGAGGAGCCGCCCGCCA | |
| | TATTTGGGGGACGCCGTGGGACCCCCGACTCCGGTGCGTCTGGAGGGCGGGA | |
| | GAAGAGGGAAGAAGAGGGGTCGGGATCCAAAGGACGGACCCAGACCACCTTT | |
| | GGTTGCAGACCCCTTTCTCCCCCCTCTTCCGAGGCCAGCAGGGGGGCAGGAC | |
| | TTTGTGAGGCGGGGGGGGAGGGGGAACTCGTGGGCGCTGATTGACGCGGGAA | |
| | ATCCCCCCATTCTTACCCGCCCCCCCTTTTTTCCCCTCAGCCCGCCCCGGAT | |
| | GTCTGGGTGTTTCCCTGCGACCGAGACCTGCCGGACAGCAGCGACTCGGAGG | |
| | CGGAGACCGAAGTGGGGGGGCGGGGGGACGCCGACCACCATGACGACGACTC | |
| | CGCCTCCGAGGCGGACAGCACGGACACGGAACTGTTCGAGACGGGGCTGCTG | |
| | GGGCCGCAGGGCGTGGATGGGGGGGCGGTCTCGGGGGGGAGCCCCCCCCGCG | |
| | AGGAAGACCCCGGCAGTTGCGGGGGCGCCCCCCCTCGAGAGGACGGGGGGAG | |
| | CGACGAGGGCGACGTGTGCGCCGTGTGCACGGATGAGATCGCGCCCCACCTG | |
| | CGCTGCGACACCTTCCCGTGCATGCACCGCTTCTGCATCCCGTGCATGAAAA | |
| | CCTGGATGCAATTGCGCAACACCTGCCCGCTGTGCAACGCCAAGCTGGTGTA | |
| | CCTGATAGTGGGCGTGACGCCCAGCGGGTCGTTCAGCACCATCCCGATCGTG | |
| | AACGACCCCCAGACCCGCATGGAGGCCGAGGAGGCCGTCAGGGCGGGCACGG | |
| | CCGTGGACTTTATCTGGACGGGCAATCAGCGGTTCGCCCCGCGGTACCTGAC | |
| | CCTGGGGGGGCACACGGTGAGGGCCCTGTCGCCCACCCACCCTGAGCCCACC | |
| | ACGGACGAGGATGACGACGACCTGGACGACGGTGAGGCGGGGGGGCGGCGAG | |
| | GACCCTGGGGGAGGAGGAGGAGGGGGGGGGGAGGGAGGAATAGGCGGGCGGG | |
| | CGGGCGAGGAAAGGGCGGGCCGGGGAGGGGGCGTAACCTGATCGCGCCCCCC | |
| | GTTGTCTCTTGCAGCAGACTACGTACCGCCCGCCCCCCGCCGGACGCCCCGC | |
| | GCCCCCCCACGCAGAGGCGCCGCCGCGCCCCCCGTGACGGGCGGGGCGTCTC | |
| | ACGCAGCCCCCCAGCCGGCCGCGGCTCGGACAGCGCCCCCCTCGGCGCCCAT | |
| | CGGGCCACACGGCAGCAGTAACACTAACACCACCACCAACAGCAGCGGCGGC | |
| | GGCGGCTCCCGCCAGTCGCGAGCCGCGGTGCCGCGGGGGGCGTCTGGCCCCT | |
| | CCGGGGGGGTTGGGGTTGTTGAAGCGGAGGCGGGGCGGCCGAGGGGCCGGAC | |
| | GGGCCCCCTTGTCAACAGACCCGCCCCCCTTGCAAACAACAGAGACCCCATA | |
| | GTGATCAGCGACTCCCCCCCGGCCTCTCCCCACAGGCCCCCCGCGGCGCCCA | |
| | TGCCAGGCTCCGCCCCCCGCCCCGGTCCCCCCGCGTCCGCGGCCGCGTCGGG | |
| | CCCCGCGCGCCCCCGCGCGGCCGTGGCCCCGTGTGTGCGGGCGCCGCCTCCG | |
| | GGGCCCGGCCCCCGCGCCCCGGCCCCCGGGGCGGAGCCGGCCGCCCGCCCCG | |
| | CGGACGCGCGCCGTGTGCCCCAGTCGCACTCGTCCCTGGCTCAGGCCGCGAA | |
| | CCAAGAACAGAGTCTGTGCCGGGCGCGTGCGACGGTGGCGCGCGGCTCGGGG | |
| | GGGCCGGGCGTGGAGGGTGGACACGGGCCCTCCCGCGGCGCCGCCCCCTCCG | |
| | GCGCCGCCCCCTCCGGCGCCCCCCCGCTCCCCTCCGCCGCCTCTGTCGAGCA | |
| | GGAGGCGGCGGTGCGTCCGAGGAAGAGGCGCGGGTCGGGCCAGGAAAACCCC | |
| | TCCCCCCAGTCCACGCGTCCCCCCCTCGCGCCGGCAGGGGCCAAGAGGGCGG | |
| | CGACGCACCCCCCCTCCGACTCAGGGCCGGGGGGGCGCGGCCAGGGAGGGCC | |
| | CGGGACCCCCCTGACGTCCTCGGCGGCCTCCGCCTCTTCCTCCTCCGCCTCT | |
| | TCCTCCTCGGCCCCGACTCCCGCGGGGGCCACCTCTTCCGCCACCGGGGCCG | |
| | CGTCCTCCTCCGCTTCCGCCTCCTCGGGCGGGGCCGTCGGTGCCCTGGGAGG | |
| | GAGACAAGAGGAAACCTCCCTCGGCCCCCGCGCTGCTTCTGGGCCGCGGGGG | |
| | CCGAGGAAGTGTGCCCGGAAGACGCGCCACGCGGAGACTTCCGGGGCCGTCC | |
| | CCGCGGGCGGCCTCACGCGCTACCTGCCCATCTCGGGGGTCTCTAGCGTGGT | |
| | CGCCCTGTCGCCTTACGTGAACAAGACGATCACGGGGGACTGCCTGCCCATC | |
| | CTGGACATGGAGACGGGGAACATCGGGGCGTACGTGGTCCTGGTGGACCAGA | |
| | CGGGAAACATGGCGACCCGGCTGCGGGCCGCGGTCCCCGGCTGGAGCCGCCG | |
| | CACCCTGCTCCCCGAGACCGCGGGTAACCACGTGACGCCCCCCGAGTACCCG | |
| | ACGGCCCCCGCGTCGGAGTGGAACAGCCTCTGGATGACCCCCGTGGGGAACA | |
| | TGCTGTTCGACCAGGGCACCCTAGTGGGCGCCCTGGACTTCCGCAGCCTGCG | |
| | GTCTCGGCACCCGTGGTCCGGGGAGCAGGGGGCGTCGACCCGGGACGAGGGA | |
| | AAACAATAA | |
| ICP4 | MASENKQRPGSPGPTDGPPPTPSPDRDERGALGWGAETEEGGDDPDHDPDHP | SEQ ID NO: 3 |
| | HDLDDARRDGRAPAAGTDAGEDAGDAVSPRQLALLASMVEEAVRTIPTPDPA | UniProtKB |
| | ASPPRTPAFRADDDDGDEYDDAADAAGDRAPARGREREAPLRGAYPDPTDRL | P08392 |
| | SPRPPAQPPRRRRHGRWRPSASSTSSDSGSSSSSSASSSSSSSDEDEDDDGN | (ICP4_HHV11) |
| | DAADHAREARAVGRGPSSAAPAAPGRTPPPPGPPPLSEAAPKPRAAARTPAA | |
| | SAGRIERRRARAAVAGRDATGRFTAGQPRRVELDADATSGAFYARYRDGYVS | |
| | GEPWPGAGPPPPGRVLYGGLGDSRPGLWGAPEAEEARRRFEASGAPAAVWAP | |
| | ELGDAAQQYALITRLLYTPDAEAMGWLQNPRVVPGDVALDQACFRISGAARN | |
| | SSSFITGSVARAVPHLGYAMAAGRFGWGLAHAAAAVAMSRRYDRAQKGFLLT | |
| | SLRRAYAPLLARENAALTGAAGSPGAGADDEGVAAVAAAAPGERAVPAGYGA | |
| | AGILAALGRLSAAPASPAGGDDPDAARHADADDDAGRRAQAGRVAVECLAAC | |
| | RGILEALAEGFDGDLAAVPGLAGARPASPPRPEGPAGPASPPPPHADAPRLR | |
| | AWLRELRFVRDALVLMRLRGDLRVAGGSEAAVAAVRAVSLVAGALGPALPRD | |
| | PRLPSSAAAAAADLLFDNQSLRPLLAAAASAPDAADALAAAAASAAPREGRK | |
| | RKSPGPARPPGGGGPRPPKTKKSGADAPGSDARAPLPAPAPPSTPPGPEPAP | |
| | AQPAAPRAAAAQARPRPVAVSRRPAEGPDPLGGWRRQPPGPSHTAAPAAAAL | |
| | EAYCSPRAVAELTDHPLFPVPWRPALMFDPRALASIAARCAGPAPAAQAACG | |
| | GGDDDDNPHPHGAAGGRLFGPLRASGPLRRMAAWMRQIPDPEDVRVVVLYSP | |
| | LPGEDLAGGGASGGPPEWSAERGGLSCLLAALANRLCGPDTAAWAGNWTGAP | |
| | DVSALGAQGVLLLSTRDLAFAGAVEFLGLLASAGDRRLIVVNTVRACDWPAD | |
| | GPAVSRQHAYLACELLPAVQCAVRWPAARDLRRTVLASGRVFGPGVFARVEA | |
| | AHARLYPDAPPLRLCRGGNVRYRVRTRFGPDTPVPMSPREYRRAVLPALDGR | |
| | AAASGTTDAMAPGAPDFCEEEAHSHAACARWGLGAPLRPVYVALGREAVRAG | |
| | PARWRGPRRDFCARALLEPDDDAPPLVLRGDDDGPGALPPAPPGIRWASATG | |
| | RSGTVLAAAGAVEVLGAEAGLATPPRREVVDWEGAWDEDDGGAFEGDGVL | |
| ICP4 gene | >JQ673480.1: 146868-150752 Human herpesvirus 1 strain | SEQ ID NO: 4 |
| | KOS, complete genome RS1 | JQ673480.1: |
| | ATGGCGTCGGAGAACAAGCAGCGCCCCGGCTCCCCGGGCCCCACCGACGGGC | 146868-150752 |
| | CGCCGCCCACCCCGAGCCCAGACCGCGACGAGCGGGGGGCCCTCGGGTGGGG | |
| | CGCGGAGACGGAGGAGGGCGGGGACGACCCCGACCACGACCCCGACCACCCC | |
| | CACGACCTCGACGACGCCCGGCGGGACGGGAGGGCCCCCGCGGCGGGCACCG | |
| | ACGCCGGCGAGGACGCCGGGGACGCCGTCTCGTCGCGACAGCTGGCTCTGCT | |
| | GGCCTCCATGGTAGAGGAGGCCGTCCGGACGATCCCGACGCCCGACCCCGCG | |
| | GCCTCGCCGCCCCGGACCCCCGCCTTTCTAGCCGACGACGATGACGGGGACG | |
| | AGTACGACGACGCAGCCGACGCCGCCGGCGACCGGGCCCCGGCCCGGGGCCG | |
| | CGAACGGGAGGCCCCGCTACGCGGCGCGTATCCGGACCCCACGGACCGCCTG | |
| | TCGCCGCGCCCGCCGGCCCAGCCGCCGCGGAGACGTCGTCACGGCCGGCGGC | |
| | GGCCATCGGCGTCATCGACCTCGTCGGACTCCGGGTCCTCGTCCTCGTCGTC | |
| | CGCATCCTCTTCGTCCTCGTCGTCCGACGAGGACGAGGACGACGACGGCAAC | |
| | GACGCGGCCGACCACGCACGCGAGGCGCGGGCCGTCGGGCGGGGTCCGTCGA | |
| | GCGCGGCGCCGGAAGCCCCCGGGCGGACGCCGCCCCCGCCCGGGCCACCCCC | |
| | CCTCTCCGAGGCCGCGCCCAAGCCCCGGGCGGCGGCGAGGACCCCCGCGGCC | |
| | TCCGCGGGCCGCATCGAGCGCCGCCGGGCCCGCGCGGCGGTGGCCGGCCGCG | |
| | ACGCCACGGGCCGCTTCACGGCCGGGCAGCCCCGGCGGGTCGAGCTGGACGC | |
| | CGACGCGGCCTCCGGCGCCTTCTACGCGCGCTATCGCGACGGGTACGTCAGC | |
| | GGGGAGCCGTGGCCCGGCGCCGGGCCCCCGCCCCCGGGGCGGGTGCTGTACG | |
| | GCGGCCTGGGCGACAGCCGCCCGGGCCTCTGGGGGGCGCCCGAGGCGGAGGA | |
| | GGCGCGACGCCGGTTCGAGGCCTCGGGCGCCCCGGCGGCCGTGTGGGCGCCC | |
| | GAGCTGGGCGACGCCGCGCAGCAGTACGCCCTGATCACGCGGCTGCTGTACA | |
| | CCCCGGACGCGGAGGCCATGGGGTGGCTCCAGAACCCGCGCGTGGTCCCCGG | |
| | GGACGTGGCGCTGGACCAGGCCTGCTTCCGGATCTCGGGCGCCGCGCGCAAC | |
| | AGCAGCTCCTTCATCACCGGCAGCGTGGCGCGGGCCGTGCCCCACCTGGGCT | |
| | ACGCCATGGCGGCCGGCCGCTTCGGCTGGGGCCTGGCGCACGCGGCGGCCGC | |
| | CGTGGCCATGAGCCGCCGATACGACCGCGCGCAGAAGGGCTTCCTGCTGACC | |
| | AGCCTGCGCCGCGCCTACGCGCCCCTGTTGGCGCGCGAGAACGCGGCGCTGA | |
| | CGGGGGCCGCGGGGAGCCCCGGCGCCGGCGCAGATGACGAGGGGGTCGCCGC | |
| | CGTCGCCGCCGCCGCACCGGGCGAGCGCGCGGTGCCCGCCGGGTACGGCGCC | |
| | GCGGGGATCCTCGCCGCCCTGGGGCGGCTGTCCGCCGCGCCCGCCTCCCCCG | |
| | TGGGGGGCGACGACCCCGACGCCGCCCGCCACGCCGACGCCGACGCCGGGCG | |
| | CCGCGCCCAGGCCGGCCGCGTGGCCGTCGAGTGCCTGGCCGCCTGCCGCGGG | |
| | ATCCTGGAGGCGCTGGCCGAGGGCTTCGACGGCGACCTGGCGGCCGTCCCGG | |
| | GGCTGGCCGGGGCCCGGCCCGCCAGCCCCCCGCGGCCGGAGGGACCCGCGGG | |
| | CCCCGCTTCCCCGCCGCCGCCGCACGCCGACGCGCCCCGCCTGCGCGCGTGG | |
| | CTGCGCGAGCTGCGGTTCGTGCGCGACGCGCTGGTGCTCATGCGCCTGCGCG | |
| | GGGACCTGCGCGTGGCCGGCGGCAGCGAGGCCGCCGTGGCCGCCGTGCGCGC | |
| | CGTGAGCCTGGTCGCCGGGGCCCTGGGCCCCGCGCTGCCGCGGGACCCGCGC | |
| | CTGCCGAGCTCCGCGGCCGCCGCCGCCGCGGACCTGCTGTTTGAGAACCAGA | |
| | GCCTCCGCCCCCTGCTGGCGGCGGCGGCCAGCGCACCGGACGCCGCCGACGC | |
| | GCTGGCGGCCGCCGCCGCCTCCGCCGCGCCGCGGGAGGGGCGCAAGCGCAAG | |
| | AGTCCCGGCCCGGCCCGGCCGCCCGGAGGCGGCGGCCCGCGACCCCCGAAGA | |
| | CGAAGAAGAGCGGCGCGGACGCCCCCGGCTCGGACGCCCGCGCCCCCCTCCC | |
| | CGCGCCCCCCTCCACGCCCCCGGGGCCCGAGCCCACCCCCGCCCAGCCCGCG | |
| | GCGGCCCGGGGCGCCGCGGCGCAGGCCCGCCCGCGCCCCGTGGCGCTGTCGC | |
| | GCCGGCCCGCCGAGGGCCCCGACCCCCTGGGCGGCTGGCGGCGGCAGCCCCG | |
| | GGGGCCCAGCCACACGGCGGCGCCCGCGGCCGCCGCCCTGGAGGCCTACTGC | |
| | TCCCCGCGCGCCGTGGCCGAGCTCACGGACCACCCGCTGTTCCCCGTCCCCT | |
| | GGCGACCGGCCCTCATGTTTGACCCGCGGGCCCTGGCCTCGATCGCCGCGCG | |
| | GTGCGCCGGGCCCGCCCCCGCCGCCCAGGCCGCGTGCGGCGGCGACGACGAC | |
| | GAGAACCCCCACCCCCACGGGGCCGCCGGGGGCCGCCTCTTTGGCCCCCTGC | |
| | GCGCCTCGGGCCCGCTGCGCCGCATGGCGGCCTGGATGCGCCAGATCCCCGA | |
| | CCCCGAGGACGTGCGCGTGGTGGTGCTGTACTCGCCGCTGCCGGGCGAGGAC | |
| | CTGGCCGGCGGCGGGGCCTCGGGGGGGCCGCCGGAGTGGTCCGCCGAGCGCG | |
| | GCGGGCTGTCCTGCCTGCTGGCGGCCCTGGCCAACCGGCTGTGCGGGCCGGA | |
| | CACGGCCGCCTGGGCGGGCAACTGGACCGGCGCCCCCGACGTGTCGGCGCTG | |
| | GGCGCGCAGGGCGTGCTGCTGCTGTCCACGCGGGACCTGGCCTTCGCCGGGG | |
| | CCGTGGAGTTTCTGGGGCTGCTCGCCAGCGCCGGCGACCGGCGGCTCATCGT | |
| | GGTCAACACCGTGCGCGCCTGCGACTGGCCCGCCGACGGGCCCGCGGTGTCG | |
| | CGGCAGCACGCCTACCTGGCGTGCGACCTGCTGCCCGCCGTGCAGTGCGCCG | |
| | TGCGCTGGCCGGCGGCGCGGGACCTGCGCCGCACGGTGCTGGCCTCGGGCCG | |
| | CGTGTTCGGCCCGGGGGTCTTCGCGCGCGTGGAGGCCGCGCACGCGCGCCTG | |
| | TACCCCGACGCGCCGCCGCTGCGCCTGTGCCGCGGCGGCAACGTGCGCTACC | |
| | GCGTGCGCACGCGCTTCGGCCCGGACACGCCGGTGCCCATGTCCCCGCGCGA | |
| | GTACCGCCGGGCCGTGCTGCCGGCGCTGGACGGCCGGGCGGCGGCCTCGGGG | |
| | ACCACCGACGCCATGGCGCCCGGCGCGCCGGACTTCTGCGAGGAGGAGGCCC | |
| | ACTCGCACCGCGCCTGCGCGCGCTGGGGCCTGGGCGCGCCGCTGCGGCCCGT | |
| | GTACGTGGCGCTGGGGCGCGAGGCGGTGCGCGCCGGCCCGGCCCGGTGGCGC | |
| | GGGCCGCGGAGGGACTTTTGCGCCCGCGCCCTGCTGGAGCCCGACGACGACG | |
| | CCCCCCCGCTGGTGCTGCGCGGCGACGACGACGACGGCCCGGGGGCCCTGCC | |
| | GCCGGCGCCGCCCGGGATTCGCTGGGCCTCGGCCACGGGCCGCAGCGGCACC | |
| | GTGCTGGCGGCGGCGGGGGCCGTGGAGGTGCTGGGGGCGGAGGCGGGCTTGG | |
| | CCACGCCCCCGCGACGGGACGTTGTGGACTGGGAAGGCGCCTGGGACGAAGA | |
| | CGACGGCGGCGCGTTCGAGGGGGACGGGGTGCTGTAA | |
| ICP22 | MADISPGAFAPCVKARRPALRSPPLGTRKRKRPSRPLSSESEVESDTALESE | SEQ ID NO: 5 |
| polypeptide | VESETASD | UniProtKB- |
| | STESGDQDEAPRIGGRRAPRRLGGRFFLDMSAESTTGTETDASVSDDPDDTS | P04485 |
| | DWSYDDIP | (ICP22_HHV11) |
| | PRPKRARVNLRLTSSPDRRDGVIFPKMGRVRSTRETQPRAPTPSAPSPNAML | |
| | RRSVRQAQ | |
| | RRSSARWTPDLGYMRQCINQLFRVLRVARDPHGSANRLRHLIRDCYLMGYCR | |
| | ARLAPRTW | |
| | CRLLQVSGGTWGMHLRNTIREVEARFDATAEPVCKLPCLETRRYGPECDLSN | |
| | LEIHLSAT | |
| | SDDEISDATDLEAAGSDHTLASQSDTEDAPSPVTLETPEPRGSLAVRLEDEF | |
| | GEFDWTPQ | |
| | EGSQPWLSAVVADTSSVERPGPSDSGAGRAAEDRKCLDGCRKMRFSTACPYP | |
| | CSDTFLRP | |
| ICP22 gene | NCBI: JQ673480.1 (130165-132995) | SEQ ID NO: 6 |
| sequence | | |
| ICP27 | MATDIDMLIDLGLDLSDSDLDEDPPEPAESRRDDLESDSSGECSSSDEDMED | SEQ ID NO: 7 |
| polypeptide | PHGEDGPEPILDAARPAVRPSRPEDPGVPSTQTPRPTERQGPNDPQPAPHSV | UniProtKB- |
| | WSRLGARRPSCSPEQHGGKVARLQPPPTKAQPARGGRRGRRRGRGRGGPGAA | P10238 |
| | DGLSDPRRRAPRTNRNPGGPRPGAGWTDGPGAPHGEAWRGSEQPDPPGGQRT | (ICP27_HHV11) |
| | RGVRQAPPPLMTLAIAPPPADPRAPAPERKAPAADTIDATTRLVLRSISERA | |
| | AVDRISESFGRSAQVMHDPFGGQPFPAANSPWAPVLAGQGGPFDAETRRVSW | |
| | ETLVAHGPSLYRTFAGNPRAASTAKAMRDCVLRQENFIEALASADETLAWCK | |
| | MCIHHNLPLRPQDPIIGTTAAVLDNLATRLRPFLQCYLKARGLCGLDELCSR | |
| | RRLADIKDIASFVFVILARLANRVERGVAEIDYATLGVGVGEKMHFYLPGAC | |
| | MAGLIEILDTHRQECSSRVCELTASHIVAPPYVHGKYFYCNSLF | |
| ICP27 gene | >JQ673480.1: 113656-115194 Human herpesvirus 1 strain | SEQ ID NO: 8 |
| | KOS, complete genome UL54 | JQ673480.1: |
| | ATGGCGACTGACATTGATATGCTAATTGACCTCGGCCTGGACCTCTCCGACA | 113656-115194 |
| | GCGATCTGGACGAGGACCCCCCCGAGCCGGCGGAGAGCCGCCGCGACGACCT | |
| | GGAATCGGACAGCAACGGGGAGTGTTCCTCGTCGGACGAGGACATGGAAGAC | |
| | CCCCACGGAGAGGACGGACCGGAGCCGATACTCGACGCCGCTCGCCCGGCGG | |
| | TCCGCCCGTCTCGTCCAGAAGACCCCGGCGTACCCAGCACCCAGACGCCTCG | |
| | TCCGACGGAGCGGCAGGGCCCCAACGATCCTCAACCAGCGCCCCACAGTGTG | |
| | TGGTCGCGCCTCGGGGCCCGGCGACCGTCTTGCTCCCCCGAGCGGCACGGGG | |
| | GCAAGGTGGCCCGCCTCCAACCCCCACCGACCAAAGCCCAGCCTGCCCGCGG | |
| | CGGACGCCGTGGGCGTCGCAGGGGTCGGGGTCGCGGTGGTCCCGGGGCCGCC | |
| | GATGGTTTGTCGGACCCCCGCCGGCGTGCCCCCAGAACCAATCGCAACCCGG | |
| | GGGGACCCCGCCCCGGGGCGGGGTGGACGGACGGCCCCGGCGCCCCCCATGG | |
| | CGAGGCGTGGCGCGGAAGTGAGCAGCCCGACCCACCCGGAGGCCCGCGGACA | |
| | CGGAGCGTGCGCCAAGCACCCCCCCCGCTAATGACGCTGGCGATTGCCCCCC | |
| | CGCCCGCGGACCCCCGCGCCCCGGCCCCGGAGCGAAAGGCGCCCGCCGCCGA | |
| | CACCATCGACGCCACCACGCGGTTGGTCCTGCGCTCCATCTCCGAGCGCGCG | |
| | GCGGTCGACCGCATCAGCGAGAGCTTCGGCCGCAGCGCACAGGTCATGCACG | |
| | ACCCCTTTGGGGGGCAGCCGTTTCCCGCCGCGAATAGCCCCTGGGCCCCGGT | |
| | GCTGGCGGGCCAAGGAGGGCCCTTTGACGCCGAGACCAGACGGGTCTCCTGG | |
| | GAAACCTTGGTCGCCCACGGCCCGAGCCTCTATCGCACTTTTGCCGGCAATC | |
| | CTCGGGCCGCATCGACCGCCAAGGCCATGCGCGACTGCGTGCTGCGCCAAGA | |
| | AAATTTCATCGAGGCGCTGGCCTCCGCCGACGAGACGCTGGCGTGGTGCAAG | |
| | ATGTGCATCCACCACAACCTGCCGCTGCGCCCCCAGGACCCCATTATCGGGA | |
| | CGGCCGCGGCGGTGCTGGATAACCTCGCCACGCGCCTGCGGCCCTTTCTCCA | |
| | GTGCTACCTGAAGGCGCGAGGCCTGTGCGGCCTGGACGAACTGTGTTCGCGG | |
| | CGGCGTCTGGCGGACATTAAGGACATTGCATCCTTCGTGTTTGTCATTCTGG | |
| | CCAGGCTCGCCAACCGCGTCGAGCGTGGCGTCGCGGAGATCGACTACGCGAC | |
| | CCTTGGTGTCGGGGTCGGAGAGAAGATGCATTTCTACCTCCCCGGGGCCTGC | |
| | ATGGCGGGCCTGATCGAAATCCTAGACACGCACCGCCAGGAGTGTTCGAGTC | |
| | GTGTCTGCGAGTTGACGGCCAGTCACATCGTCGCCCCCCCGTACGTGCACGG | |
| | CAAATATTTTTATTGCAACTCCCTGTTTTAG | |
| ICP47 | MSWALEMADTFLDTMRVGPRTYADVRDEINKRGREDREAARTAVHDPERPLL | SEQ ID NO: 9 |
| polypeptide | RSPGLLPEIAPNASLGVAHRRTGGTVTDSPRNPVTR | UniProtKB- |
| | | P03170 |
| | | (ICP47_HHV11) |
| ICP47 gene | >JQ673480.1: 145118-145384 Human herpesvirus 1 strain | SEQ ID NO: 10 |
| | KOS, complete genome US12 | JQ673480.1: |
| | TCAACGGGTTACCGGATTACGGGGACTGTCGGTCACGGTCCCGCCGGTTCTT | 145118-145384 |
| | CGATGTGCCACACCCAAGGATGCGTTGGGGGCGATTTCGGGCAGCAGCCCGG | |
| | GAGAGCGCAGCAGGGGACGCTCCGGGTCGTGCACGGCGGTTCTGGCCGCCTC | |
| | CCGGTCCTCACGCCCCCTTTTATTGATCTCATCGCGTACGTCGGCGTACGTC | |
| | CTGGGCCCAACCCGCATGTTGTCCAGGAAGGTGTCCGCCATTTCCAGGGCCC | |
| | ACGACAT | |

In some embodiments of any of the aspects, the genome comprises a nucleotide sequence of SEQ ID NO: 2 and where there is at least one alteration in SEQ ID NO: 2.

In some embodiments of any of the aspects, the genome comprises a nucleotide sequence of SEQ ID NO: 4 and where there is at least one alteration in SEQ ID NO: 4.

In some embodiments of any of the aspects, the genome comprises a nucleotide sequence of SEQ ID NO: 6 and where there is at least one alteration in SEQ ID NO: 6.

In some embodiments of any of the aspects, the genome comprises a nucleotide sequence of SEQ ID NO: 8 and where there is at least one alteration in SEQ ID NO: 8.

In some embodiments of any of the aspects, the genome comprises a nucleotide sequence of SEQ ID NO: 10 and where there is at least one alteration in SEQ ID NO: 10.

In some embodiments of any of the aspects, the genome comprises a 271 nucleotide deletion in ICP22 promoter from 131396-131666 (NCBI Ref sequence: JQ673480.1) (SEQ ID NO: 11).

In some embodiments of any of the aspects, the genome comprises a 271 nucleotide deletion in ICP47 promoter from 146188-146458 (NCBI Reference sequence: JQ673480.1) (SEQ ID NO: 12).

In some embodiments of any of the aspects, the genome further comprises at least one alteration in a U_{L}41 loci.

In some embodiments of any of the aspects, the genome further comprises at least one alteration in a S component repeated sequence. In some embodiments of any of the aspects, the genome further comprises at least one alteration in a S component repeated sequence in a promoter between a gene encoding ICP4 and a gene encoding ICP22 or in a promoter between a gene encoding ICP4 and a gene encoding ICP47. The S component repeat sequence between ICP4 and ICP22 is as follows:

(SEQ ID NO: 13)
CGCCGATGCGGGGCGATCCTCCGGGGATACGGCTGCGACGGCGGACGTA
GCACGGTAGGTCACCTACGGACTCTCGATGGGGGGAGGGGGCGAGACCC
ACGGACCCCGACGACCCCCGCCGTCGACGCGGAACTAGCGCGGACCGGT
CGATGCTTGGGTGGGGAAAAAGGACAGGGACGGCCGATCCCCCTCCCGC
GCTTCGTCCGCGTATCGGCGTCCCGGCGCGGCGAGCGTCTGACGGTCTG
TCTCTGGCGGTCCCGCGTCGGGTCGTGGATCCGTGTCGGCAGCCGCGCT
CCGTGTGGACGATCGGGGCGTCCTCGGGCTCATATAGTCCCAGGGGCCG
GCGGGAAGGAGGAGCAGCGGAGGCCGCCGGCCCCCCGCCCCCCGGCGGG
CCCACCCCGAACGGAATTCCATTATGCACGACCCCGCCCCGACGCCGGC
ACGCCGGGGGCCCGTGGCCGCGGCCCGTTGGTCGAACCCCCGGCCCCGC
CCATCCGCGCCATCTGCCATGGACGGGGCGCGAGGGCGGGTGGGTCCGC
GCCCCGCCCCGCATGGCATCTCATTACCGCCCGATCCGGCGGTTTCCGC
TTCCGTTCCGCATGCTAACGAGGAACGGGCAGGGGGCGGGGCCCGGGCC
CCGACTTCCCGGTTCGGCGGTAATGAGATACGAGCCCCGCGCGCCCGTT
GGCCGTCCCCGGGCCCCCGGTCCCGCCCGCCGGACGCCGGGACCAACGG
GACGGCGGGCGGCCCTTGGGCCGCCCGCCTTGCCGCCCCCCCATTGGCC
GGCGGGCGGGACCGCCCCAAGGGGGCGGGGCCGCCGGGTAAAAGAAGTG
AGAACGCGAAGCGTTCGCACTTCGTCCCAATATATATATATTATTAGGG
CGAAGTGCGAGCACTGGCGCCGTGCCCGACTCCGCGCCGGCCCCGGGGG
CGGACCCGGGCGGCGGGGGGCGGGTCTCTCCGGCGCACATAAAGGCCCG
GCGCGACCGACGCCCGCAGACGGCGCCAGCCACGAACGACGGGAGCGGC
TGCGGAGCACGCGGACCGGGAGCGGGAGTCGCAGAGGGCCGTCGGAGCG
GACGGCGTCGGCATCGCGACGCCCCGGCTCGGGATCGGGATCGCATCGG
AAAGGGACACGCGGACGCGGGGGGGAAAGACCCGCCCACCCCACCCACG
AAACACAGGGGACGCACCCCGGGGGCCTCCGACGACAGAAACCCACCGG
TCCGCCTTTTTTGCACGGGTAAGCACCTTGGGTGGGCAGAGGAGGGGGG
ACGCGGGGGCGGAGGAGGGGGGACGCGGGGGCGGAGGAGGGGGGACGCG
GGGGCGGAGGAGGGGGGACGCGGGGGCGGAGGAGGGGGGACGCGGGGGC
GGAGGAGGGGGCTCACCCGCGTTCGTGCCTTCCCGCAGGAGGAACGCCC
TCGTCGAGGCGACCGGCGGCGACCGTTGCGTGGACCGCTTCCTGCTCGT
CGGG.

In some embodiments of any of the aspects, the genome comprises a nucleotide sequence of SEQ ID NO: 13 and where there is at least one alteration in SEQ ID NO: 13.

In some embodiments of any of the aspects, the genome lacks at least one Oct-1 site present in wild-type HSV-1 genome. In some embodiments of any of the aspects, the genome lacks at least one Oct-1 site present in a promoter of a gene encoding ICP22 or a gene encoding ICP47.

In some embodiments of any of the aspects, the genome lacks at least one (e.g., one, two or three) SP1 binding site present in wild-type HSV-1 genome. In some embodiments of any of the aspects, the genome lacks at least one (e.g., one, two or three) SP1 binding site present in a promoter of a gene encoding ICP22 or a gene encoding ICP47.

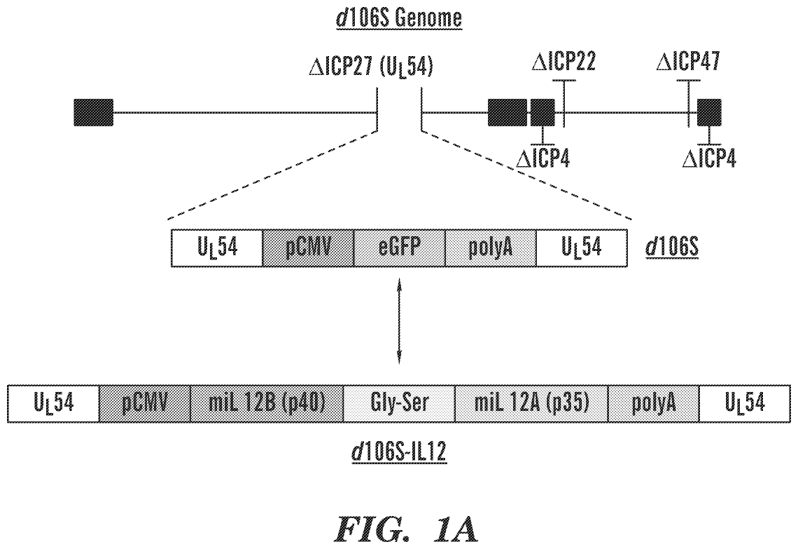

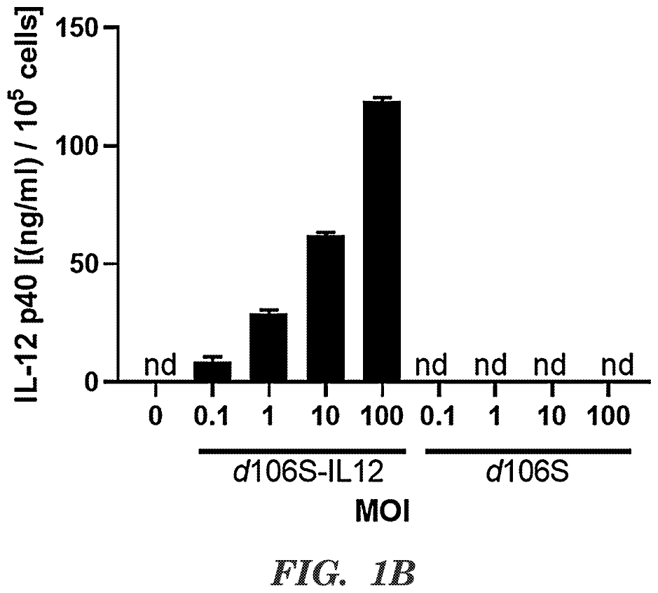

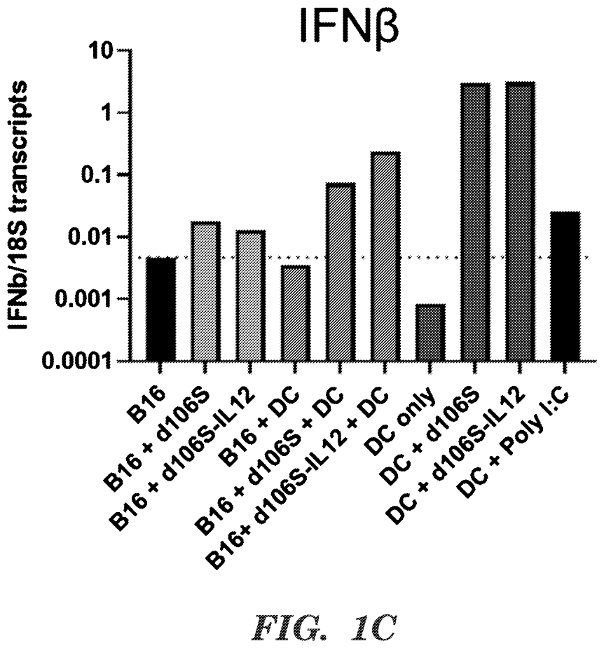

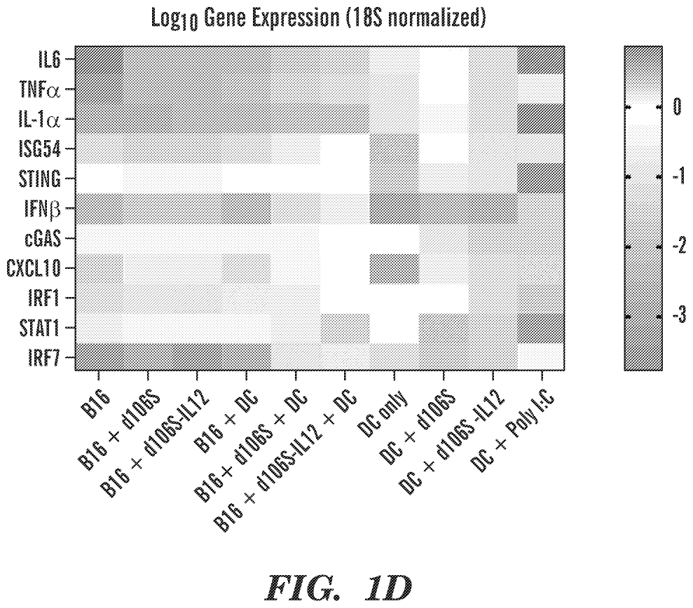

In some embodiments of any of the aspects, the HSV is d106S. A schematic representation of the d106S is demonstrated in FIG. 1A.

Generally, the genome of the recombinant virus comprises a therapeutic gene coding sequence. As used herein, the term “therapeutic gene” refers to a gene that, when expressed, confers a beneficial effect on the cell or tissue in which it is present, or on a subject, e.g., a mammal in which the gene is expressed. Examples of beneficial effects include amelioration of a sign or symptom of a condition or disease, prevention or inhibition of a condition or disease, or conferral of a desired characteristic. It is noted that the genome of the recombinant virus as described herein can comprise two, three, four, five, six, seven, eight, nine, ten or more therapeutic gene coding sequences. The therapeutic gene coding sequences can be the same or different or some same and some different.

As described herein, the therapeutic gene coding sequence can encode a non-fusion or a fusion protein. Accordingly, in some embodiments of the various aspects, the therapeutic gene coding sequence encodes a heterodimeric protein as a fusion protein. For example, the therapeutic gene coding sequence encodes a first domain of the fusion protein and a second domain of the fusion protein, where the first and second domain are different domains of the heterodimeric protein.

In some embodiments of the various aspects, the therapeutic gene coding sequence encodes a cytokine. Exemplary cytokines include, but are not limited to interleukin 12 (IL-12), IL-2, IL-3, IL-4, IL-5, IL-6, IL-7, IL-9, IL-10, IL-12, IL-15, IL-17, IL-18, and interferon (IFN)-γ. Accordingly, in some embodiments of the various aspects, the therapeutic gene coding sequence encodes IL-12, IL-2, IL-3, IL-4, IL-5, IL-6, IL-7, IL-9, IL-10, IL-12, IL-15, IL-17, IL-18, and interferon (IFN)-γ or any combinations thereof

In some embodiments of any of the aspects, the therapeutic gene coding sequence encodes a heterodimeric cytokine. For example, the therapeutic gene coding sequences encodes IL-12. As IL-12 is a heterodimer, the therapeutic gene coding sequences can encode a fusion protein comprising a p40 subunit of IL-12 and a p34 subunit of IL-12 as separate domains. The p40 and p35 domains can be linked via a linker.

IL-12

IL-12 is a heterodimeric cytokine composed of p35 (also referred to herein as IL12A) and p40 (also referred to herein as IL12B) subunits that is capable of eliciting a variety of immune responses in the body. Native IL-12 is produced by activated antigen-presenting cells (e.g, dendritic cells, macrophages). The IL-12 polypeptide promotes the development of Thl responses by the immune system and activates the induction of IFNγ production by T cells and Natural Killer (NK) cells. Additional immunological functions produced by IL-12 are described in detail by Vignali, D., et al. IL-12 family cytokines: immunological playmakers. Nat Immunol 13, 722-728 (2012); Ling P, et al. Human IL-12 p40 homodimer binds to the IL-12 receptor but does not mediate biologic activity. J Immunol. 1995;154(1):116-127; and Brunda M J, et al. Antitumor and Antimetastatic Activity of Interleukin 12 against Murine Tumors. J Exp Med. 1993, which are incorporated herein by reference in its entirety. Full-length and partial sequences of IL-12, variants and isoforms thereof are known in the art. For example, the human and mouse gene, mRNA, and amino acid sequences are demonstrated in the following table.

TABLE 2
Human and Mouse IL-12 Sequences

| SEQ ID NO: | Sequence type | Reference sequence |
|---|---|---|
| SEQ ID NO: 14 | IL12A Gene (p35) | NCBI Ref Sequence: |
| | [Homo sapiens] | >NC_000003.12: 159988835-159996019 |
| | | Homo sapiens chromosome 3, GRCh38.p13 |
| | | Primary Assembly |
| SEQ ID NO: 15 | Homo sapiens | NCBI Reference Sequence: NM_000882.4 |
| | interleukin 12A | atttcgctttcattttgggccgagctggaggcg |
| | (IL12A), transcript | gcggggccgtcccggaacggctgcggccgggca |
| | variant 1, mRNA | ccccgggagttaatccgaaagcgccgcaagccc |
| | | cgcgggccggccgcaccgcacgtgtcaccgaga |
| | | agctgatgtagagagagacacagaaggagacag |
| | | aaagcaagagaccagagtcccgggaaagtcctg |
| | | ccgcgcctcgggacaattataaaaatgtggccc |
| | | cctgggtcagcctcccagccaccgccctcacct |
| | | gccgcggccacaggtctgcatccagcggctcgc |
| | | cctgtgtccctgcagtgccggctcagcatgtgt |
| | | ccagcgcgcagcctcctccttgtggctaccctg |
| | | gtcctcctggaccacctcagtttggccagaaac |
| | | ctccccgtggccactccagacccaggaatgttc |
| | | ccatgccttcaccactcccaaaacctgctgagg |
| | | gccgtcagcaacatgctccagaaggccagacaa |
| | | actctagaattttacccttgcacttctgaagag |
| | | attgatcatgaagatatcacaaaagataaaacc |
| | | agcacagtggaggcctgtttaccattggaatta |
| | | accaagaatgagagttgcctaaattccagagag |
| | | acctctttcataactaatgggagttgcctggcc |
| | | tccagaaagacctcttttatgatggccctgtgc |
| | | cttagtagtatttatgaagacttgaagatgtac |
| | | caggtggagttcaagaccatgaatgcaaagctt |
| | | ctgatggatcctaagaggcagatctttctagat |
| | | caaaacatgctggcagttattgatgagctgatg |
| | | caggccctgaatttcaacagtgagactgtgcca |
| | | caaaaatcctcccttgaagaaccggatttttat |
| | | aaaactaaaatcaagctctgcatacttcttcat |
| | | gctttcagaattcgggcagtgactattgataga |
| | | gtgatgagctatctgaatgcttcctaaaaagcg |
| | | aggtccctccaaaccgttgtcatttttataaaa |
| | | ctttgaaatgaggaaactttgataggatgtgga |
| | | ttaagaactagggagggggaaagaaggatggga |
| | | ctattacatccacatgatacctctgatcaagta |
| | | tttttgacatttactgtggataaattgttttta |
| | | agttttcatgaatgaattgctaagaagggaaaa |
| | | tatccatcctgaaggtgtttttcattcacttta |
| | | atagaagggcaaatatttataagctatttctgt |
| | | accaaagtgtttgtggaaacaaacatgtaagca |
| | | taacttattttaaaatatttatttatataactt |
| | | ggtaatcatgaaagcatctgagctaacttatat |
| | | ttatttatgttatatttattaaattatttatca |
| | | agtgtatttgaaaaatatttttaagtgttctaa |
| | | aaataaaagtattgaattaaagtga |
| SEQ ID NO: 16 | interleukin-12 | NCBI Reference Sequence: NP_000873.2 |
| | subunit alpha isoform | mwppgsasqpppspaaatglhpaarpvslqcrlsmc |
| | 1 precursor | parslllvatlvlldhlslarnlpvatpdpgmfpcl |
| | [Homo sapiens] amino | hhsqnllravsnmlqkarqtlefypctseeidhedi |
| | acid sequence | tkdktstveaclpleltknesclnsretsfitngsc |
| | | lasrktsfmmalclssiyedlkmyqvefktmnakll |
| | | mdpkrqifldqnmlavidelmqalnfnsetvpqkss |
| | | leepdfyktkiklcillhafriravtidrvmsylna |
| | | s |
| SEQ ID NO: 17 | IL12B Gene | >NC_000005.10: c159330487-159314780 |
| | (p40) [Homo sapiens] | Homo sapiens chromosome 5, GRCh39.p13 |
| | | Primary Assembly |
| SEQ ID NO: 18 | Homo sapiens | NCBI Reference Sequence: NM_002187.3 |
| | interleukin 12B | agaagaaacaacatctgtttcagggccattggactc |
| | (IL12B), mRNA | tccgtcctgcccagagcaagatgtgtcaccagcagt |
| | | tggtcatctcttggttttccctggtttttctggcat |
| | | ctcccctegtggccatatgggaactgaagaaagatg |
| | | tttatgtcgtagaattggattggtatccggatgccc |
| | | ctggagaaatggtggtcctcacctgtgacacccctg |
| | | aagaagatggtatcacctggaccttggaccagagca |
| | | gtgaggtcttaggctctggcaaaaccctgaccatcc |
| | | aagtcaaagagtttggagatgctggccagtacacct |
| | | gtcacaaaggaggcgaggttctaagccattcgctcc |
| | | tgctgcttcacaaaaaggaagatggaatttggtcca |
| | | ctgatattttaaaggaccagaaagaacccaaaaata |
| | | agacctttctaagatgcgaggccaagaattattctg |
| | | gacgtttcacctgctggtggctgacgacaatcagta |
| | | ctgatttgacattcagtgtcaaaagcagcagaggct |
| | | cttctgacccccaaggggtgacgtgcggagctgcta |
| | | cactctctgcagagagagtcagaggggacaacaagg |
| | | agtatgagtactcagtggagtgccaggaggacagtg |
| | | cctgcccagctgctgaggagagtctgcccattgagg |
| | | tcatggtggatgccgttcacaagctcaagtatgaaa |
| | | actacaccagcagcttcttcatcagggacatcatca |
| | | aacctgacccacccaagaacttgcagctgaagccat |
| | | taaagaattctcggcaggtggaggtcagctgggagt |
| | | accctgacacctggagtactccacattcctacttct |
| | | ccctgacattctgcgttcaggtccagggcaagagca |
| | | agagagaaaagaaagatagagtcttcacggacaaga |
| | | cctcagccacggtcatctgccgcaaaaatgccagca |
| | | ttagcgtgcgggcccaggaccgctactatagctcat |
| | | cttggagcgaatgggcatctgtgccctgcagttagg |
| | | ttctgatccaggatgaaaatttggaggaaaagtgga |
| | | agatattaagcaaaatgtttaaagacacaacggaat |
| | | agacccaaaaagataatttctatctgatttgcttta |
| | | aaacgtttttttaggatcacaatgatatctttgctg |
| | | tatttgtatagttagatgctaaatgctcattgaaac |
| | | aatcagctaatttatgtatagattttccagctctca |
| | | agttgccatgggccttcatgctatttaaatatttaa |
| | | gtaatttatgtatttattagtatattactgttattt |
| | | aacgtttgtctgccaggatgtatggaatgtttcata |
| | | ctcttatgacctgatccatcaggatcagtccctatt |
| | | atgcaaaatgtgaatttaattttatttgtactgaca |
| | | acttttcaagcaaggctgcaagtacatcagttttat |
| | | gacaatcaggaagaatgcagtgttctgataccagtg |
| | | ccatcatacacttgtgatggatgggaacgcaagaga |
| | | tacttacatggaaacctgacaatgcaaacctgttga |
| | | gaagatccaggagaacaagatgctagttcccatgtc |
| | | tgtgaagacttcctggagatggtgttgataaagcaa |
| | | tttagggccacttacacttctaagcaagtttaatct |
| | | ttggatgcctgaattttaaaagggctagaaaaaaat |
| | | gattgaccagcctgggaaacataacaagaccccgtc |
| | | tctacaaaaaaaatttaaaattagccaggcgtggtg |
| | | gctcatgcttgtggtcccagctgttcaggaggatga |
| | | ggcaggaggatctcttgagcccaggaggtcaaggct |
| | | atggtgagccgtgattgtgccactgcataccagcct |
| | | aggtgacagaatgagaccctgtctcaaaaaaaaaaa |
| | | tgattgaaattaaaattcagctttagcttccatggc |
| | | agtcctcacccccacctctctaaaagacacaggagg |
| | | atgacacagaaacaccgtaagtgtctggaaggcaaa |
| | | aagatcttaagattcaagagagaggacaagtagtta |
| | | tggctaaggacatgaaattgtcagaatggcaggtgg |
| | | cttcttaacagccctgtgagaagcagacagatgcaa |
| | | agaaaatctggaatccctttctcattagcatgaatg |
| | | aacctgatacacaattatgaccagaaaatatggctc |
| | | catgaaggtgctacttttaagtaatgtatgtgcgct |
| | | ctgtaaagtgattacatttgtttcctgtttgtttat |
| | | ttatttatttatttttgcattctgaggctgaactaa |
| | | taaaaactcttctttgtaatcata |
| SEQ ID NO: 19 | interleukin-12 | NCBI Reference Sequence: NP_002178.2 |
| | subunit beta | mchqqlviswfslvflasplvaiwelkkdvyvveld |
| | precursor amino acid | wypdapgemvvltcdtpeedgitwtldqssevlgsg |
| | sequence | ktltiqvkefgdagqytchkggevlshs1111hkke |
| | [Homo sapiens] | dgiwstdilkdqkepknktflrceaknysgrftcww |
| | | Ittistdltfsvkssrgssdpqgvtcgaatlsaerv |
| | | rgdnkeyeysvecqedsacpaaeeslpievmvdavh |
| | | klkyenytssffirdiikpdppknlqlkplknsrqv |
| | | evsweypdtwstphsyfsltfcvqvqgkskrekkdr |
| | | vftdktsatvicrknasisvraqdryyssswsewas |
| | | vpcs |
| SEQ ID NO: 20 | Mus musculus IL12A | NCBI NM_001159424.2 |
| | coding sequence | ATGGTCAGCGTTCCAACAGCCTCACCCTCGGCATCC |
| | (p35) | AGCAGCTCCTCTCAGTGCCGGTCCAGCATGTGTCAA |
| | | TCACGCTACCTCCTCTTTTTGGCCACCCTTGCCCTC |
| | | CTAAACCACCTCAGTTTGGCCAGGGTCATTCCAGTC |
| | | TCTGGACCTGCCAGGTGTCTTAGCCAGTCCCGAAAC |
| | | CTGCTGAAGACCACAGATGACATGGTGAAGACGGCC |
| | | AGAGAAAAACTGAAACATTATTCCTGCACTGCTGAA |
| | | GACATCGATCATGAAGACATCACACGGGACCAAACC |
| | | AGCACATTGAAGACCTGTTTACCACTGGAACTACAC |
| | | AAGAACGAGAGTTGCCTGGCTACTAGAGAGACTTCT |
| | | TCCACAACAAGAGGGAGCTGCCTGCCCCCACAGAAG |
| | | ACGTCTTTGATGATGACCCTGTGCCTTGGTAGCATC |
| | | TATGAGGACTTGAAGATGTACCAGACAGAGTTCCAG |
| | | GCCATCAACGCAGCACTTCAGAATCACAACCATCAG |
| | | CAGATCATTCTAGACAAGGGCATGCTGGTGGCCATC |
| | | GATGAGCTGATGCAGTCTCTGAATCATAATGGCGAG |
| | | ACTCTGCGCCAGAAACCTCCTGTGGGAGAAGCAGAC |
| | | CCTTACAGAGTGAAAATGAAGCTCTGCATCCTGCTT |
| | | CACGCCTTCAGCACCCGCGTCGTGACCATCAACAGG |
| | | GTGATGGGCTATCTGAGCTCCGCC |
| SEQ ID NO: 21 | Mus musculus IL12A | Uniprot P43431; NP_001152896.1 |
| | amino acid sequence | MVSVPTASPSASSSSSQCRSSMCQSRYLLFLATLAL |
| | (p35) | LNHLSLARVIPVSGPARCLSQSRNLLKTTDDMVKTA |
| | | REKLKHYSCTAEDIDHEDITRDQTSTLKTCLPLELH |
| | | KNESCLATRETSSTTRGSCLPPQKTSLMMTLCLGSI |
| | | YEDLKMYQTEFQAINAALQNHNHQQIILDKGMLVAI |
| | | DELMQSLNHNGETLRQKPPVGEADPYRVKMKLCILL |
| | | HAFSTRVVTINRVMGYLSSA |
| SEQ ID NO: 22 | Mus musculus IL12B | NCBI NM 001303244.1 |
| | coding sequence | ATGTGTCCTCAGAAGCTAACCATCTCCTGGTTTGCC |
| | (p40) | ATCGTTTTGCTGGTGTCTCCACTCATGGCCATGTGG |
| | | GAGCTGGAGAAAGACGTTTATGTTGTAGAGGTGGAC |
| | | TGGACTCCCGATGCCCCTGGAGAAACAGTGAACCTC |
| | | ACCTGTGACACGCCTGAAGAAGATGACATCACCTGG |
| | | ACCTCAGACCAGAGACATGGAGTCATAGGCTCTGGA |
| | | AAGACCCTGACCATCACTGTCAAAGAGTTTCTAGAT |
| | | GCTGGCCAGTACACCTGCCACAAAGGAGGCGAGACT |
| | | CTGAGCCACTCACATCTGCTGCTCCACAAGAAGGAA |
| | | AATGGAATTTGGTCCACTGAAATTTTAAAAAATTTC |
| | | AAAAACAAGACTTTCCTGAAGTGTGAAGCACCAAAT |
| | | TACTCCGGACGGTTCACGTGCTCATGGCTGGTGCAA |
| | | AGAAACATGGACTTGAAGTTCAACATCAAGAGCAGT |
| | | AGCAGTTCCCCTGACTCTCGGGCAGTGACATGTGGA |
| | | ATGGCGTCTCTGTCTGCAGAGAAGGTCACACTGGAC |
| | | CAAAGGGACTATGAGAAGTATTCAGTGTCCTGCCAG |
| | | GAGGATGTCACCTGCCCAACTGCCGAGGAGACCCTG |
| | | CCCATTGAACTGGCGTTGGAAGCACGGCAGCAGAAT |
| | | AAATATGAGAACTACAGCACCAGCTTCTTCATCAGG |
| | | GACATCATCAAACCAGACCCGCCCAAGAACTTGCAG |
| | | ATGAAGCCTTTGAAGAACTCACAGGTGGAGGTCAGC |
| | | TGGGAGTACCCTGACTCCTGGAGCACTCCCCATTCC |
| | | TACTTCTCCCTCAAGTTCTTTGTTCGAATCCAGCGC |
| | | AAGAAAGAAAAGATGAAGGAGACAGAGGAGGGGTGT |
| | | AACCAGAAAGGTGCGTTCCTCGTAGAGAAGACATCT |
| | | ACCGAAGTCCAATGCAAAGGCGGGAATGTCTGCGTG |
| | | CAAGCTCAGGATCGCTATTACAATTCCTCATGCAGC |
| | | AAGTGGGCATGTGTTCCCTGCAGGGTCCGATCC |
| SEQ ID NO: 23 | Mus musculus IL 12B | Uniprot P43432; NP_001290173.1 |
| | amino acid sequence | MCPQKLTISWFAIVLLVSPLMAMWELEKDVYVVEVD |
| | (p40) | WTPDAPGETVNLTCDTPEEDDITWTSDQRHGVIGSG |
| | | KTLTITVKEFLDAGQYTCHKGGETLSHSHLLLHKKE |
| | | NGIWSTEILKNFKNKTFLKCEAPNYSGRFTCSWLVQ |
| | | RNMDLKFNIKSSSSSPDSRAVTCGMASLSAEKVTLD |
| | | QRDYEKYSVSCQEDVTCPTAEETLPIELALEARQQN |
| | | KYENYSTSFFIRDIIKPDPPKNLQMKPLKNSQVEVS |
| | | WEYPDSWSTPHSYFSLKFFVRIQRKKEKMKETEEGC |
| | | NQKGAFLVEKTSTEVQCKGGNVCVQAQDRYYNSSCS |
| | | KWACVPCRVRS |

In some embodiments of any of the aspects, the recombinant virus provided herein comprises within its genome a therapeutic gene coding sequence encoding an IL-12 fusion protein. In some embodiments of any of the aspects, the fusion protein comprises a first domain and a second domain. For example, one of the first and second domain encodes a p40 subunit of IL-12 and the other domain encodes a p35 subunit of IL-12.

In some embodiments of any of the aspects, the therapeutic gene coding sequence comprises nucleotide sequence encoding a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence selected from the group consisting of SEQ ID NO: 16, SEQ ID NO: 19, SEQ ID NO: 21, SEQ ID NO: 23, and any combinations thereof. For example, the therapeutic gene coding sequence comprises a nucleotide sequence encoding a polypeptide comprising an amino acid sequence having at least 90%, 91%, 92%, 93%, 94%, 95%, 96%, 97%, 98% or 99% identity to an amino acid sequence selected from the group consisting of SEQ ID NO: 16, SEQ ID NO: 19, SEQ ID NO: 21, SEQ ID NO: 23, and any combinations thereof.

In some embodiments of any of the aspects, the therapeutic gene coding sequence comprises nucleotide sequence encoding a polypeptide comprising a first amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 16 or SEQ ID NO: 21, and further comprises SEQ ID NO: 17, SEQ ID NO: 19, SEQ ID NO: 21, and a second amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 19 or SEQ ID NO: 21. For example, the therapeutic gene coding sequence comprises nucleotide sequence encoding a polypeptide comprising a first amino acid sequence having at least 90%, 91%, 92%, 93%, 94%, 95%, 96%, 97%, 98% or 99% identity to SEQ ID NO: 16 or SEQ ID NO: 21, and further comprises SEQ ID NO: 19, SEQ ID NO: 21, SEQ ID NO: 23, and a second amino acid sequence having at least 90%, 91%, 92%, 93%, 94%, 95%, 96%, 97%, 98% or 99% identity to SEQ ID NO: 19 or SEQ ID NO: 21.

In some embodiments of any of the aspects, the therapeutic gene coding sequence comprises a nucleotide sequence selected from the group consisting of SEQ ID NO: 14, SEQ ID 15, SEQ ID NO: 17, SEQ ID NO: 18, SEQ ID NO: 20, SEQ ID NO: 22, and any combinations thereof.

In some embodiments of any of the aspects, the therapeutic gene coding sequence comprises a nucleotide sequence selected from the group consisting of SEQ ID NO: 14, SEQ ID 15 and SEQ ID NO: 20.

In some embodiments of any of the aspects, the therapeutic gene coding sequence comprises a nucleotide sequence selected from the group consisting of SEQ ID NO: 17, SEQ ID NO: 18, SEQ ID NO: 22, and any combinations thereof.

In some embodiments of any of the aspects, the therapeutic gene coding sequence comprises a first nucleotide sequence selected from the group consisting of SEQ ID NO: 14, SEQ ID 15, and SEQ ID NO: 19, and a second nucleotide sequence selected from the group consisting of SEQ ID NO: 17, SEQ ID NO: 18, and SEQ ID NO: 22.

In some embodiments of any of the aspect, the therapeutic gene coding sequence is:

pd27mut_IL12 (8239 bp)
(SEQ ID NO: 24)
TCGCGCGTTTCGGTGATGACGGTGAAAACCTCTGACACATGCAGCTCCCGGAGACGGTCACAGCTTGTCTGTAAG
CGGATGCCGGGAGCAGACAAGCCCGTCAGGGCGCGTCAGCGGGTGTTGGCGGGTGTCGGGGCTGGCTTAACTATG
CGGCATCAGAGCAGATTGTACTGAGAGTGCACCATATGCGGTGTGAAATACCGCACAGATGCGTAAGGAGAAAAT
ACCGCATCAGGCGCCATTCGCCATTCAGGCTGCGCAACTGTTGGGAAGGGCGATCGGTGCGGGCCTCTTCGCTAT
TACGCCAGCTGGCGAAAGGGGGATGTGCTGCAAGGCGATTAAGTTGGGTAACGCCAGGGTTTTCCCAGTCACGAC
GTTGTAAAACGACGGCCAGTGAATTCGATATCACTAGTATTTAAATAACGTTGAGACGTCCTTAATCGTCCCGAC
GCTAAACGGCCGCGCACACCGCAGCCGCACCCCGCGCTTATCCTCCAGTTCGCGTAGGACCGGCGGGTGGTTAAC
CAGGTCCGCAAAGTTGCGGAGCTCGGTAATCAGCGGAGGGGTATGGTGGGTGTCCTTGTATACCGCAAAGAAAAA
GCAGTGGATTGTGCCGCTGGTCTCACAGGAGGCGCGGACCAGGTAACTCCGCACGGCCACGCAAGCGGAGTCCGT
TTTGCTGGTGTGCATGGCCGTTTCGGCCTGCCAGGTGGCGTTGAGGCAGTAAGGGGGGGCCACGTGGGTTATGTC
CGGGGCCCGTAAGAACAGGTTGGTGAGGGGGGTCGCTGTCATAGTGCAAAAGGGGGGATGCGCCCGGGCGGGAAG
CCCCTAAGGGCACTATGACACCGGCCTTGGAGCGGGGACGGATTTATACGTTGGGTTAGTTCCCTCCGCCCACCC
AGGCCGTACGCCGGGCCCACCCCCGCCATCTGCCGTGACCCACGCCCCGCCGGCCATGAGCAAAGAAGGACAACA
CGTGGGGCGATTTGTTTGAAATGTTTTGTTTTTATTGTACCTAAAACAAGGAGTTGCAATGAAAATATTTGCCGT
GCACGTACGGGGGGGCGACGATGTGACTGGCCGTCAACTCGCAGACACGACTCGAACACTCCTGGCGGTGCGTGT
CTAGGATTTCGATCAGGCCCGCCATGCAGGCCCCGGGGAGGTAGAAATGCATCTTCTCTCCGACCCCGACACCAA
GGGTCGCGTAGTCGATCTCCGCGACGCCACGCTCGACGCGGTTGGCGAGCCTGGCCAGAATGACAAACACGAAGG
ATGCAATGTCCTTAATGTCCGCCAGACGCCGCCGCGAACACAGTTCGTCCAGGCCGCACAGGCCTCGCGCCTTCA
GGTAGCACTGGAGAAAGGGCCGCAGGCGCGTGGCGAGGTTATCCAGCACCGCCGCGGCCGTCCCGATAATGGGGT
CCTGGGGGCGCAGCGGCAGGTTGTGGTGGATGCACATCTTGCACCACGCCAGCGTCTCGTCGGCGGAGGCCAGCG
CCTCGATGAAATTTTCTTGGCGCAGCACGCAGTCGCGCATGGCCTTGGCGGTCGATGCGGCCCGAGGATTGCCGG
CAAAGTGCGATAGAGGCTCGGGCCGTGGGCGACCAAGGTTTCCCAGGAGACCCGTCTGGTCTCGGCGTCAAAGGG
CCCTCCTTGGCCCGCCAGCACCGGGGCCCAGGGGCTATTCGCGGCGGGAAACGGCTGCCCCCCAAAGGGGTCGTA
CATGACCTGTGCGCTGCGGCCGGATCGATCTTCAATATTGGCCATTAGCCATATTATTCATTGGTTATATAGCAT
AAATCAATATTGGCTATTGGCCATTGCATACGTTGTATCTATATCATAATATGTACATTTATATTGGCTCATGTC
CAATATGACCGCCATGTTGGCATTGATTATTGACTAGTTATTAATAGTAATCAATTACGGGGTCATTAGTTCATA
GCCCATATATGGAGTTCCGCGTTACATAACTTACGGTAAATGGCCCGCCTGGCTGACCGCCCAACGACCCCCGCC
CATTGACGTCAATAATGACGTATGTTCCCATAGTAACGCCAATAGGGACTTTCCATTGACGTCAATGGGTGGAGT
ATTTACGGTAAACTGCCCACTTGGCAGTACATCAAGTGTATCATATGCCAAGTCCGCCCCCTATTGACGTCAATG
ACGGTAAATGGCCCGCCTGGCATTATGCCCAGTACATGACCTTACGGGACTTTCCTACTTGGCAGTACATCTACG
TATTAGTCATCGCTATTACCATGGTGATGCGGTTTTGGCAGTACACCAATGGGCGTGGATAGCGGTTTGACTCAC
GGGGATTTCCAAGTCTCCACCCCATTGACGTCAATGGGAGTTTGTTTTGGCACCAAAATCAACGGGACTTTCCAA
AATGTCGTAATAACCCCGCCCCGTTGACGCAAATGGGCGGTAGGCGTGTACGGTGGGAGGTCTATATAAGCAGAG
CTCGTTTAGTGAACCGTCAGATCACTAGAAGCTTTATTGCGGTAGTTTATCACAGTTAAATTGCTAACGCAGTCA
GTGCTTCTGACACAACAGTCTCGAACTTAAGGCTAGAGTACTTAATACGACTCACTATAGGCTAGCCTCGAGATG
TGTCCTCAGAAGCTAACCATCTCCTGGTTTGCCATCGTTTTGCTGGTGTCTCCACTCATGGCCATGTGGGAGCTG
GAGAAAGACGTTTATGTTGTAGAGGTGGACTGGACTCCCGATGCCCCTGGAGAAACAGTGAACCTCACCTGTGAC
ACGCCTGAAGAAGATGACATCACCTGGACCTCAGACCAGAGACATGGAGTCATAGGCTCTGGAAAGACCCTGACC
ATCACTGTCAAAGAGTTTCTAGATGCTGGCCAGTACACCTGCCACAAAGGAGGCGAGACTCTGAGCCACTCACAT
CTGCTGCTCCACAAGAAGGAAAATGGAATTTGGTCCACTGAAATTTTAAAAAATTTCAAAAACAAGACTTTCCTG
AAGTGTGAAGCACCAAATTACTCCGGACGGTTCACGTGCTCATGGCTGGTGCAAAGAAACATGGACTTGAAGTTC
AACATCAAGAGCAGTAGCAGTTCCCCTGACTCTCGGGCAGTGACATGTGGAATGGCGTCTCTGTCTGCAGAGAAG
GTCACACTGGACCAAAGGGACTATGAGAAGTATTCAGTGTCCTGCCAGGAGGATGTCACCTGCCCAACTGCCGAG
GAGACCCTGCCCATTGAACTGGCGTTGGAAGCACGGCAGCAGAATAAATATGAGAACTACAGCACCAGCTTCTTC
ATCAGGGACATCATCAAACCAGACCCGCCCAAGAACTTGCAGATGAAGCCTTTGAAGAACTCACAGGTGGAGGTC
AGCTGGGAGTACCCTGACTCCTGGAGCACTCCCCATTCCTACTTCTCCCTCAAGTTCTTTGTTCGAATCCAGCGC
AAGAAAGAAAAGATGAAGGAGACAGAGGAGGGGTGTAACCAGAAAGGTGCGTTCCTCGTAGAGAAGACATCTACC
GAAGTCCAATGCAAAGGCGGGAATGTCTGCGTGCAAGCTCAGGATCGCTATTACAATTCCTCATGCAGCAAGTGG
GCATGTGTTCCCTGCAGGGTCCGATCCGGCGGCGGCGGGAGTGGCGGCGGGGGTTCTGGCGGAGGCCTCGCTAGC
GGTGGCTCCATGGTCAGCGTTCCAACAGCCTCACCCTCGGCATCCAGCAGCTCCTCTCAGTGCCGGTCCAGCATG
TGTCAATCACGCTACCTCCTCTTTTTGGCCACCCTTGCCCTCCTAAACCACCTCAGTTTGGCCAGGGTCATTCCA
GTCTCTGGACCTGCCAGGTGTCTTAGCCAGTCCCGAAACCTGCTGAAGACCACAGATGACATGGTGAAGACGGCC
AGAGAAAAACTGAAACATTATTCCTGCACTGCTGAAGACATCGATCATGAAGACATCACACGGGACCAAACCAGC
ACATTGAAGACCTGTTTACCACTGGAACTACACAAGAACGAGAGTTGCCTGGCTACTAGAGAGACTTCTTCCACA
ACAAGAGGGAGCTGCCTGCCCCCACAGAAGACGTCTTTGATGATGACCCTGTGCCTTGGTAGCATCTATGAGGAC
TTGAAGATGTACCAGACAGAGTTCCAGGCCATCAACGCAGCACTTCAGAATCACAACCATCAGCAGATCATTCTA
GACAAGGGCATGCTGGTGGCCATCGATGAGCTGATGCAGTCTCTGAATCATAATGGCGAGACTCTGCGCCAGAAA
CCTCCTGTGGGAGAAGCAGACCCTTACAGAGTGAAAATGAAGCTCTGCATCCTGCTTCACGCCTTCAGCACCCGC
GTCGTGACCATCAACAGGGTGATGGGCTATCTGAGCTCCGCCTGAGCGGCCGCTTCGAGCAGACATGATAAGATA
CATTGATGAGTTTGGACAAACCACAACTAGAATGCAGTGAAAAAAATGCTTTATTTGTGAAATTTGTGATGCTAT
TGCTTTATTTGTAACCATTATAAGCTGCAATAAACAAGTTAACAACAACAATTGCATTCATTTTATGTTTCAGGT
TCAGGGGGAGATGTGGGAGGTTTTTTAAAGCAAGTAAAACCTCTACAAATGTGGTAAAATCGATAAGGATCGATC
TCCAGGCTACACGTGGATTATCATGGTATTTTTCATTTACATATGACTATACATTTCAAATGGGCCTTGCACTCA
ACTCGTTTCCAGTTTGCATATGCCGTTATGCGCGAATAATGCCTGGATGTGACGTCATACGTCAAACAGGCGCCT
CTGGATCTCCTGCTCGTAGTGAAGCGCCACGAGCACCACCCCGGCCACCACGGCGATATAACACAATCGCATTGC
GATGCCCGACAGGATGATGGAACAACAGCGCCCGCAGACGCCCGACAGCCCCTTGGATCGCCCCGGGGCGGCGGC
CTTGTCTGCGTTCTTGGGGGCCGGGCCCCGCCGCAGAATACAATACAGCTCTGTCaggCCGATGGTGGAGACAAA
ACACCAGGTGGTGATGGTCAGAAACAGGGGGTATGTGATCGCACATGCCCCCCGGGATATGAAAGCGGTGCCgAC
GATGAGACCCACGGCCACAAAGCGTAGCATCAACTCGCAGCCTACGATGACCCCGATGGCGGGGCGGTGGTACAA
GAAGGTGACCGGGTCCGTCTCAAACAACTGAACCAGGTTTTGCCGCTGGACCGACAGCTCGCAGAGCAGGCGGGT
AATTTTCGTGTAGGGGTACTGCAGGAACACGCTCGATACGATGCGGCCTGCGTAGTTCAAGAGGTAGGAGGCCGG
GGCCACCATCTTGTGGGCGGGACTCACGACACCAAACATACATCGGCGTTGGTGGAGGGCGACGAACGCCAGATA
CAGGAACCACCCTACGACCACCAGACGCACCCGTGTGTACCATAGGGTCTCCAGACAGTTAACTGCCTCGTGGAC
GTTCATGATCCGACGATTCGTGGCGTCGGGTGGGACCTGGAAGGGCACGACCCTACCCGCGATAAGATTGGCGTA
GCAGATATGGGCGTGGTTGCGCCAGCCCCCGTTGGGGGGGTGTGTCGGGGCCCCCAGAAACAATAGGGTCTGGTT
CATTTTCATCCACACGAGGGCGGTGTCGTTGTTGGTGCCGGTGGGGCGTACCGCGTAAATACATCGGTGCAGCGG
ACTGGCACCGAAGACGGTGTACCACACGAGCACGAGGCCGTACGCCGTTATCAAGACGACGGTTGAGAGGTGCTG
CAGGGAACGGACGGCGAGCATGGCGTGCCGGCGTCAATGGTAAACAGCGTGTGCAGGCGGTTGCTGTCGCATTTG
GCGGCAAAGCACTGCTGACACAAGGACACGCACAGGCGGTTGTTGGCCCCGACGCTCAGCGCGACGAATGTCCGC
GCCGTGGCGCGACTCGCCCGGCCGTGCTTAAAGCGCAGACACGACAGGCAACGTTATTTAATACTAGTGATATCA
AGCTTGGCGTAATCATGGTCATAGCTGTTTCCTGTGTGAAATTGTTATCCGCTCACAATTCCACACAACATACGA
GCCGGAAGCATAAAGTGTAAAGCCTGGGGTGCCTAATGAGTGAGCTAACTCACATTAATTGCGTTGCGCTCACTG
CCCGCTTTCCAGTCGGGAAACCTGTCGTGCCAGCTGCATTAATGAATCGGCCAACGCGCGGGGAGAGGCGGTTTG
CGTATTGGGCGCTCTTCCGCTTCCTCGCTCACTGACTCGCTGCGCTCGGTCGTTCGGCTGCGGCGAGCGGTATCA
GCTCACTCAAAGGCGGTAATACGGTTATCCACAGAATCAGGGGATAACGCAGGAAAGAACATGTGAGCAAAAGGC
CAGCAAAAGGCCAGGAACCGTAAAAAGGCCGCGTTGCTGGCGTTTTTCCATAGGCTCCGCCCCCCTGACGAGCAT
CACAAAAATCGACGCTCAAGTCAGAGGTGGCGAAACCCGACAGGACTATAAAGATACCAGGCGTTTCCCCCTGGA
AGCTCCCTCGTGCGCTCTCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCTCCAAGCTGGGCTGTGTG
CACGAACCCCCCGTTCAGCCCGACCGCTGCGCCTTATCCGGTAACTATCGTCTTGAGTCCAACCCGGTAAGACAC
GACTTATCGCCACTGGCAGCAGCCACTGGTAACAGGATTAGCAGAGCGAGGTATGTAGGCGGTGCTACAGAGTTC
TTGAAGTGGTGGCCTAACTACGGCTACACTAGAAGAACAGTATTTGGTATCTGCGCTCTGCTGAAGCCAGTTACC
TTCGGAAAAAGAGTTGGTAGCTCTTGATCCGGCAAACAAACCACCGCTGGTAGCGGTGGTTTTTTTGTTTGCAAG
CAGCAGATTACGCGCAGAAAAAAAGGATCTCAAGAAGATCCTTTGATCTTTTCTACGGGGTCTGACGCTCAGTGG
AACGAAAACTCACGTTAAGGGATTTTGGTCATGAGATTATCAAAAAGGATCTTCACCTAGATCCTTTTAAATTAA
AAATGAAGTTTTAAATCAATCTAAAGTATATATGAGTAAACTTGGTCTGACAGTTACCAATGCTTAATCAGTGAG
GCACCTATCTCAGCGATCTGTCTATTTCGTTCATCCATAGTTGCCTGACTCCCCGTCGTGTAGATAACTACGATA
CGGGAGGGCTTACCATCTGGCCCCAGTGCTGCAATGATACCGCGAGACCCACGCTCACCGGCTCCAGATTTATCA
GCAATAAACCAGCCAGCCGGAAGGGCCGAGCGCAGAAGTGGTCCTGCAACTTTATCCGCCTCCATCCAGTCTATT
AATTGTTGCCGGGAAGCTAGAGTAAGTAGTTCGCCAGTTAATAGTTTGCGCAACGTTGTTGCCATTGCTACAGGC
ATCGTGGTGTCACGCTCGTCGTTTGGTATGGCTTCATTCAGCTCCGGTTCCCAACGATCAAGGCGAGTTACATGA
TCCCCCATGTTGTGCAAAAAAGCGGTTAGCTCCTTCGGTCCTCCGATCGTTGTCAGAAGTAAGTTGGCCGCAGTG
TTATCACTCATGGTTATGGCAGCACTGCATAATTCTCTTACTGTCATGCCATCCGTAAGATGCTTTTCTGTGACT
GGTGAGTACTCAACCAAGTCATTCTGAGAATAGTGTATGCGGCGACCGAGTTGCTCTTGCCCGGCGTCAATACGG
GATAATACCGCGCCACATAGCAGAACTTTAAAAGTGCTCATCATTGGAAAACGTTCTTCGGGGCGAAAACTCTCA
AGGATCTTACCGCTGTTGAGATCCAGTTCGATGTAACCCACTCGTGCACCCAACTGATCTTCAGCATCTTTTACT
TTCACCAGCGTTTCTGGGTGAGCAAAAACAGGAAGGCAAAATGCCGCAAAAAAGGGAATAAGGGCGACACGGAAA
TGTTGAATACTCATACTCTTCCTTTTTCaATATTATTGAAGCATTTATCAGGGTTATTGTCTCATGAGCGGATAC
ATATTTGAATGTATTTAGAAAAATAAACAAATAGGGGTTCCGCGCACATTTCCCCGAAAAGTGCCACCTGACGTC
TAAGAAACCATTATTATCATGACATTAACCTATAAAAATAGGCGTATCACGAGGCCCTTTCGTC.

It is noted that the first and the second domains in the fusion protein can be positioned in any desired position in the fusion protein. For example, the first domain can be at the N-terminus of the second domain. In other words, the C-terminus of the first domain is linked to the N-terminus of the second domain. In some other embodiments, the first domain can be at the C-terminus of the second domain. In other words, the N-terminus of the first domain is linked to the C-terminus of the second domain. In some still other embodiments, the N-terminus of the first domain is linked to the N-terminus of the second domain or the C-terminus of the first domain is linked to the C-terminus of the second domain.

In some embodiments of any of the aspects, the fusion protein further comprises a linker between the first and second domain. As used herein, the term “linker” generally refers to a molecular entity that can directly or indirectly connect at two parts of a composition, e.g., the p35 to the p40 domains of IL-12. The linker can be a single peptide bond (e.g., the domains are linked directly to each other) or a peptide linker containing one or more amino acid residues (e.g. with an intervening amino acid or amino acid sequence, such as a peptide of one, two, three, four, five, six, seven, eight, nine, ten or more amino acids between the p35 domain and the p40 domain.

For example, the p35 and the p40 domain are linked via a peptide linker. The term “peptide linker” as used herein denotes a peptide with amino acid sequences, which is in some embodiments of synthetic origin. It is noted that peptide linkers may affect folding of a given fusion protein, and may also react/bind with other proteins, and these properties can be screened for by known techniques. A peptide linker can comprise 1 amino acid or more, 2 amino acids or more, 3 amino acids or more, 4 amino acids or more, 5 amino acids or more, 10 amino acids or more, 15 amino acids or more, 20 amino acids or more and beyond. Conversely, a peptide linker can comprise less than less than 20 amino acids, less than 15 amino acids, less than 10 amino acids or less than 5 amino acids.

In some embodiments of the various aspects described herein, the peptide linker comprises from about 2 amino acids to about 10 amino acids. For example, the peptide linker can comprise 2, 3, 4, 5, 6, 7, 7, 8, 9 or 10 amino acids.

Exemplary peptide linkers include those that consist of glycine and serine residues, the so-called Gly-Ser polypeptide linkers. As used herein, the term “Gly-Ser polypeptide linker” refers to a peptide that consists of glycine and serine residues. In some embodiments of the various aspects described herein, the peptide linker comprises the amino acid sequence (GlyₓSer)ₙ, where x is 2, 3, 4 or 5, and n is 1, 2, 3, 4, 5, 6, 7, 8, 9 or 10 (SEQ ID No: 25).

In some embodiments, the linker is a GlySer linker. In some embodiments, the linker comprises the amino acid sequence, (Gly-Gly-Gly-Gly-Ser)ₙ, where n is the copy number from 1 to 10 (SEQ ID NO: 26). In some embodiments of any of the aspects, the linker comprises the amino acid sequence: GGGGSGGGGSGGGLASGGS (SEQ ID NO: 27). Accordingly, in some embodiments of any of the aspects, the therapeutic gene coding sequence comprises a nucleotide sequence encoding the amino acid sequence of SEQ ID NO: 26. For example, the therapeutic gene coding sequence comprises the nucleotide sequence:
GGCGGCGGCGGGAGTGGCGGCGGGGGTTCTGGCGGAGGCCTCGCTAGCGGTGGCTCC (SEQ ID NO: 28).

Linkers can be configured according to a specific need, e.g., based on at least one of the following characteristics. In some embodiments of any of the aspects, the linker can be configured to have a sufficient length and flexibility such that it can allow for a cleavage at a target site. In some embodiments of any of the aspects, the linker can be configured to allow multimerization of the fusion protein domains provided herein. In some embodiments of any of the aspects, linkers can be configured to facilitate expression and purification of the fusion proteins provided herein.

In some embodiments of any of the aspects described herein, the linker is a flexible linker. As used herein, a “flexible linker” is a linker which does not have a fixed structure (secondary or tertiary structure) in solution and is therefore free to adopt a variety of conformations. Generally, a flexible linker has a plurality of freely rotating bonds along its backbone. In contrast, a rigid linker is a linker which adopts a relatively well-defined conformation when in solution. Rigid linkers are therefore those which have a particular secondary and/or tertiary structure in solution.

In some embodiments of any of the aspects, the nucleic acid sequence encoding the linker does not comprise an internal ribosome entry site (IRES).

### Methods of Preparing HSV-IL12 Compositions and Assaying for an Immune Response

The compositions provided herein can be prepared by any method known in the art. For example, by those described in Akhrameyeva et al, J. Virol. 55:5036-47 (2011); Lu, et al, J. Invest. Dermatol. 729: 1174-84 (2009); and Yao, et al, Hum. Gene Ther. 70:1811-8 (1999), which are incorporated herein by reference in their entireties.

The replication-defective oncolytic HSV1 (e.g., d106S-IL12) provided herein can be prepared by standard molecular cloning. For example, the IL-12 can be cloned into a shuttle plasmid (e.g., pd27B) that contains flanking homology arms for the U_{L}54 viral locus. Subsequently, cells can be transfected with the replication-defective oncolytic HSV DNA and the IL-12 plasmid to generate the replication-defective oncolytic HSV provided herein. Following generation of stocks of the recombinant virus provided herein from complementing cells, cells can be infected with virus at various multiplicities of infection (MOIs). Supernatants can then be collected and the presence or levels of the fusion protein can be assayed by methods known in the art.

In one aspect, the compositions provided herein elicit an immune response in a subject. In another aspect, the composition provided herein decreases tumor size and/or prevents metastasis of a tumor.

Assays for determining whether the compositions provided herein elicit an immune response in a subject or produce immunostimulatory activity in a cell or population thereof are known in the art. Immunostimulatory activity can be determined, for example, by detecting and measuring the levels of cytokines, chemokines, and/or interferon production in a biological sample (e.g, serum or tumor microenvironment).

Methods for detecting, measuring, and determining the levels of interferons and cytokines in a biological sample are known in the art. Interferon and cytokine polypeptide levels can be detected, for example, via immunoassay. ThermoFisher Scientific sells an ELISA-based kit for measuring human interferon gamma levels—see Catalog # 29-8319-65. IFN gene expression can also be detected. Methods of measuring gene expression are known in the art, e.g., PCR, microarrays, and immunodetection methods, such as Western blotting and immunocytochemistry, among others. For example, Quantitative reverse transcription polymerase chain reaction (qPCR) analysis can be performed using kits and arrays commercially available from, e.g., Applied Biosystems™—see Applied Biosystems® TaqMan® Array Human Interferon Pathway, catalog #4414154. See also, de Veer MJ et al. Functional classification of interferon-stimulated genes identified using microarrays. J Leukoc Biol. (2001) 69:912-20, which is incorporated herein by reference in its entirety.

Antibodies specific for a class of polypeptides (e.g., IFN-γ, IL-6, G-CSF, and CXCL2) are known in the art and can be used in immunohistochemistry, immunofluorescence, and Western Blotting, e.g., commercially available from Abcam™.

Interferon and cytokine levels and activity can also be determined using a reporter assay or a bioassay. See, e.g., Rees et al. J Immunol Methods, (2018), incorporated herein by reference in its entirety.

Viral infection assays can also be used to determine the effect of the Recombinant virus compositions provided herein. For example, the level of cell death in a population of cells after infection with a virus can be compared relative to a control population of cells. See, e.g., Barber et al. Host defense, viruses and apoptosis. Cell Death Differ 8, 113-126, (2001); and Liu, S. et al. Science 347, (2015), and which are incorporated herein by reference in its entirety.

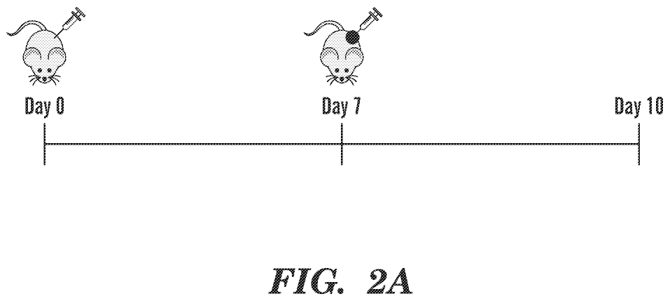

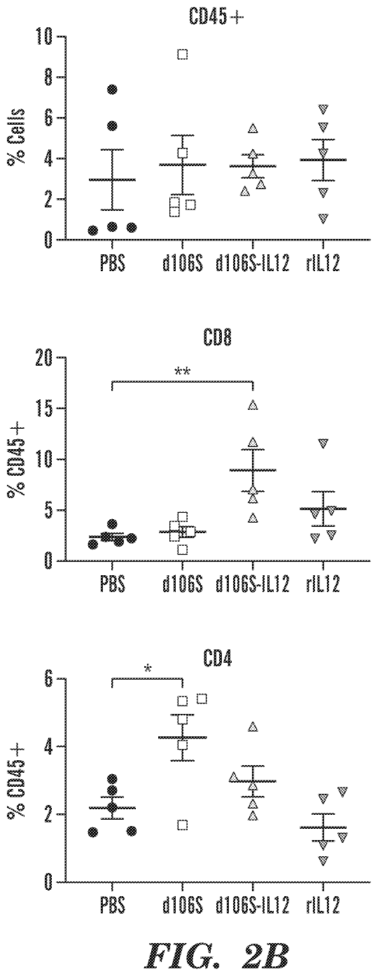

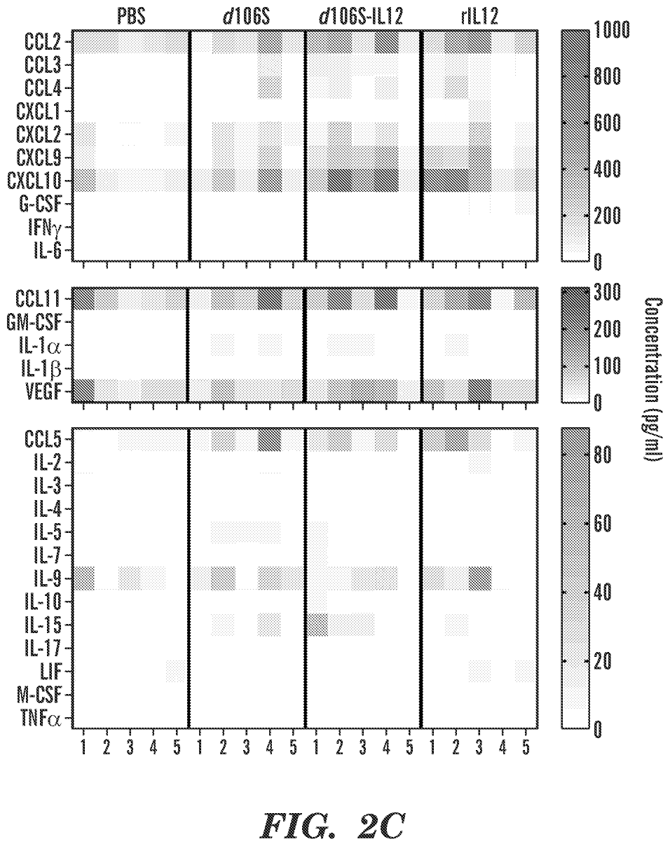

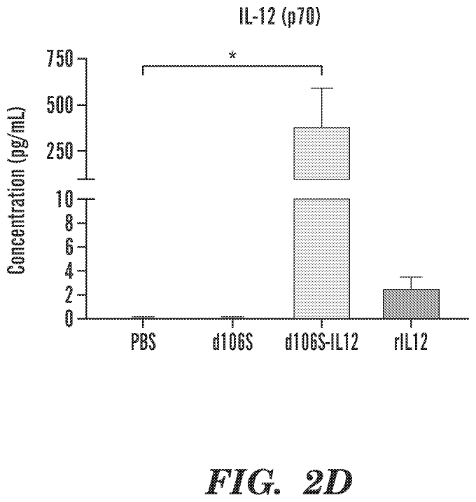

In addition, relevant animal models and human in vitro cell models can also be used to detect cytokine, chemokine, and interferon production directly or indirectly and to measure tumor cell size. The working examples provide methods of detecting tumor cell killing and the presence of immunostimulatory molecules within the tumor microenvironment in animal models injected with the recombinant virus provided herein compared with an appropriate control animal model (see, e.g., FIGS. 2-5). For example, flow cytometry and gene expression assays can be performed on isolated tumors from the animal models to measure the levels of biomarkers associated with an immune response or tumor cell killing by the host immune cells. Biomarkers for an immune response can include but are not limited to CD8, CD4, CD45, melanocyte-specific TRP1-tetramer, IFN-γ, CXCL10, CXCL9, CXCL2, G-CSF, GM-CSF, M-CSF, CCL2, CCL3, CCL4, CCL5, IL-β, IL-2, 3, 4, 5, 6, 7, 9, 10, 12, 15 and 17. See also, Ribas et al. Cell (2017), 170(6):1109-1119.e10, incorporated herein by reference in its entirety.

### Pharmaceutical and Anti-Cancer Vaccine Compositions

The recombinant virus compositions provided herein can further comprise formulating the recombinant virus provided herein with a pharmaceutically acceptable carrier.

Such formulations exploit the recombinant virus compositions as described herein to provide an adjuvant effect, e.g., when the formulation is administered as a vaccine, that promotes tumor cell killing by the host immune system.

The recombinant virus compositions provided herein will be used to immunize individual and/or patients typically by injection as an anti-cancer vaccine. For clinical use, administration of the compositions provided herein include formulation into pharmaceutical compositions or pharmaceutical formulations for parenteral administration, e.g., intravenous; mucosal, e.g., intranasal; ocular, or other mode of administration. The compositions described herein can be administered along with any pharmaceutically acceptable carrier compound, material, or composition which results in an effective treatment in the subject. Thus, a pharmaceutical formulation for use in the methods described herein can contain the recombinant virus composition provided herein in combination with one or more pharmaceutically acceptable ingredients. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, a recombinant virus composition provided herein. Each carrier must be “acceptable” in the sense of being compatible with the other ingredients of the formulation and not injurious to the patient. The terms “excipient,” “carrier,” “pharmaceutically acceptable carrier” or the like are used interchangeably herein.

The recombinant virus compositions provided herein can be formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) transdermally; (3) transmucosally; (4) via bronchoalveolar lavage.

In some embodiments, the compositions described herein comprise a particle or polymer-based vehicle. Exemplary particle or polymer-based vehicles include, but are not limited to, nanoparticles, microparticles, polymer microspheres, or polymer-drug conjugates.

In one embodiment of any of the aspects, the compositions described herein further comprise a lipid vehicle. Exemplary lipid vehicles include, but are not limited to, liposomes, phospholipids, micelles, lipid emulsions, and lipid-drug complexes.

In some embodiments, the recombinant virus compositions provided herein are formulated in a composition comprising micelles, amphiphilic carriers, polymers, cyclodextrins, liposomes, and encapsulation devices.

Microemulsification technology can improve bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing absorption to the lymphatic system instead of the circulatory system, which thereby bypasses the liver, and prevents destruction of the compounds in the hepatobiliary circulation.

In some embodiments, the recombinant virus compositions provided herein can be formulated with an amphiphilic carrier. Amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

In some embodiments, the Recombinant virus compositions provided herein can be formulated with hydrophilic polymers. Hydrophilic polymers are water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. Other hydrophilic polymers which may be suitable include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a pharmaceutical composition as described herein comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

In certain embodiments, a pharmaceutical compositions described herein is formulated as a liposome. Liposomes can be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic DD, Liposomes from physics to applications, Elsevier Science Publishers BV, Amsterdam, 1993.

Therapeutic formulations of the compositions as provided herein can be prepared for storage by mixing the recombinant virus having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Vaccine or other pharmaceutical compositions comprising a recombinant virus composition provided herein can contain a pharmaceutically acceptable salt, typically, e.g., sodium chloride, and preferably at about physiological concentrations. The formulations of the vaccine or other pharmaceutical compositions described herein can contain a pharmaceutically acceptable preservative. In some embodiments, the preservative concentration ranges from 0.1 to 2.0%, typically v/v. Suitable preservatives include those known in the pharmaceutical arts. Benzyl alcohol, phenol, m-cresol, methylparaben, and propylparaben are examples of preservatives. The formulations of the vaccine or other pharmaceutical compositions described herein can include a pharmaceutically acceptable surfactant at a concentration of 0.005 to 0.02%.

Therapeutic pharmaceutical compositions described herein can also contain more than one active compound as necessary for the particular indication being treated, preferably those with complementary activities that do not adversely affect each other.

Vaccine compositions

In some embodiments in which the recombinant virus compositions provided herein are formulated for use in or with a vaccine, the vaccine composition can be formulated with the recombinant virus as an adjuvant. In other embodiments the vaccine composition can be formulated with the recombinant virus composition provided herein and an additional adjuvant, e.g., as known in the art.

As used herein in the context of immunization, immune response and vaccination, the term “adjuvant” refers to any substance than when used in combination with a specific antigen produces a more robust immune response than the antigen alone. When incorporated into a vaccine formulation, an adjuvant acts generally to accelerate, prolong, or enhance the quality of specific immune responses to the vaccine antigen(s).

Adjuvants typically promote the accumulation and/or activation of accessory cells or factors to enhance antigen-specific immune responses and thereby enhance the efficacy of vaccines, i.e., antigen-containing or encoding compositions used to induce protective immunity against the antigen.

Adjuvants, in general, include adjuvants that create a depot effect, immune-stimulating adjuvants, and adjuvants that create a depot effect and stimulate the immune system. An adjuvant that creates a depot effect is an adjuvant that causes the antigen to be slowly released in the body, thus prolonging the exposure of immune cells to the antigen. This class of adjuvants includes but is not limited to alum (e.g., aluminum hydroxide, aluminum phosphate); emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720; AirLiquide, Paris, France); MF-59 (a squalene-in-water emulsion stabilized with Span 85 and Tween 80; Chiron Corporation, Emeryville, Calif.); and PROVAX™ (an oil-in-water emulsion containing a stabilizing detergent and a micelle-forming agent; DEC Pharmaceuticals Corporation, San Diego, Calif.).

An immune-stimulating adjuvant is an adjuvant that causes activation of a cell of the immune system. It may, for instance, cause an immune cell to produce and secrete cytokines and interferons. This class of adjuvants includes but is not limited to saponins purified from the bark of the Q. saponaria tree, such as QS21 (a glycolipid that elutes in the 21st peak with HPLC fractionation; Aquila Biopharmaceuticals, Inc., Worcester, Mass.); poly[di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). This class of adjuvants also includes CpG DNA.

Adjuvants that create a depot effect and stimulate the immune system are those compounds which have both of the above-identified functions. This class of adjuvants includes but is not limited to ISCOMS (immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxypropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

The active ingredients of the pharmaceutical compositions described herein can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

In some embodiments, sustained-release preparations can be used. Suitable examples of sustained-release preparations include semipermeable matrices of solid hydrophobic polymers containing a tumor antigen or fragment thereof in which the matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated, the antigen or fragment thereof can remain in the body for a long time, denature, or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S- bond formation through thio-disulfide interchange, stabilization can be achieved by modifying sulfhydryl residues, lyophilizing from acidic solutions, controlling moisture content, using appropriate additives, and developing specific polymer matrix compositions.

### Methods of Treatment

In one aspect, the compositions and methods provided herein can be used in the treatment of cancer. In another aspect, provided herein is a method of eliciting an immune response in a subject, the method comprising: administering to said subject the recombinant virus provided herein. In another aspect, provided herein is a method of treating cancer, the method comprising: administering to the subject in need thereof the recombinant virus provided herein.

As used herein, the term “cancer” refers to a hyperproliferation of cells that exhibit a loss of normal cellular control that results in unregulated growth, lack of differentiation, local tissue invasion, and metastasis. The methods and compositions described herein can be used for the treatment of solid tumors or non-solid tumors, such as leukemia, blood cell cancers, and the like. Solid tumors can be found in bones, muscles, the brain, or organs, and can be sarcomas or carcinomas. Where the methods and compositions described herein can overcome barriers of tumor treatment, including, but not limited to barriers to treatment or inhibition of metastases, it is contemplated that aspects of the technology described herein can be used to treat all types of solid and non-solid tumor cancers, including cancers not listed in the instant specification. The compositions and methods described herein, without limitation, include methods of treating cancer, methods of inhibiting metastases, and methods of inducing an anti-tumor immune response.

Non-limiting examples of cancer include but are not limited to, carcinoma, lymphoma, blastoma, sarcoma, and leukemia. More particular examples of such cancers include, but are not limited to basal cell carcinoma, biliary tract cancer; bladder cancer; bone cancer; brain and CNS cancer; breast cancer; cancer of the peritoneum; cervical cancer; cholangiocarcinoma; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; teratocarcinoma; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), tumors of primitive origins and Meigs' syndrome.

In one embodiment, the cancer is a cancer that is deficient in interferon-γ sensing, or has a loss or reduction of interferon-γ sensing. In one embodiment, the cancer is a cancer that results from a deficiency of interferon-γ sensing, or a loss or reduction of interferon-γ sensing. In one embodiment, a loss or reduction is at least 10%, 20%, 30%, 40%, 50%, 60%, 70%, 80%, 90%, 95%, 99%, or more of loss or reduction of interferon-γsensing as compared to a suitable control (i.e., an otherwise identical cell that does not have a loss or reduction of interferon-γ sensing).

In one embodiment, the cancer is deficient in a direct antigen presentation pathway. Cancer cells frequently produce abnormal proteins known as tumor antigens. These abnormal tumor proteins mark cancer cells as “non-self” for recognition by the immune system. However, some cancer cells do not produce these tumor antigens and are referred to as deficient in direct antigen presentation herein. This allows these cancer cells to escape the immune responses that ordinarily prevent the development of malignant tumors. Without wishing to be bound by a theory, deficient direct antigen presentation may be due to down regulation of major histocompatibility class (MHC) I expression, allowing antigen to go unrecognized, and/or lack of co-stimulatory signals needed for antigen presentation, i.e., loss or alteration of the MHC molecule.

By “reduce” or “inhibit” in terms of the cancer treatment methods described herein is meant the ability to cause an overall decrease preferably of 20% or greater, 30% or greater, 40% or greater, 45% or greater, more preferably of 50% or greater, of 55% or greater, of 60% or greater, of 65% or greater, of 70% or greater, and most preferably of 75% or greater, 80% or greater, 85% or greater, 90% or greater, or 95% or greater, for a given parameter or symptom. Reduce or inhibit can refer to, for example, the presence or size of metastases or micrometastases, the size of the primary tumor, the presence or the size of the dormant tumor, etc. A patient or subject who is being treated for a cancer or tumor is one who a medical practitioner has diagnosed as having such a condition. Diagnosis can be by any suitable means.

In some embodiments of any of the aspects, the recombinant virus provided herein reduces tumor mass and/or volume in a subject. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., Breast (Edinburgh, Scotland) (2003) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of therapy or upon surgical removal of residual tumor cells and/or the tumor bed.

Additional criteria for evaluating the response to anti-cancer therapies (e.g., administration of the recombinant virus or cell contacted with said recombinant virus provided herein) are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

In some embodiments of any of the aspects, the recombinant virus provided herein is administered in combination with one or more anti-cancer agent or therapies. The therapy may be any anti-cancer therapy including, but not limited to, chemotherapy, radiation therapy, immunotherapy, small molecule inhibitors, shRNA, hormonal, and combinations thereof.

Non-limiting examples of chemotherapeutic agents can include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB.); inhibitors of PKC-alpha, Raf, H-Ras, EGFR (e.g., erlotinib (TARCEVA®)) and VEGF-A that reduce cell proliferation and pharmaceutically acceptable salts, acids or derivatives of any of the above. In addition, the methods of treatment can further include the use of radiation or radiation therapy.

Chemotherapeutic agents as used herein encompass both chemical and biological agents. These agents function, for example, to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. In one embodiment, a chemotherapeutic agent is an agent of use in treating neoplasms such as solid tumors. In one embodiment, a chemotherapeutic agent is a radioactive molecule. One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; Baltzer L, Berkery R (eds): Oncology Pocket Guide to Chemotherapy, 2nd ed. St. Louis, Mosby-Year Book, 1995; Fischer D S (ed): The Cancer Chemotherapy Handbook, 4th ed. St. Louis, Mosby-Year Book, 2003)).

### Dosage, Administration, Efficacy

### Dosage

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions, methods, and uses that exhibit large therapeutic indices are preferred. A therapeutically effective dose can be estimated initially from cell culture assays. Also, a dose can be formulated in animal models to achieve a circulating plasma concentration range that includes the IC50, which achieves a half-maximal inhibition of measured function or activity as determined in cell culture, or in an appropriate animal model. The effects of any particular dosage can be monitored by a suitable bioassay. The dosage can be determined by a physician and adjusted, as necessary, to suit observed effects of the treatment.

### Administration

Although certain routes of administration are provided in the foregoing description, according to the invention, any suitable route of administration of the vectors may be adapted, and therefore the routes of administration described above are not intended to be limiting. Routes of administration may include, but are not limited to, intravenous, regional artery infusion, oral, buccal, intranasal, inhalation, topical application to a mucosal membrane or injection, including intratumoral, intradermal, intrathecal, intracisternal, intralesional or any other type of injection. Administration can be effected continuously or intermittently and will vary with the subject and the condition to be treated. One of skill in the art would readily appreciate that the various routes of administration described herein would allow for the inventive vectors or compositions to be delivered on, in, or near the tumor or targeted cancer cells. One of skill in the art would also readily appreciate that various routes of administration described herein will allow for the vectors and compositions described herein to be delivered to a region in the vicinity of the tumor or individual cells to be treated. “In the vicinity” can include any tissue or bodily fluid in the subject that is in sufficiently close proximity to the tumor or individual cancer cells such that at least a portion of the vectors or compositions administered to the subject reach their intended targets and exert their therapeutic effects.

Prior to administration, the oncolytic viruses can be suspended in any pharmaceutically acceptable solution including sterile isotonic saline, water, phosphate buffered saline, 1,2-propylene glycol, polyglycols mixed with water, Ringer's solution, etc. The exact number of viruses to be administered is not crucial to the invention but should be an “effective amount,” i.e., an amount sufficient to cause cell lysis extensive enough to generate an immune response to released tumor antigens. Since virus is replicated in the cells after infection, the number initially administered will increase rapidly with time. Thus, widely different amounts of initially administered virus can give the same result by varying the time that they are allowed to replicate. In general, it is expected that the number of viruses (PFU) initially administered will be between 1×10⁶ and 1×10¹⁰.

### Efficacy

The effectiveness of a dosage, as well as the effectiveness of the overall treatment can be assessed by monitoring tumor size using standard imaging techniques over a period of days, weeks and/or months. A shrinkage in the size or number of tumors is an indication that the treatment has been successful. If this does not occur or continue, then the treatment can be repeated as many times as desired. In addition, treatment with virus can be combined with any other therapy typically used for solid tumors, including surgery, radiation therapy or chemotherapy. In addition, the procedure can be combined with methods or compositions designed to help induce an immune response.

As used herein, the term “therapeutically effective amount” is intended to mean the amount of vector which exerts oncolytic activity, causing attenuation or inhibition of tumor cell proliferation, leading to tumor regression. An effective amount will vary, depending upon the pathology or condition to be treated, by the patient and his or her status, and other factors well known to those of skill in the art. Effective amounts are easily determined by those of skill in the art. In some embodiments a therapeutic range is from 10³ to 10¹² plaque forming units introduced once. In some embodiments a therapeutic dose in the aforementioned therapeutic range is administered at an interval from every day to every month via the intratumoral, intrathecal, convection-enhanced, intravenous or intra-arterial route.

In vitro and animal model assays are provided herein which allow the assessment of a given dose of a recombinant virus composition.

### Some Selected Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g., cancer. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

The terms “decrease”, “reduce”, “reduction”, or “inhibit” are all used herein to mean a decrease by a statistically significant amount. In some embodiments, “reduce,” “reduction”, “decrease” or “inhibit” means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, a “subject” is a human or a non-human animal. Usually the non-human animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of diseases including diseases and disorders involving inappropriate immunosuppression. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors. In some embodiments, the subject has been diagnosed with or is suspected of having cancer.

As used herein, a “subject in need” of treatment for a particular condition can be a subject having that condition, diagnosed as having that condition, or at risk of developing that condition.

As used herein, the term “alteration” refers to any change in a native wild-type nucleic acid or amino acid sequence. Alterations can include, but are not limited to, insertions, deletions, mutations, substitutions, or molecular changes as compared to a reference sequence. Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. ligand-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to an assay known in the art or described below herein. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

As used herein, the term “DNA” is defined as deoxyribonucleic acid. The term “polynucleotide” is used herein interchangeably with “nucleic acid” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “operably linked,” as used herein, refers to the arrangement of various nucleic acid molecule elements relative to each other such that the elements are functionally connected and are able to interact with each other. Such elements may include, without limitation, a promoter, an enhancer, a polyadenylation sequence, one or more introns and/or exons, and a coding sequence of a gene of interest to be expressed. The nucleic acid sequence elements, when operably linked, can act together to modulate the activity of one another, and ultimately may affect the level of expression of the gene of interest, including any of those encoded by the sequences described above.

The term “vector,” as used herein, refers to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both of which are incorporated herein by reference). Additionally, the techniques described herein and demonstrated in the referenced figures are also instructive with regard to effective vector construction.

The terms “replication-defective oncolytic Herpes simplex virus 1 (HSV-1) recombinant virus” or “oncolytic HSV-1 vector” or refers to a genetically engineered HSV-1 virus corresponding to at least a portion of the genome of HSV-1 that is capable of infecting a target cell, replicating, and being packaged into HSV-1 virions. The genetically engineered virus comprises deletions and or mutations and or insertions of nucleic acid that render the virus oncolytic such that the engineered virus replicates in—and kills—tumor cells by oncolytic activity. The virus may be attenuated or non-attenuated. The virus may deliver a transgene-that differs from the HSV viral genome.

The term “promoter,” as used herein, refers to a nucleic acid sequence that regulates, either directly or indirectly, the transcription of a corresponding nucleic acid coding sequence to which it is operably linked. The promoter may function alone to regulate transcription, or, in some cases, may act in concert with one or more other regulatory sequences such as an enhancer or silencer to regulate transcription of the gene of interest. The promoter comprises a DNA regulatory sequence, wherein the regulatory sequence is derived from a gene, which is capable of binding RNA polymerase and initiating transcription of a downstream (3′-direction) coding sequence. A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one can position the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. Depending on the promoter used, individual elements can function either cooperatively or independently to activate transcription. The promoters described herein may or may not be used in conjunction with an “enhancer,” which refers to a cis-acting regulatory sequence involved in the transcriptional activation of a nucleic acid sequence, such as those for the genes, or portions or functional equivalents thereof, listed herein.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages may be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include, the HCMV immediate-early promoter, the beta-lactamase (penicillinase), lactose and tryptophan (trp) promoter systems.

A “gene,” or a “sequence which encodes” a particular protein, is a nucleic acid molecule which is transcribed (in the case of DNA) and translated (in the case of mRNA) into a polypeptide in vitro or in vivo when placed under the control of one or more appropriate regulatory sequences. A gene of interest can include, but is no way limited to, cDNA from eukaryotic mRNA, genomic DNA sequences from eukaryotic DNA, and even synthetic DNA sequences. A transcription termination sequence will usually be located 3′ to the gene sequence. Typically, a polyadenylation signal is provided to terminate transcription of genes inserted into a recombinant virus.

The term “ polypeptide ” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term “transgene” refers to a particular nucleic acid sequence encoding a polypeptide or a portion of a polypeptide to be expressed in a cell into which the nucleic acid sequence is inserted. The term “transgene” is meant to include (1) a nucleic acid sequence that is not naturally found in the cell (i.e., a heterologous nucleic acid sequence); (2) a nucleic acid sequence that is a mutant form of a nucleic acid sequence naturally found in the cell into which it has been inserted; (3) a nucleic acid sequence that serves to add additional copies of the same (i.e., homologous) or a similar nucleic acid sequence naturally occurring in the cell into which it has been inserted; or (4) a silent naturally occurring or homologous nucleic acid sequence whose expression is induced in the cell into which it has been inserted. A “mutant form” or “modified nucleic acid” or “modified nucleotide” sequence means a sequence that contains one or more nucleotides that are different from the wild-type or naturally occurring sequence, i.e., the mutant nucleic acid sequence contains one or more nucleotide substitutions, deletions, and/or insertions. In some cases, the gene of interest may also include a sequence encoding a leader peptide or signal sequence such that the transgene product may be secreted from the cell.

The term “oncolytic activity,” as used herein, refers to cytotoxic effects in vitro and/or in vivo exerted on tumor cells without any appreciable or significant deleterious effects to normal cells under the same conditions. The cytotoxic effects under in vitro conditions are detected by various means as known in prior art, for example, by staining with a selective stain for dead cells, by inhibition of DNA synthesis, or by apoptosis. Detection of the cytotoxic effects under in vivo conditions is performed by methods known in the art.

A “biologically active” portion of a molecule, as used herein, refers to a portion of a larger molecule that can perform a similar function as the larger molecule. Merely by way of non-limiting example, a biologically active portion of a promoter is any portion of a promoter that retains the ability to influence gene expression, even if only slightly. Similarly, a biologically active portion of a protein is any portion of a protein which retains the ability to perform one or more biological functions of the full-length protein (e.g. binding with another molecule, phosphorylation, etc.), even if only slightly.

As used herein, the term “administering,” refers to the placement of a therapeutic or pharmaceutical composition as disclosed herein into a subject by a method or route which results in at least partial delivery of the agent at a desired site. Pharmaceutical compositions comprising agents as disclosed herein can be administered by any appropriate route which results in an effective treatment in the subject.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

It is to be understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The invention described herein can be further described in any of the following numbered paragraphs.

1. A replication-defective oncolytic herpes simplex virus 1 (HSV-1) recombinant virus, comprising within its genome:
i. one or more therapeutic gene coding sequences,
ii. wherein the genome comprises at least one alteration in each of a gene encoding infected cell polypeptides (ICP) 4, a gene encoding ICP22, a gene encoding ICP27 and a gene encoding ICP47, and
iii. wherein the genome does not encode a functional ICP4, ICP22, ICP27 and ICP47 protein.

2. The recombinant virus of paragraph 1, wherein the therapeutic gene coding sequence is inserted in place of the U_{L}54 loci open reading frame 1 (ORF1).

3. The recombinant virus of any one of paragraphs 1-2, wherein the genome further comprises at least one alteration in an S component repeated sequence in a promoter between a gene encoding ICP4 and a gene encoding ICP22 or in a promoter between a gene encoding ICP4 and a gene encoding ICP47.

4. The recombinant virus of any one of paragraphs 1-3, wherein the genome lacks at least one Oct-1 site present in wild-type HSV-1 genome.

5. The recombinant virus of any one of paragraphs 1-4, wherein the genome lacks at least one Oct-1 site present in a promoter of a gene encoding ICP22 or a gene encoding ICP47.

6. The recombinant virus any one of paragraphs 1-5, wherein the genome lacks at least one (e.g., one, two or three) SP1 binding site present in wild-type HSV-1 genome.

7. The recombinant virus of any one of paragraphs 1-6, wherein the genome lacks at least one (e.g., one, two or three) SP1 binding site present in a promoter of a gene encoding ICP22 or a gene encoding ICP47.

8. The recombinant virus of any one of paragraphs 1-7, wherein the therapeutic gene coding sequence is a codon optimized sequence.

9. The recombinant virus of any one of paragraphs 1-8, wherein the therapeutic gene coding sequence does not comprise an internal ribosome entry site (IRES).

10. The recombinant virus of any one of paragraphs 1-9, wherein the virus comprises two or more therapeutic gene coding sequences within its genome.

11. The recombinant virus of any one of paragraphs 1-10, wherein the virus comprises two or more therapeutic gene coding sequences within its genome and wherein at least two of the therapeutic gene coding sequences are different.

12. The recombinant virus of any one of paragraphs 1-11, wherein the therapeutic gene coding sequence encodes a cytokine.

13. The recombinant virus of any one of paragraphs 1-12, wherein the therapeutic gene coding sequence encodes a heterodimeric cytokine.

14. The recombinant virus of any one of paragraphs 1-13, wherein the therapeutic gene coding sequence encodes interleukin 12 (IL-12), IL-2, IL-3, IL-4, IL-5, IL-6, IL-7, IL-9, IL-10, IL-12, IL-15, IL-17, IL-18, interferon (IFN)-γ, or any combinations thereof.

15. The recombinant virus of any one of paragraphs 1-14, wherein the therapeutic gene coding sequence encodes a non-fusion protein.

16. The recombinant virus of any one of paragraphs 1-15, wherein the therapeutic gene coding sequence encodes a fusion protein.

17. The recombinant virus of any one of paragraphs 1-16, wherein the therapeutic gene coding sequence encodes a fusion protein comprising a first domain and a second domain linked via a linker, wherein the nucleotide sequence encoding the linker does not comprise an IRES.

18. The recombinant virus of any one of paragraphs 1-17, wherein the therapeutic gene coding sequence encodes a fusion protein comprising a first domain and a second domain linked via a linker, wherein one of the first and second domain is a p40 subunit of IL-12 and the other domain is a p35 subunit of IL-12.

19. The recombinant virus of any one of paragraphs 1-18, wherein the therapeutic gene coding sequence comprises a nucleotide sequence selected from the group consisting of SEQ ID NO: 14-SEQ ID NO: 24 and any combinations thereof.

20. The recombinant virus of any one of paragraphs 1-19, wherein the therapeutic gene coding sequence comprises a nucleic acid sequence encoding an amino acid sequence selected from the group consisting of SEQ ID NO: 14, SEQ ID NO: 17, SEQ ID NO: 19, SEQ ID NO: 21, SEQ ID NO: 22, and any combinations thereof.

21. The recombinant virus of any one of paragraphs 1-20, wherein the recombinant virus has increased sensitivity to acyclovir relative to wild-type HSV-1.

22. A composition comprising the recombinant virus of any one of paragraphs 1-20.

23. A vaccine and/or immunomodulatory virus comprising the recombinant virus of any one of paragraphs 1-20.

24. A method of eliciting and/or modifying an immune response in a subject, the method comprising administering to said subject the recombinant virus of any one of paragraphs 1-20.

25. The method of any of paragraph 24, wherein the subject is diagnosed or has been diagnosed as having cancer.

26. A method of treating cancer, the method comprising: administering to the subject in need thereof the recombinant virus of any one of paragraphs 1-20.

27. The method of paragraph 25 or 26, wherein the cancer is a solid tumor, a benign tumor, or a malignant tumor.

28. The method of any one of paragraphs 25-27, wherein the cancer is selected from the group consisting of a carcinoma, a melanoma, a sarcoma, a germ cell tumor, and a blastoma.

29. The method of any one of paragraphs 25-28, wherein the cancer is metastatic.

### EXAMPLES

The following examples are provided by way of illustration not limitation.

### Example 1

### Immunotherapeutic Virus for Cancer

Provided herein is a non-replicating HSV virus, termed d106S, which can serve as a potentially safe immunotherapeutic and vector virus for the treatment of a variety of tumor types. This virus allows for expression of transgenes that could encode for immunostimulatory molecules, such as cytokines, checkpoint inhibitors, or tumor-associated antigens. The d106S was engineered as a vector to deliver interleukin (IL)-12 to the tumor microenvironment.

IL-12 is a potent cytokine capable of organizing a Thl response against tumors by enhancing the growth and cytotoxicity of NK cells, CD8+ and CD4+ T cells. However, due to the pleiotropic effects of IL-12, dose-limiting toxicities often become a barrier to effective treatment. Our replication-defective d106S virus may be beneficial for use with this cytokine to release a large burst of IL-12 locally within the tumor environment. Because the virus cannot replicate, further production of IL-12 would only be dependent on additional injections of the virus. the d106S-IL12 vector was engineered and found that its use initially drove CD8 infiltration into B16 tumors. However, with long-term production of IL-12 from the d106S-IL12 vector, tumors were infiltrated with tumor-antigen specific CD8s. The d106S-IL12 treatment led to prolonged survival when compared control and d106S-GFP treated mice and is capable of being used as an immunotherapeutic treatment for cancer.

HSV-1 d106S was designed to be a gene expression vector for transient expression of a trans-gene (Liu et al., 2009), but it also has been shown to have immunotherapeutic effects that led to tumor cell killing (12). HSV-1 recombinant viruses constructed to express IL-12 have been tested for oncolytic activity, but none were viruses designed to be gene expression vectors, and lower levels of IL-12 were likely expressed. In addition, a trans-gene expressing a fusion protein of the two IL-12 subunits was used to ensure equal expression levels of the two subunits. These features define the novel aspects of this recombinant immunotherapeutic virus.

### Results

Construction of a Replication-Defective HSV-1 Recombinant Expressing mIL-12

IL-12 is a heterodimeric cytokine composed of p35 and p40 subunits. An internal ribosome entry site (IRES) is one method use to express both proteins¹⁻³. However, efficient expression of the second gene product does not always occur in this system⁴. The ratio of p40 to p35 is important⁵, as homodimers of p40 antagonize the activity of IL-12^{6,7}. Therefore, an IL-12 fusion protein with a glycine-serine linker between the subunits was engineered as an immunotherapeutic agent. This fusion protein has been shown to be both stably secreted and bioactive⁸.

The murine IL-12 fusion protein (p40-GlySer-p35) was cloned into the shuttle plasmid pd27B⁹ which contains flanking homology arms for the U_{L}54 viral locus. Complementing cells were co-transfected with infectious d106S DNA and the pd27B-IL12. d106S has CMV-promoter-driven GFP expression within the UL54 locus (FIG. 1A), but upon homologous recombination with pd27B-IL12, GFP-negative plaques were isolated and purified.

Following generation of d106S-IL12 stocks from complementing cells, B16^{Nectin1} cells were infected with this virus at various multiplicities of infection (MOIs). After 24 hours of infection, supernatants were collected and IL-12 (p40) concentration was measured by ELISA. Even at the lowest MOI tested, d106S-IL12 produced abundant levels of cytokine: ˜9000 pg/ml per 10⁵ cells (FIG. 1B).

d106S Blunts Internal Interferon Response, but Stimulates External Response

The d106S vector has been previously shown to suppress the interferon response through its expression of the viral protein ICP0^{10,11}. However, it has also been seen that d106S infection of human melanoma cells stimulated plasmacytoid dendritic cells (pDCs) to become tumoricidal¹². To understand both the internal and external response to d106S and the new IL-12 producing vector, we infected B16^{Nectin1} cells with d106S or d106S-IL12 and co-cultured these cells with murine bone-marrow dendritic cells (BMDCs), or separately stimulated BMDCs with virus alone. mRNA expression was measured with a panel of ISGs. As shown previously^{10,11}, infection with d106S resulted in little interferon response within the melanoma cells. However, both co-culture of infected B16 with BMDCs and the sensing of free virus by BMDCs resulted in a robust external interferon response. Additionally, the secretion of IL-12 had an extra stimulatory effect (FIG. 1C, 1D).

Intratumoral Administration of d106S-IL12 Elicits an Initial CD8 Infiltration

After establishing productive IL-12 secretion in vitro in the B16^{Nectin1} cells (FIG. 1B), we analyzed the changes in tumor immune infiltrate induce by this virus in vivo. We established B16^{Nectin1} tumors orthotopically by subcutaneous injection into the flank of mice. After one week of tumor growth, mice were randomized and injected once intratumorally with either d106S(GFP) virus, d106S-IL12 virus, recombinant mIL-12 (0.1 μg), or saline (PBS) (FIG. 2A). The dosing for recombinant IL-12 was chosen to both avoid toxicity¹³, while also maintaining efficacy, at least for daily injections¹⁴. At three days following a single intratumoral injection (10 days post tumor challenge), tumors were removed, and immune infiltrate analyzed by flow cytometry.

The levels of CD8⁺ T-cells were significantly higher in tumors treated with d106S-IL12 compared with PBS control (FIG. 2B). Conversely, injection of recombinant mIL-12 resulted in a slight, but non-significant increase of CD8 cells over PBS treated mice. The d106 S virus did not stimulate a CD8 response, suggesting that the IL-12 secretion from d106s-IL12 was the cause. There was no difference in levels of CD4 T-cells in tumors treated with d106S-IL12 or recombinant mIL-12. However, there was a significant increase in CD4 T-cells in d106S-treated tumors. Levels of tumor-associated macrophages (TAMs) and granulocytic myeloid-derived suppressor cells (GrMDSCs) dropped slightly in d106S-IL12 tumors, while monocytic MDSCs (MoMDSCs) and CD103⁺ cross-presenting dendritic cells (DCs) were slightly elevated over control. Other immune cell subsets, such as B cells, were unchanged between PBS and d106S-IL12 treated tumors.

In addition to flow-cytometric analysis of these tumors, protein lysates were made from a portion of the tumors. A cytokine/chemokine multiplex panel was run on these protein lysates to determine concentrations of different chemokines within the tumor microenvironment (FIG. 2C).

Of note, the highest secreted cytokine in mice treated with d106S-IL12 was in fact IL-12 (FIG. 2D). Downstream targets of IL-12, such as IFNγ, were present at higher concentrations in d106S-IL12 treated tumors compared to control tumors, as were the T-cell chemoattractants CXCL10 and CXCL9. Cytokines slightly, yet significantly increased in the d106S-IL12 tumors were GM-CSF, M-CSF and IL-10. Many chemokines, such as CCL2, CCL3, CCL4, CXCL2, and CCL5 were highly increased in the d106S-IL12 treated microenvironment over control, though not to a significant extent. IL-10 and IL-5 were slightly, but significantly higher in d106S-IL12 treated tumors (FIG. 2C).

Intratumoral Administration of d106S-IL12 Induces an Antigen-Specific T Cell Response.

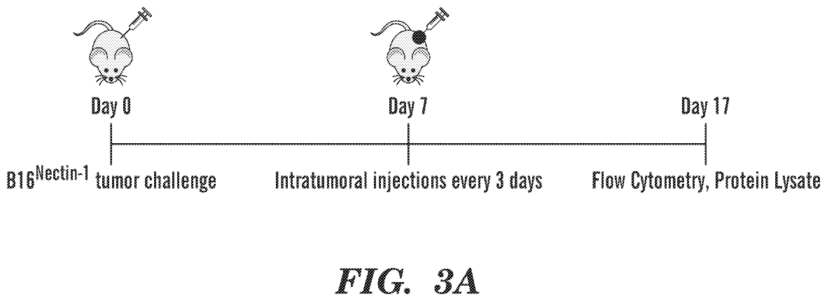

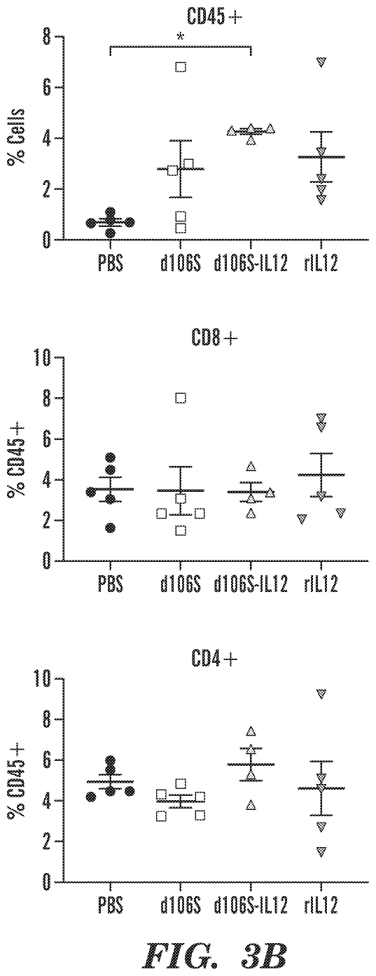

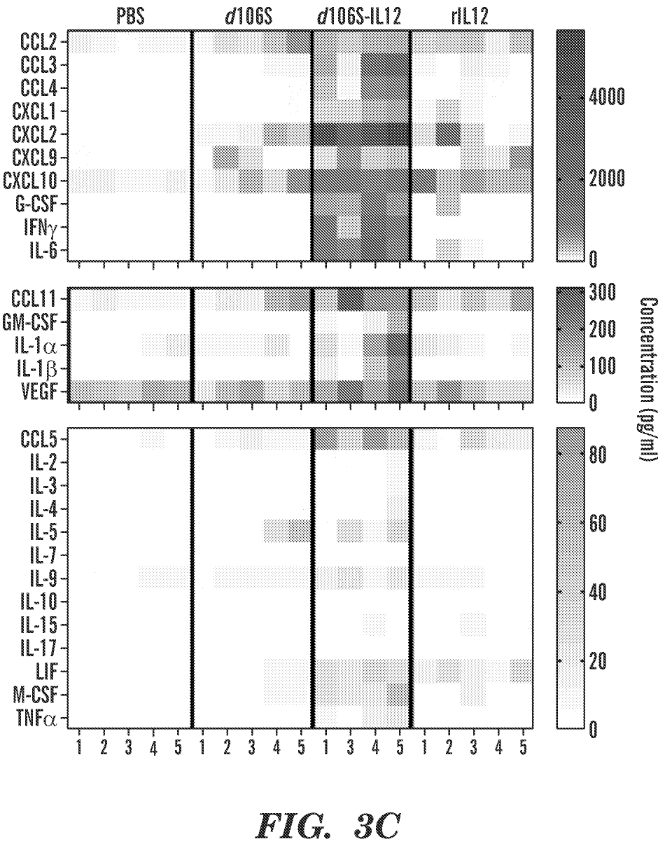

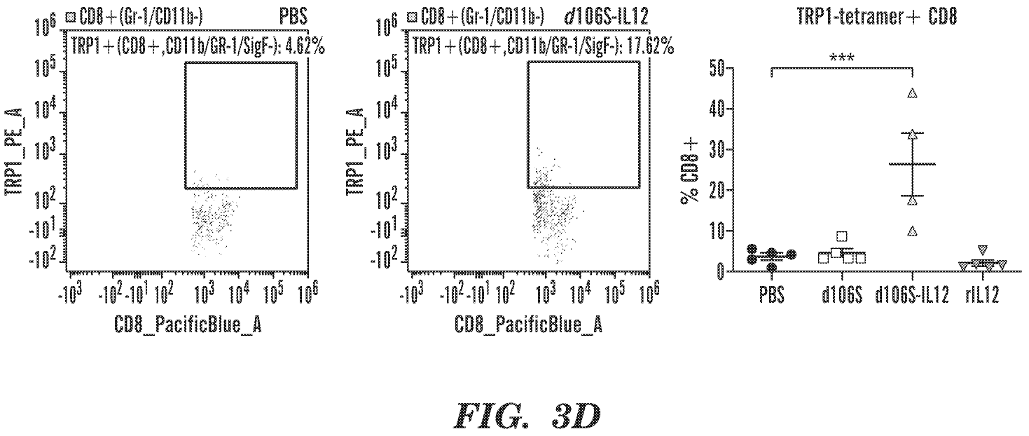

After discovering that d106S-IL12 virus stimulated initial CD8 T cell infiltration into the tumor accompanied by secretion of T cell and other immune cell chemoattracts, we chose to explore what changes happen to the tumor immune microenvironment at a later stage of tumor growth. As before, we established B16^{Nectin}1 tumors orthotopically by subcutaneous injection. After one week of tumor growth, mice were injected every three days (FIG. 3A). After a total of four injections, on Day 17 post tumor challenge, tumors were removed, and immune infiltrate analyzed by flow cytometry. One mouse in the d106S-IL12 group was not analyzed because its tumor was not visible by Day 17.

In contrast to Day 10, after one single injection at Day 7 (FIG. 2B), on Day 17, there was no significant increase in CD8⁺ T cells infiltrating tumors treated with d106S-IL12 (FIG. 3B). There was also no significant change in CD4 cells infiltrating d106S treated tumors (FIG. 3B), unlike at day 10 (FIG. 2B). However, staining with a melanocyte-specific TRP1-tetrameric showed significantly more TRP1-tetramer⁺ CD8⁺ cells within the tumors of mice treated with d106S-IL12, with levels reaching almost 50% in one tumor (FIG. 3D). Other subsets of immune cells were unchanged between groups, although levels of bulk CD45⁺ cells were significantly higher in d106S-IL12 treated tumors (FIG. 2B).

The same cytokine/chemokine multiplex panel run on Day 10 tumors was run on the Day 17 tumors. While levels of secreted IL-12 were still very high, almost all cytokines and chemokines had increased substantially since day 10 in mice treated with d106S-IL12. Those that increased included CXCL2, G-CSF, IL-6 and IFNγ, which were secreted an average of 40- to almost 100-fold more than day 10. Those cytokines that remained low and mostly unchanged since Day 10 included IL-2, 3, 4, 5, 7, 9, 10, 15, and IL-17 (FIG. 3C).

Injection of d106S-IL12 Protects Mice from Melanoma

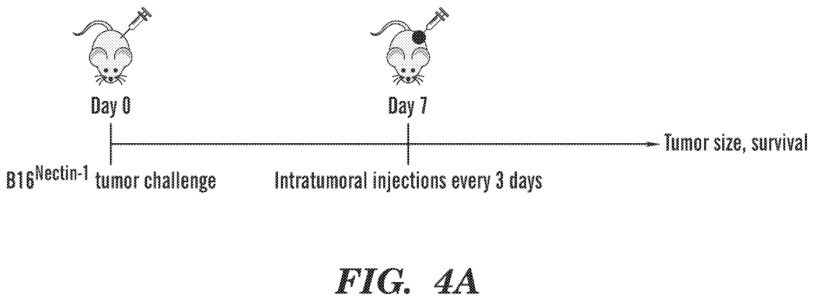

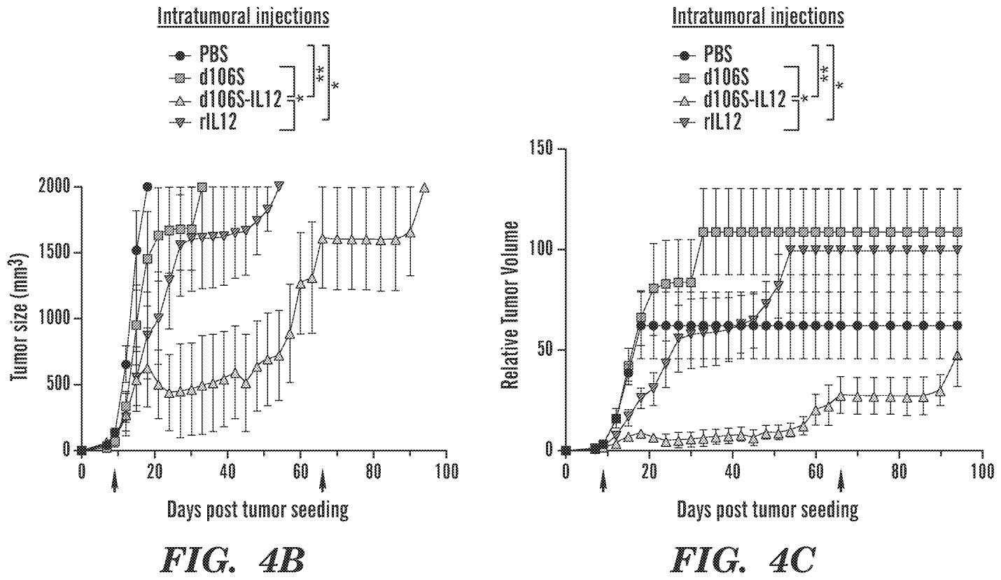

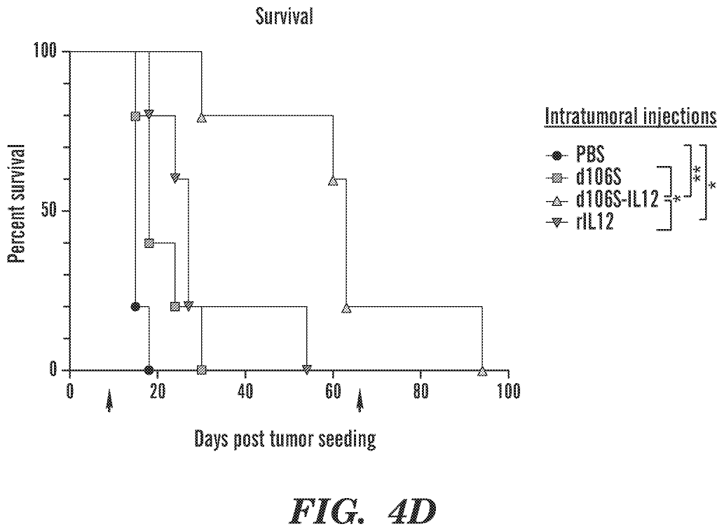

The apparent full tumor regression of one mouse treated with d106S-IL12 in the previous experiment and the expansion of antigen-specific CD8 cells in the other mice (FIG. 3D) suggested a survival benefit with virus. However, given the high toxicity of IL-12^{13,14}, it could be possible that levels of IL-12 secreted by the virus reach dose-limiting toxicities. To determine if the virus promoted survival against melanoma challenge and prevented IL-12 toxicity, we established B16^{Nectin1} tumors in mice and injected mice intratumorally every three days starting on day 7 and measured tumor volume beginning on day 6 after randomization (FIG. 4A). In this experiment, by day 19, all saline-treated mice succumbed to their tumors, while tumors in some of the mice treated with d106S-IL12 were beginning to regress (FIG. 4B). Although d106S treated mice did have a slight reduction in total tumor volume, relative to the starting size at day 6, they grew like controls (FIG. 4C). Recombinant IL-12 did reduce tumor growth (FIG. 4B, 4C) and prolong survival from melanoma (FIG. 4D). However, d106S-IL12 treatment was the most successful treatment, more than tripling the median survival from 18 days with saline to 63 days.

Checkpoint Blockade Does Not Enhance d106S-IL12 Survival Benefit

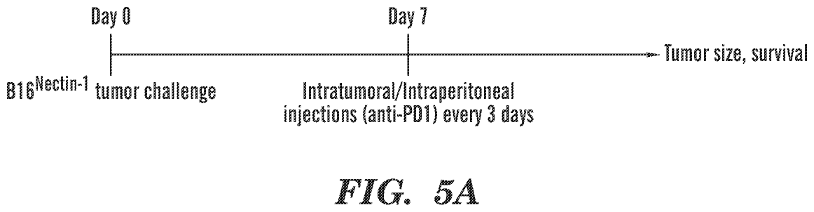

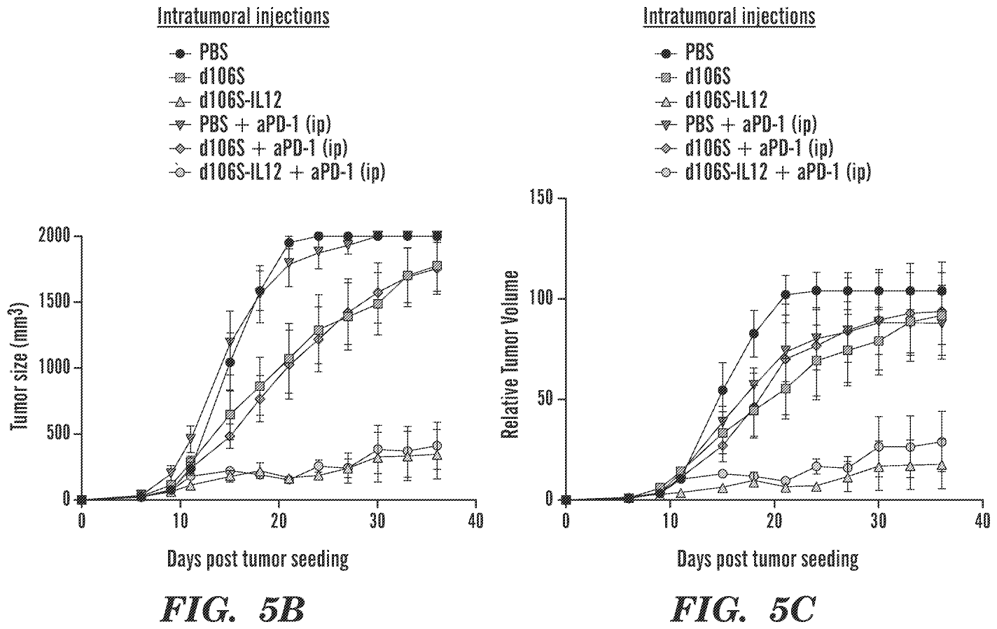

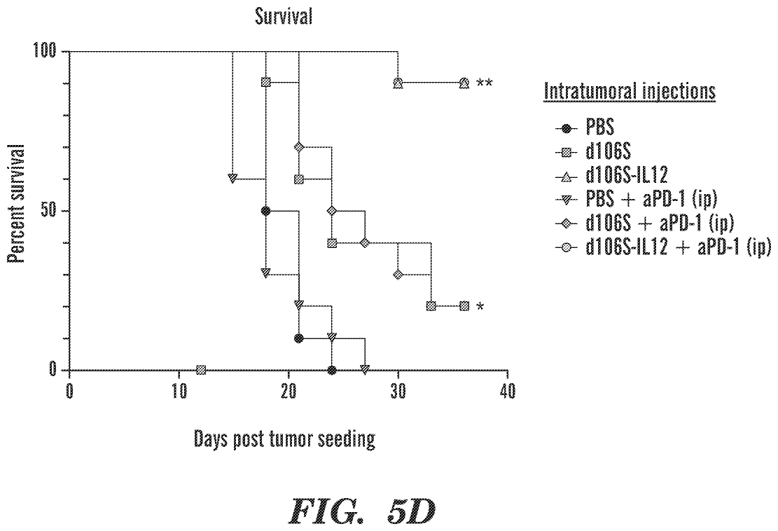

Based on the previous success of immune checkpoint blockade in melanoma¹⁶, and the success of combining anti-PD-1 therapy with the oncolytic virus T-VEC¹⁷, we chose to investigate if we could extend the survival benefit from d106S-IL12 injections with anti-PD-1 checkpoint blockade. Following weeklong growth of B16^{Nectin1} tumors, mice received intratumoral and i.p. injections with anti-PD-1 blocking antibody (100 μg) every three days (FIG. 5A). Mice receiving checkpoint blockade injections and PBS were no more protected than PBS control mice. In fact, comparing between groups there was no difference between those mice that received checkpoint blockade and those that did not: differences were only seen between the various intratumoral treatments. As seen in the previous experiment, most of the mice receiving d106S intratumoral injections were not protected. However, there was a slight impairment of tumor growth and one mouse was even tumor-free with this treatment. Both d106S-IL12 groups benefited from this treatment, with greatly reduced tumor growth and enhanced survival (FIG. 5B, 5C, 5D).

### References

1. Leoni V, Vannini A, Gatta V, . . . Campadelli-Fiume G. A fully-virulent retargeted oncolytic HSV armed with IL-12 elicits local immunity and vaccine therapy towards distant tumors. Hutt-Fletcher L, ed. PLOS Pathog. 2018;14(8):e1007209.

2. Parker J N, Yancey Gillespie G, Love C E, Randall S, Whitley R J, Markert J M. Engineered Herpes Simplex Virus Expressing IL-12 in the Treatment of Experimental Murine Brain Tumors.

3. Toda M, Martuza R, Kojima H, Rabkin S D. In Situ Cancer Vaccination: An IL-12 Defective Vector/Replication-Competent Herpes Simplex Virus Combination Induces Local and Systemic Antitumor Activity. J Immunol. 1998;160.

4. Mizuguchi H, Xu Z, Ishii-Watabe A, Uchida E, Hayakawa T. IRES-Dependent Second Gene Expression Is Significantly Lower Than Cap-Dependent First Gene Expression in a Bicistronic Vector. Mol Ther. 2000;1(4):376-382.

5. Klinke D J. The ratio of P40 monomer to dimer is an important determinant of IL-12 bioactivity. J Theor Biol. 2006;240(2):323-335.

6. Gillessen S, Carvajal D, Ling P, . . . Gately M K. Mouse interleukin-12 (IL-12) p40 homodimer: a potent IL-12 antagonist. Eur J Immunol. 1995;25(1):200-206.

7. Ling P, Gately M K, Gubler U, Hakimi J. Human IL-12 p40 homodimer binds to the IL-12 receptor but does not mediate biologic activity. J Immunol. 1995;154(1):116-127.

8. Lieschke G, Rao P, Gately M, Mulligan R. Bioactive murine and human interleukin-12 fusion proteins which retain antitumor activity in vivo. Nat Biotechnol. 1997.

9. Liu X, Broberg E, Watanabe D, Dudek T, Deluca N, Knipe D M. Genetic engineering of a modified herpes simplex virus 1 vaccine vector. Vaccine. 2009; 27:2760-2767.

10. Orzalli M H, DeLuca N A, Knipe D M. Nuclear IFI16 induction of IRF-3 signaling during herpesviral infection and degradation of IFI16 by the viral ICP0 protein. Proc Natl Acad Sci USA. 2012;109(44):E3008.

11. Eidson K M, Hobbs W E, Manning B J, Carlson P, DeLuca N A. Expression of herpes simplex virus ICP0 inhibits the induction of interferon-stimulated genes by viral infection. J Virol. 2002;76(5):2180-2191.

12. Thomann S, Jan B, Vogel K, . . . Schuster P. Combined cytotoxic activity of an infectious , but non-replicative herpes simplex virus type 1 and plasmacytoid dendritic cells against tumour cells. Immunology. 2015:327-338.

13. Sacco S, Heremans H, Echtenacher B, Ghezzi P. Protective effect of a single interleukin-12 (IL-12) predose against the toxicity of subsequent chronic IL-12 in mice: role of cytokines and glucocorticoids. Blood. 1997.

14. Brunda M J, Luistro L, Warner R R, . . . Gately M K. Antitumor and Antimetastatic Activity of Interleukin 12 against Murine Tumors. J Exp Med. 1993.

15. Dougan S K, Dougan M, Kim J, Ploegh H L. Transnuclear TRP1-specific CD8 T cells with high or low affinity TCRs show equivalent antitumor activity. Cancer Immunol Res. 2013; 1(2):99-111.

16. Wolchok J D, Kluger H, Callahan M K, . . . Sznol M. Nivolumab plus ipilimumab in advanced melanoma. N Engl J Med. 2013;369(2):122-133.

17. Ribas A, Dummer R, Puzanov I, . . . Long G V. Oncolytic Virotherapy Promotes Intratumoral T Cell Infiltration and Improves Anti-PD-1 Immunotherapy. Cell. 2017;170(6):1109-1119.e10.

### Example 2

### Cellular and Molecular Mechanisms of Cancer Equilibrium Induced by d106S-IL12

Tumors in immune equilibrium are held in a balance between outgrowth and destruction by the immune system. The inventors have previously demonstrated the d106S-IL12 can induce an immune equilibrium state in B16 murine melanoma (data not shown). They also discovered that the pro-inflammatory cytokines, TNFα, IL-1β, and IL-6, were tumor promoting in this stage and that depletion of each of these overcame equilibrium in some mice (data not shown). Previous reports have demonstrated the importance of CD4⁺ T cells and CD8⁺ T cells, and IFNγ in maintaining tumors in equilibrium. Additionally, it has been shown that tumors can escape immune pressure by intrinsic mutations to antigen presentation and IFN-sensing pathways. It is shown herein that equilibrium can also be induced in a murine pancreatic cancer tumor, and confirm that IFNγ secretion is essential for maintaining tumors in equilibrium. However, neither CD4⁺ nor CD8⁺ T cells were fully required on their own to maintain tumors in equilibrium. Furthermore, tumors could be controlled in mice that lacked cross-presenting dendritic cells, which are typically thought of as essential for anti-tumor immunity. Direct cytolytic function of CD8⁺T cells and NK cells or the presence of γδ cells were also unnecessary to maintain tumors in equilibrium. Tumors that lacked antigen presentation to CD8⁺ T cells could still be partially controlled by d106S-IL12 and interestingly, tumors lacking both type I and II IFN sensing were kept in equilibrium. These results indicate that d106S-IL12 induced equilibrium control comes from IFNγ, but not necessarily through direct CD8⁺ T cell killing or IFNγ-mediated cytotoxicity.

### Introduction

The immunosurveillance of cancer consists of three phases: elimination, equilibrium, and escape. During equilibrium, tumors are maintained in a state of immune-mediated dormancy, making detection and understanding of this phase a significant challenge (1). Mouse models using the carcinogen methylcholanthrene (MCA) have established tumors in equilibrium and have significantly improved the understanding of this phase of immunoediting, although the models are somewhat cumbersome as tumors are not reliably in equilibrium until up to 200 days after treatment (2,3). In this model, by depleting both CD4⁺ and CD8⁺T cells, interferon-γ (IFNγ), or interleukin-12 (IL-12), tumors maintained in equilibrium escaped from the apparent lack of cellular adaptive immune control (2,3). The anti-tumor effects of IFNγ are well-known as IFNγ binding to tumor cells upregulates major histocompatibility complex class I (MHC-I) to increase antigen presentation, as well as causing cytostatic growth defects in tumors (4-6). Tumors can avoid these negative pressures exerted through IFNγ by mutating genes involved in IFNγ sensing and/or antigen presentation genes, and thus avoid immunosurveillance. Genes that tumors often mutate to become intrinsically resistant to immunotherapies include JAK1/2 and/or STAT1, which are signaling molecules downstream of IFNγ binding, or β2-microglubulin (β2m), which stabilizes MHC-I molecules (7-9). As IL-12 induces expression of IFNγ (10), it follows that depletion of IL-12 hinders IFNγ production and allows for tumor escape from equilibrium.

The inventors previously confirmed the importance of IL-12 in maintaining equilibrium as mice bearing murine melanoma tumors that stopped receiving d106S-IL12 therapy rapidly succumbed to their tumors. Here, the essential role that IFNγ plays in maintaining tumors in equilibrium is confirmed. However, it was discovered that CD4⁺ and CD8⁺ T cells played mostly redundant roles in regulating immune equilibrium as depletion of either of these cell types alone did not significantly increase tumor escape. While conventional T cells played a significant role in their maintaining stable tumors, other cell types also contributed to slowing tumor growth. However, mice lacking γδ T cells, cross-presenting dendritic cells (DCs), or mice lacking direct cytolytic activity of CD8⁺ T cells and NK cells had stable tumors when treated with d106S-IL12, suggesting that these cell types and functions were not necessary for establishing equilibrium.

The model described herein rapidly establishes equilibrium in under 40 days, and also allows for genetic manipulation of tumors, which is not possible with a carcinogen-based equilibrium model. The genetic manipulation of tumors allows for introduction of mutations that might generate tumor intrinsic resistance to immune pressure and subsequent escape from equilibrium. As such, CRISPR-Cas9 knockouts of β2m, STAT1, and IFNAR were generated to test the role of antigen presentation and IFN sensing during d106S-IL12 therapy. Surprisingly, although the essential role that IFNγ plays in maintaining tumors in equilibrium were validated, STAT1^{-/-} tumors were controlled by d106S-IL12 treatment and β2m^{-/-} tumors were partially controlled. This indicated that tumor extrinsic effects caused by IFNγ, and not its direct effect on the tumors through cytostatic activity or enhanced antigen presentation, were responsible for immune mediated control. These findings have important implications for tumors that commonly resist immunotherapies through mutations in IFNγ sensing or antigen presentation pathways.

### Results

Murine Pancreatic Cancer is Susceptible to d106S-IL12 Induced Equilibrium that is Independent of Direct CD8⁺ T Cell Recognition

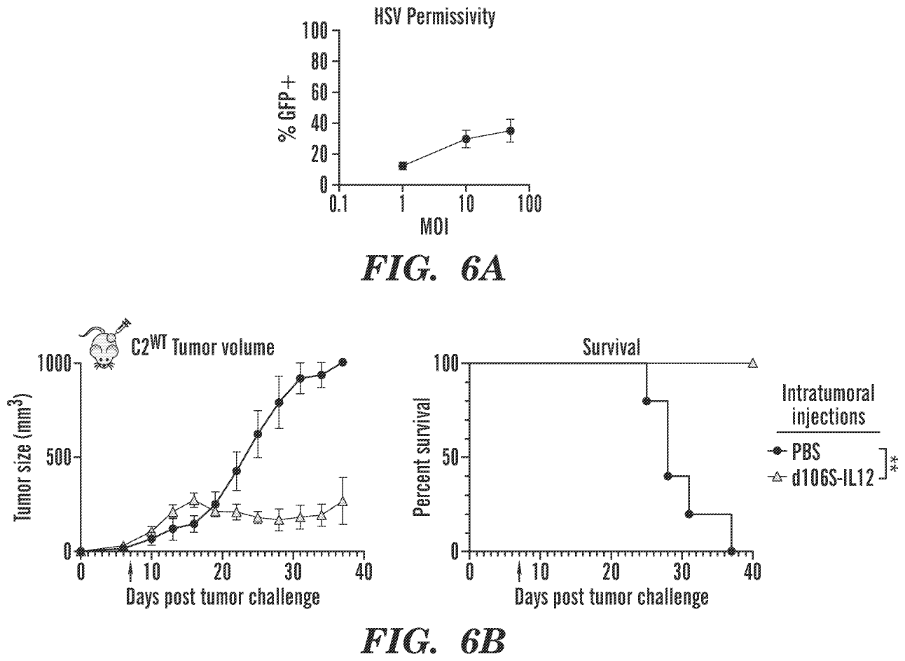

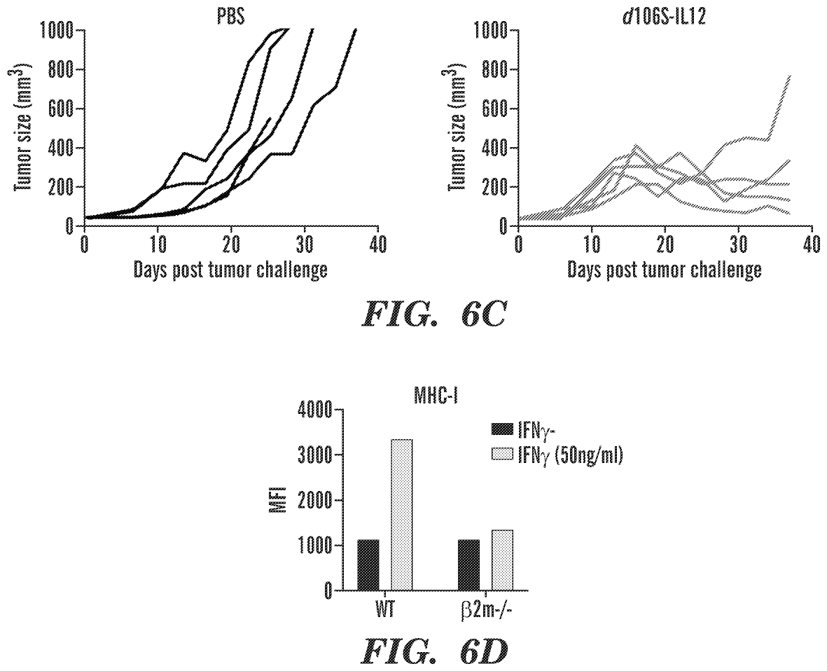

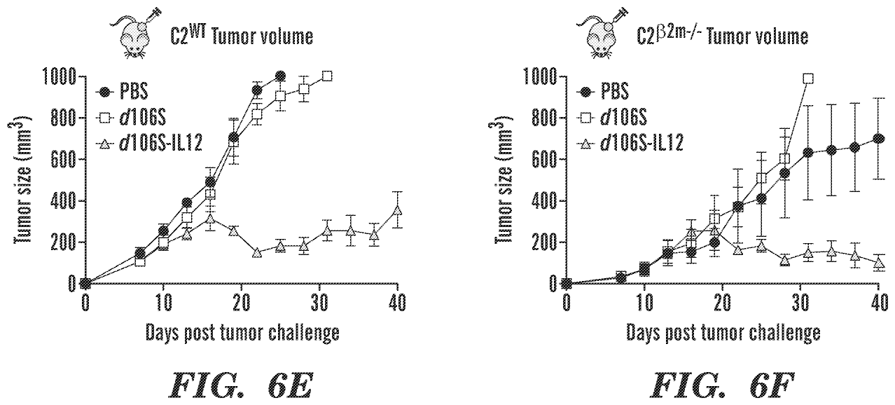

The inventors previously demonstrated that d106S-IL12 treatment can induce stable tumors in the B16 mouse model of melanoma, rendered permissive to HSV infection through nectin1-transduction (B16^{Nectin1}) It was first sought to determine if this model of equilibrium establishment was possible in more than just melanoma. To test if this, use of the poorly immunogenic, T cell-low pancreatic cancer clone 6694c2 (11), herein referred to as C2 was chosen. Unlike wild-type B16 cells, which are completely non-permissive to HSV infection (data not shown), C2 cells had naturally moderate levels of HSV infection (FIG. 6A), albeit at a much lower level compared to B16^{Nectin1} cells (data not shown). Upon subcutaneous tumor inoculation in the flanks of mice, d106S-IL12 treatment was begun at day seven and continued every three days. Compared to PBS injection, C2 tumors injected with d106S-IL12 significantly slowed tumor growth and extended survival of mice (FIG. 6B). Indeed, continuous treatment of subcutaneous pancreatic cancer tumors with d106S-IL12 led to stable disease in most mice compared to PBS treated animals (FIG. 6C).

As these tumors readily established equilibrium with d106S-IL12 treatment, it was determined if genetic ablation of antigen presentation impaired the immune response to virotherapy. β32-microglobulin (β2m) stabilizes the presentation of endogenous peptides on MHC-I and as such, mutation to this gene is a common mechanism to avoid immune surveillance through T cell receptors (TCRs) on CD8⁺ T cells (6,12). β2m knockout C2 tumor cells were generated by CRISPR-Cas9 mutagenesis. As MHC-I is readily induced by IFNγ (4), the knockout of β2m was functionally confirmed through the lack of MHC-I upregulation on the surface of β2m^{-/-} cells compared to wild-type cells (FIG. 6D). C2^{WT} and C2^{β2m-/-} were established in mice and treated with PBS, d106S, or d106S-IL12. Unlike previous results in for B16 melanoma, d106S alone did not provide any additional benefit over PBS in this pancreatic cancer model (FIG. 6E-6F), indicating that type I IFN may not play a role in controlling these tumors. As seen before, d106S-IL12 treatment of C2^{WT} tumors generated stable tumors (FIG. 6E). Surprisingly, β2m^{31/-} tumors responded very well and like the wild-type tumors, they entered a stable immune equilibrium (FIG. 6F), indicating that for this model, immune control could be mediated without direct CD8⁺ T cell recognition of tumor cells.

Equilibrium is Maintained by IFNγ, while CD8⁺ and CD4⁺ T Cells Provide Redundant Protection

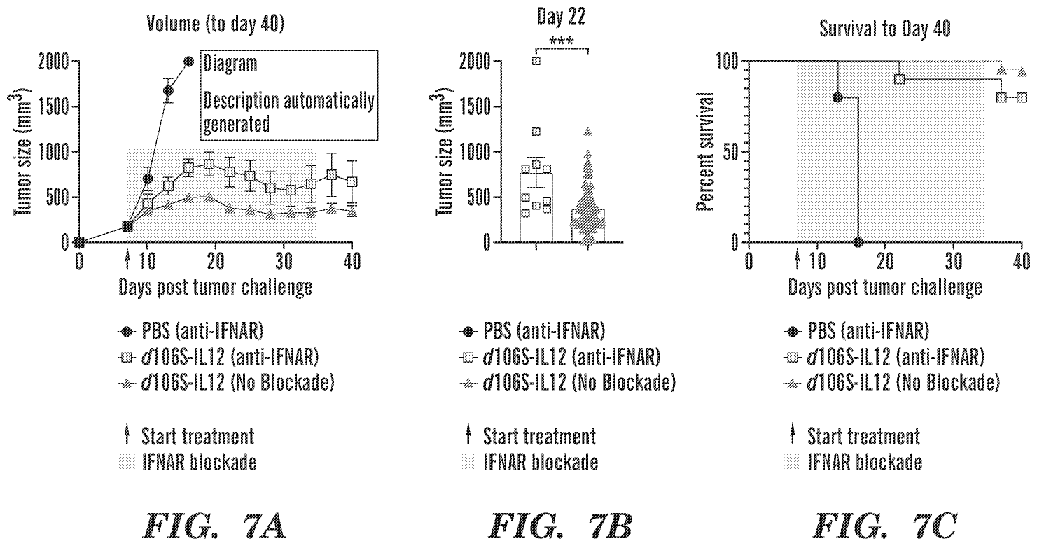

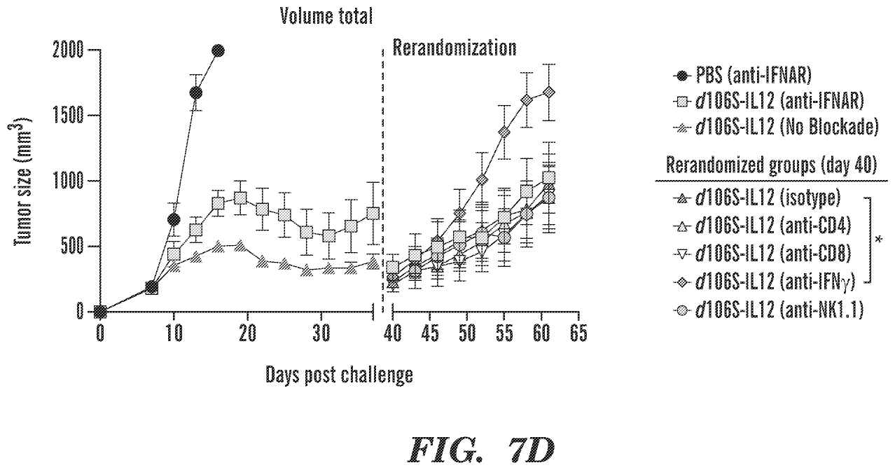

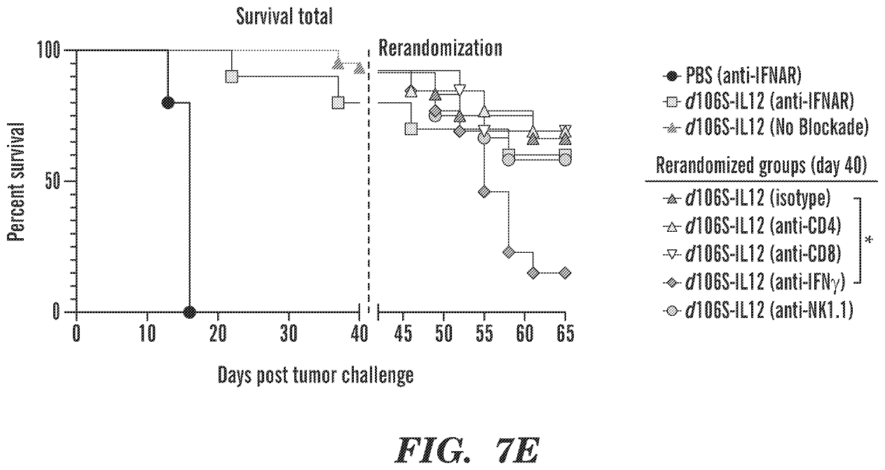

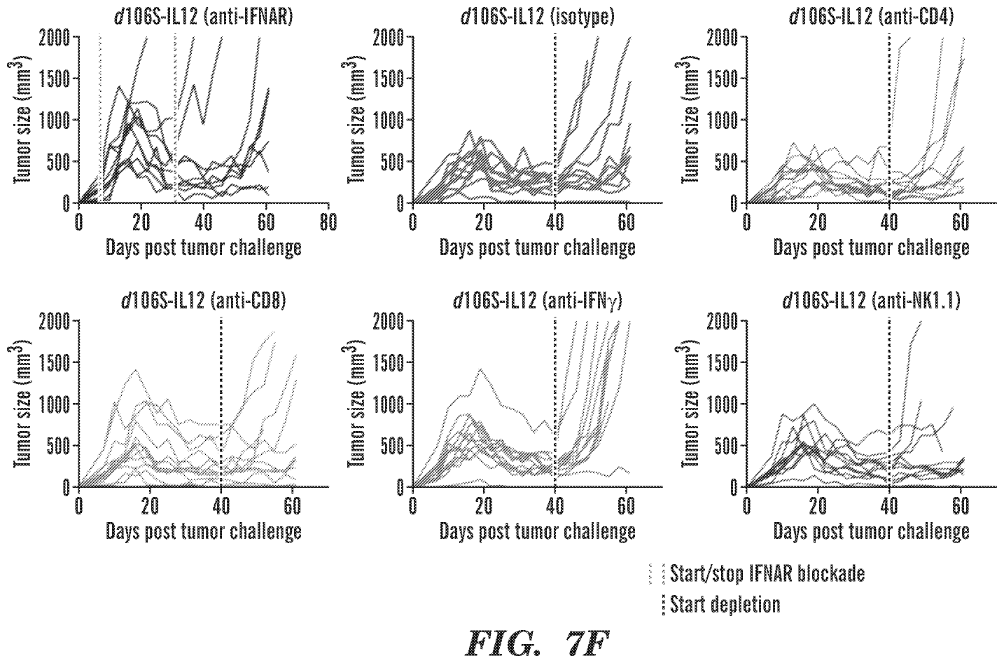

As previously mentioned, the lack of a response to d106S in the C2 model, suggests a limited role for type I IFN in tumor control. It was previously in the B16 model that d106S could induce a type I IFN response (data not shown), thus the inventors returned to experiments using B16 as the bulk of the understanding of the immune equilibrium was in this model. To test the role of type I IFN in the generation of immune equilibrium and response to d106S-IL12, B16^{Nectin1} (hereafter referred to as B16) tumors were established in mice and began treating with PBS or d106S-IL12 starting at day seven and continued treatment every three days. On the first day of intratumoral therapy, injections also began with an anti-IFNAR (type I IFN receptor) blocking antibody. As seen in previously, d106S-IL12 readily induced stable disease of B16 tumors (FIG. 7A). Interestingly, tumors treated with IFNAR blockade grew slightly faster than those that had normal type I IFN signaling (FIG. 7A). However, around day 20-22 of tumor growth, the anti-IFNAR treated mice had stabilized their tumors (FIG. 7A). At this peak of tumor growth the mice receiving IFNAR blockade had significantly larger tumors, but their tumors were still held in equilibrium (FIG. 7B), leading to extended survival (FIG. 7C). This indicates that although there was an initial benefit of type I interferon from the virus itself, at later time points adaptive immunity initiated from the release of IL-12 was highly protective.

In addition to type I IFN, the role of other immune mediators in maintaining equilibrium was also tested. equilibrium in B16 tumors in a large cohort of mice using d106S-IL12 was established. At day 40, mice were rerandomized from this large group into new groups to receive either anti-CD4, anti-CD8, anti-IFNγ, anti-NK1.1, or isotype control depleting antibodies. These mice also continued to receive d106S-IL12 treatment. Interestingly, it was found that IFNγ blockade was the only depletion that significantly altered tumor growth and overall survival (FIG. 7D-7F). Though there was some stochastic outgrowth of isotype treated tumors, there was no difference in tumor sizes or survival of these mice compared to either CD4 or CD8 depletions, or NK cell depletions (FIG. 7D-7F).

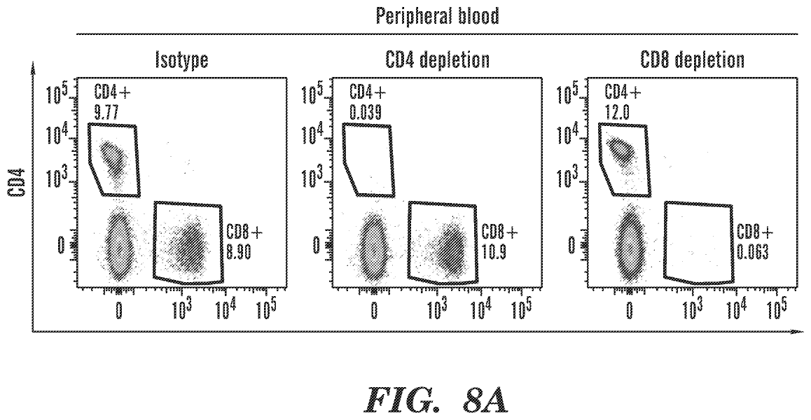

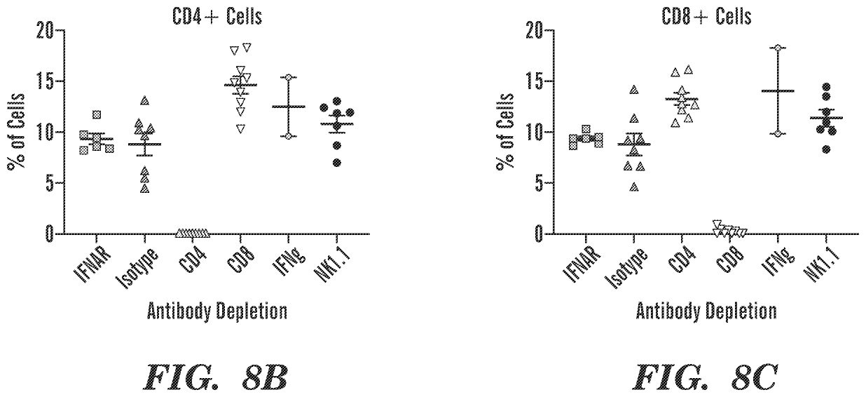

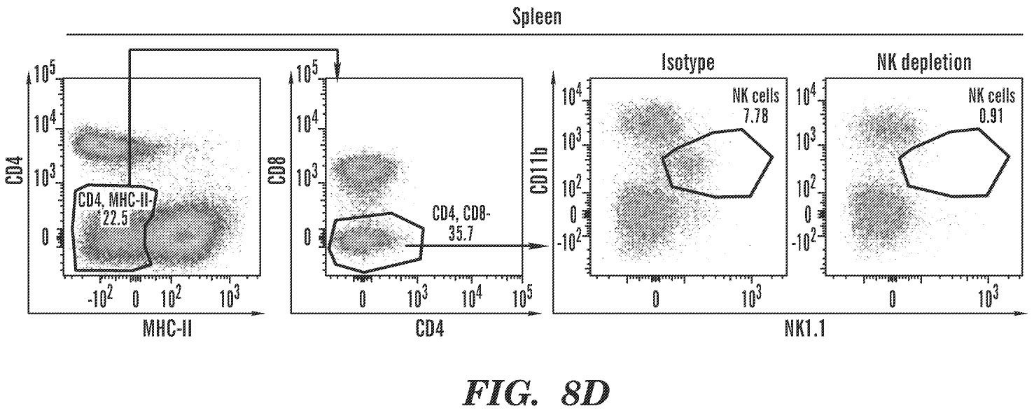

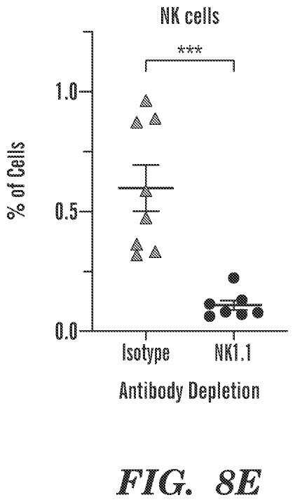

To ensure that antibody depletions worked as expected, peripheral blood and spleens were collected following sacrifice of the surviving mice at day 63. By flow cytometry, it was discovered that CD4⁺ and CD8⁺ T cell populations were almost completely absent in the respective depletion groups (FIG. 8A-8C). Similarly, NK cells were absent in mice treated with anti-NK1.1 depleting antibodies (FIG. 8D-8E). These data indicate that these cell populations were missing from mice that were controlling their tumors, implying that, unlike IFNγ, these cellular factors (i.e. NK cells, CD8⁺, or CD4⁺ T cells) alone were not necessary for maintaining equilibrium.

αβ T Cells Play an Important, But Not Comprehensive, Role in d106S-IL12 Mediated Tumor Control

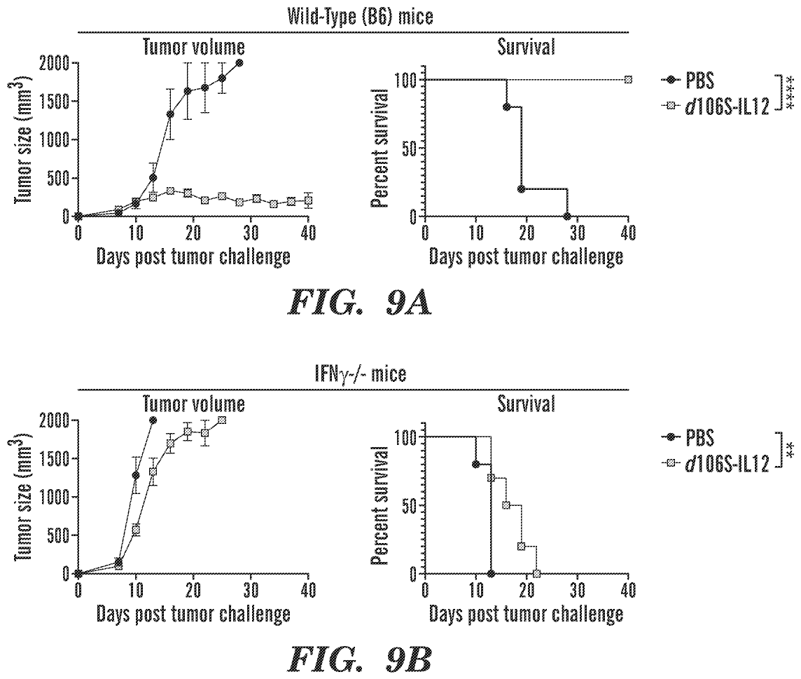

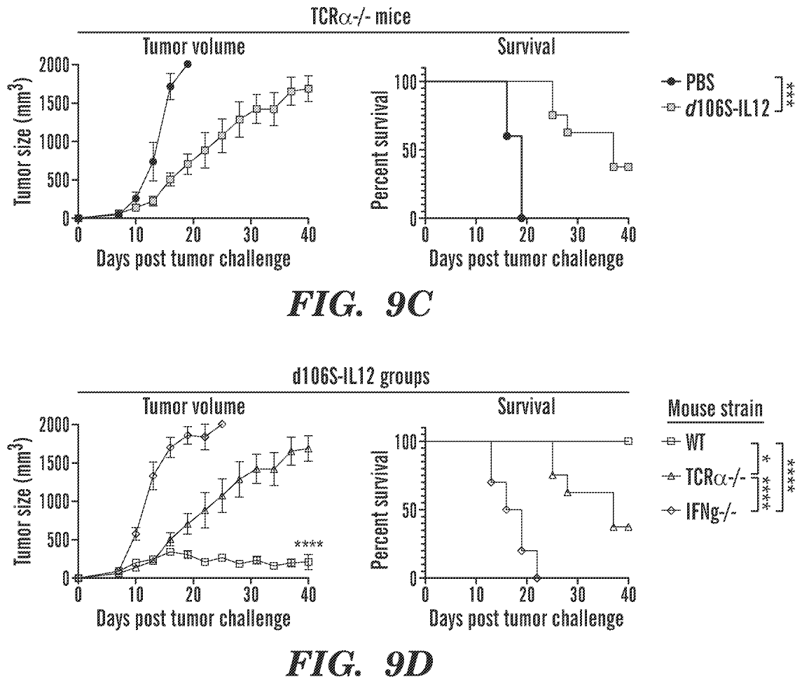

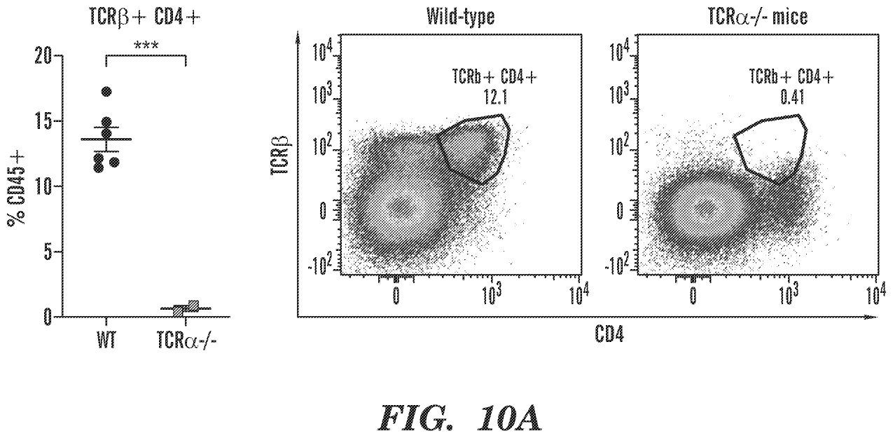

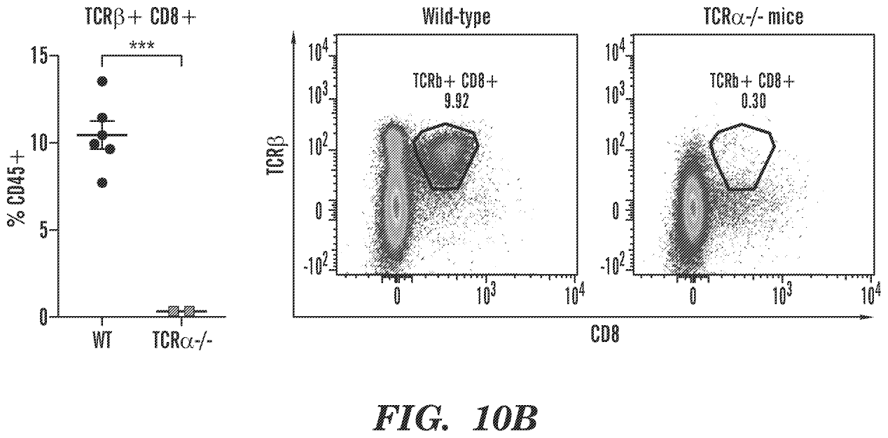

Although it was confirmed that IFNγ was necessary while CD4⁺ and CD8⁺ T cells likely played overlapping roles in equilibrium maintenance, the role of these factors in establishment of equilibrium was tested. To this end, either wild-type, IFNγ^{-/-}, or TCRα^{-/-} mice were inoculated with B16 tumors and began treatment with either PBS or d106S-IL12 on day seven and onwards every three days. As seen before, wild-type mice treated with d106S-IL12 had stable tumors (FIG. 9A). IFNγ^{-/-} mice were unable to control their tumors during d106S-IL12 treatment, further demonstrating that IFNγ is critical for establishing equilibrium (FIG. 9B). Intriguingly, TCRα^{-/-} mice did not stabilize their tumor growth with d106S-IL12 treatment like wild-type mice, but tumor growth was slowed significantly over PBS treatment (FIG. 9C). It was again confirmed these results were due to absence of T cells through flow cytometry: both CD4⁺ and CD8⁺ T cells (TCRβ⁺) were appropriately absent in these mice (FIG. 10A and 10B). This intermediate phenotype (FIG. 9D) indicated that αβ T cells are critical for stabilizing tumor growth and establishing equilibrium, but they are not the only cell types capable of slowing tumor growth during d106S-IL12 treatment.

Establishment of Equilibrium from d106S-IL12 Treatment Does Not Rely on γδ T Cells, Cross Presenting DCs or Perforin/Granzyme

It was confirmed that IFNγ was necessary, but CD4⁺ and CD8⁺ T cells played seemingly overlapping roles in maintaining stable tumors during d106S-IL12 treatment (FIG. 7A-7E). Based on these results, it was originally hypothesized that through their secretion of IFNγ, tumor growth could be controlled by either CD4⁺ or CD8⁺ T cells. However, as TCRα^{-/-} mice grew tumors more slowly than IFNγ^{-/-} mice (FIG. 9D), it was hypothesized that another cell type could be secreting enough IFNγ to maintain tumor control. While TCRα^{-/-} lack αβ T cells, which make up the majority of T cells and include CD4⁺ and CD8⁺ T cells, they still have γδ T cells. Although γδ T cells comprise a small fraction of the total T cell number, they play an important role in an early anti-tumor response (13). It has been shown that mice lacking γδ T cells are less protected from injected tumors growing out compared to mice lacking αβ T cells. These mice lacking γδ T cells were similar to IFNγ^{-/-} mice in their inability to control tumor challenge. Indeed, tumor control by γδ T cells seems to rely heavily on IFNγ as mice that have IFNγ-deficient γδ T cells have decreased protection from injected tumor challenge (13).

Another rarer population of cells that often play critical roles in an anti-tumor response are cross-presenting DCs. These DCs, which a require the transcription factor BATF3 for development, can efficiently phagocytose and present antigens to CD8⁺ T cells to prime an anti-tumor response (14). Though cross-presenting DCs are best known for this ability, they are also capable of recruiting T cells through their chemokine secretion, as well as also being another possible source of IFNγ (15,16). Therefore, even though CD8⁺ T cells were depleted, cross-presenting DCs could still play a part in an anti-tumor response.

Finally, though CD8⁺ T cells and NK cells were depleted separately, it is possible that there was a coordination between these cytotoxic cells and that a co-depletion would have caused tumors to escape from equilibrium. Both CD8⁺ T cells and NK cells lyse target cells primarily through degranulation of cytotoxic vesicles containing perform and granzymes. Perform generates pores in the target cell's plasma membrane so that granzymes can enter to activate apoptosis (17).

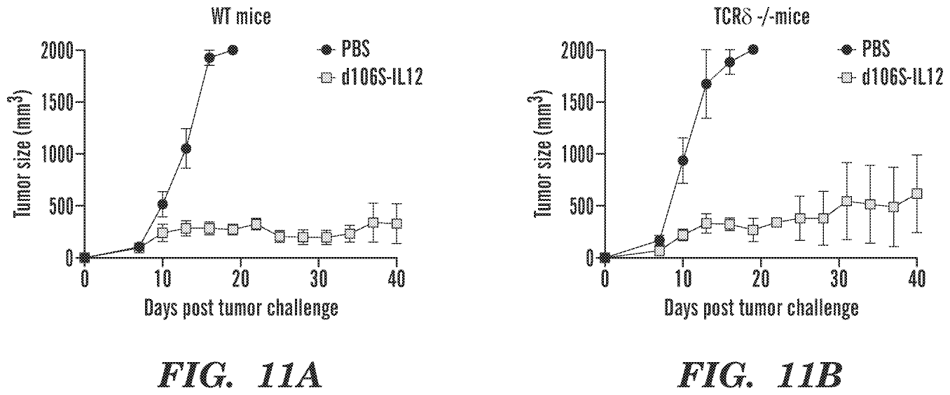

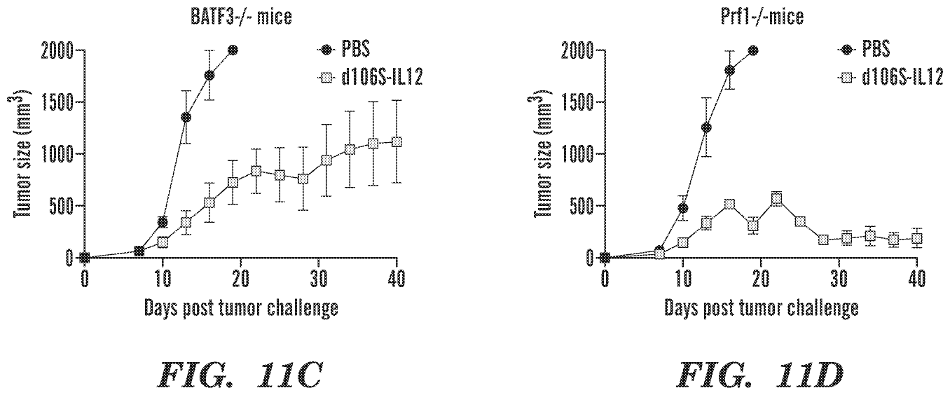

Consequently, the role of γδ T cells, cross-presenting DCs, and direct cytolytic activity of NK cells and CD8⁺ T cells during d106S-IL12 treatment were tested by inoculating either wild-type TCR^{-/-}, BATF3^{-/-}, or perforin^{-/-} (Prfl^{-/-}) mice, respectively with B16 tumors. Surprisingly, all the mice treated with d106S-IL12 had reduced tumor burdens compared to PBS treated mice (FIG. 11A-11D). Although some knockout mice, such as BATF3^{-/-} grew tumors that were slightly larger than their wild-type counterparts, most of the mice stabilized their tumor growth and entered into immune equilibrium (FIG. 11A-11D). These results indicated that none of these cell types, nor the direct cytotoxic function of CD8⁺ T cells and NK cells were necessary on their own for establishment of equilibrium.

Tumor Sensing of IFNγ is Unimportant for d106S-IL12 Induced Equilibrium

In the setting of C2 pancreatic cancer, the absence of a direct CD8⁺ T cell response against β2m^{-/-} tumors still led to establishment of equilibrium (FIG. 6F). Although the CD8 depletion during B16 equilibrium also suggested that lacking CD8⁺ T cells did not affect equilibrium maintenance (FIG. 7D-7F), it was sought to confirm this using knockout tumor cell lines. In addition to mutations in the antigen presentation pathway, such as (32m, tumors can have loss of function mutations to the IFN sensing pathway to avoid immunosurveillance. STAT1 is downstream of both type I (IFNα/β) and type II (IFNγ) signaling, and as such STAT1^{-/-}/tumors become unresponsive to many immunotherapies (4,6,18). Therefore, B16 cells that were β2m or STAT1 deficient were generated using CRISPR-Cas9 to test their response to d106S-IL12. An early effect of type I IFN through IFNAR blockade (FIG. 7A-7B) was seen, and thus, the role of type I IFN sensing was tested by generating IFNAR knockout B16 cells.

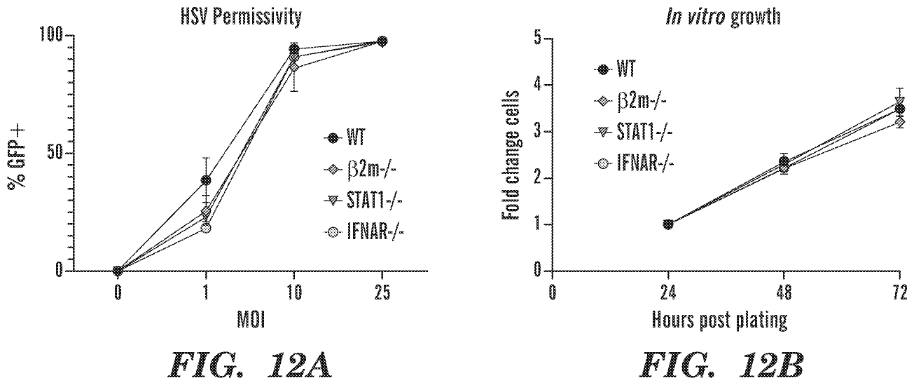

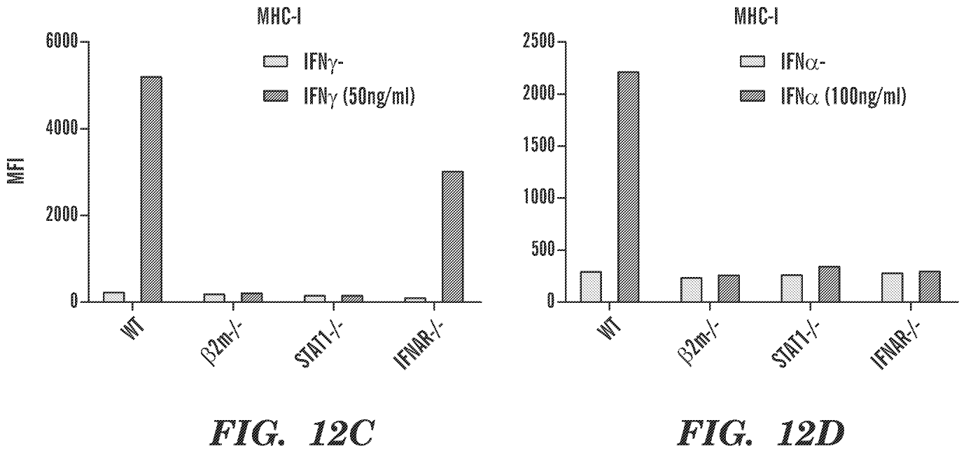

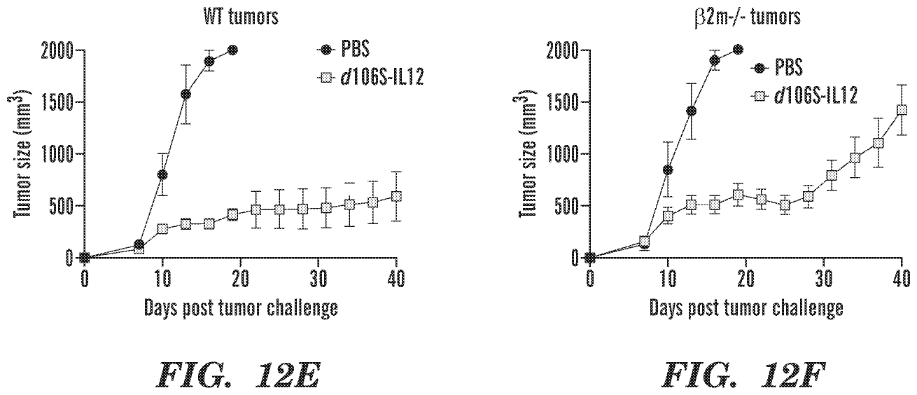

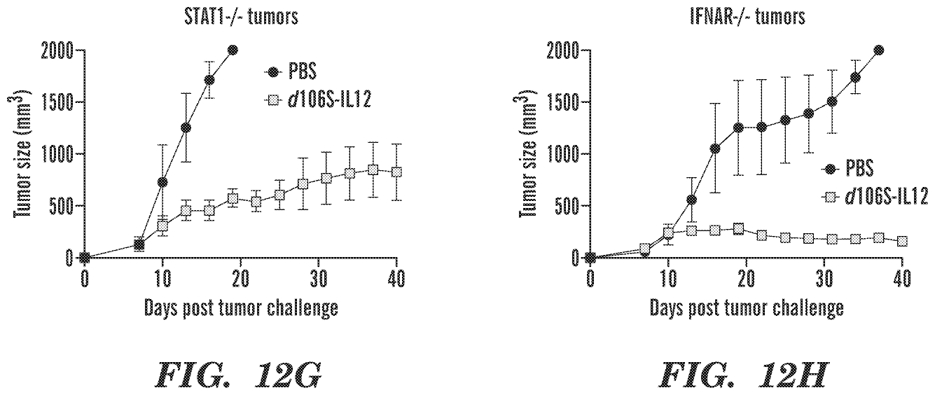

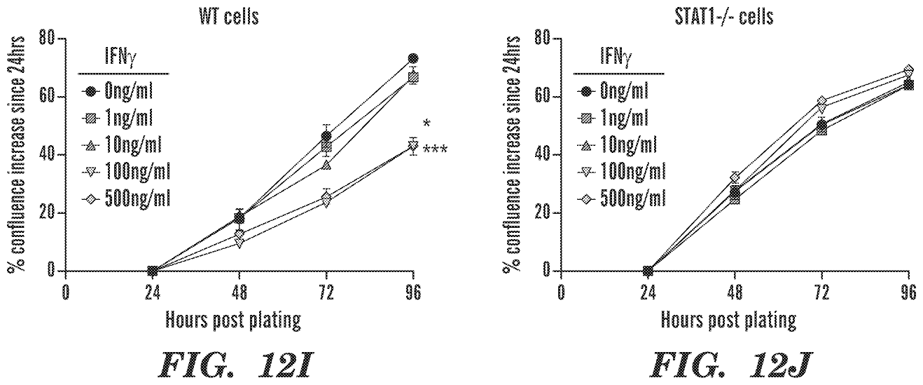

Generation of B16 knockouts was done in a B16^{WT;Nectin1-} background, so the knockout cells were transduced with a lentivirus encoding nectin-1 (19) to allow for efficient d106S infection (20). Knockout cells that could be infected to similar levels as the B16^{WT;Nectin1+} were selected (FIG. 12A), and cells that grew in vitro at a similar rate (FIG. 12B). It was confirmed that nectin1+ transduction had no effect on IFNAR, β2m, or STAT1 deletions by monitoring MHC-I upregulation following the addition of IFNγ or IFNα. As expected, β2m^{-/-} cells did not express MHC-I, STAT1^{-/-} cells did not upregulate MHC-I in response to either type of IFN, and IFNAR^{-/-} cells only responded to IFNγ (FIG. 12C).

Following in vitro verification of cell lines, these cells were injected into separate groups of wild-type mice to form tumors. All knockout tumors responded, albeit to varying degrees, to d106S-IL12 injection compared to PBS control (FIG. 12E-12H). Interestingly, β2m^{-/-} tumors responded initially, but many began to grow out, suggesting a partial response. However, almost all the β2m^{-/-} tumors that did grow out began at larger sizes than their wild-type counterparts (FIG. 12E-12F), which suggests perhaps they grew better in vivo than their in vitro growth indicated (FIG. 12B). Unlike the IFNAR antibody blockade experiment (FIG. 7A-7B), IFNAR^{-/-} tumors responded to the same degree as wild-type (FIG. 12H), which suggested that type I IFN was involved in coordinating an anti-tumor response early but did not necessarily act directly upon the tumors to slow their growth.

Most surprisingly, STAT1^{-/-60} tumors continued to respond to d106S-IL12 treatment and entered immune equilibrium despite lacking a response to IFN (FIG. 12G). Because it was confirmed how important IFNγ was for immunosurveillance when depleted with antibodies (FIG. 7D-7F) or used genetic knockout mice (FIG. 9B), it was hypothesized that IFNγ was likely acting directly on tumors to slow their growth, and/or increase antigen presentation. Indeed, it was confirmed that growth of wild-type B16 cells in vitro could be slowed with high concentrations of IFNγ (FIG. 12I). However, as the growth of STAT1^{-/-} cells was unaffected by the presence of IFNγ (FIG. 12J), this cytostatic activity could not account for the mechanism by which IFNγ was necessary for establishment and maintenance of immune equilibrium. This suggested that the main effect of IFNγ was not acting directly on the tumor, but instead coordinating an immune response through a variety of possibly redundant cell types to control and stabilize tumor growth.

### Discussion

CD8⁺ T cells are one of the key players in mediating tumor cell destruction, as their presence is prognostically favorable for disease outcome during immunotherapy (11,21). Here, it is shown that CD8⁺ T cells play a partially redundant role, and their direct recognition and lysis of tumor cells is not necessarily required for slowing of tumor growth. Previous equilibrium studies have depleted both CD8⁺ and CD4⁺ T cells concurrently, which resulted in tumor outgrowth (2,3). Through individual depletions of CD8⁺ or CD4⁺ T cells, a concomitant increase was observed in the corresponding other T cell type, which has been seen before with depletions (22), and may partly explain how tumors were controlled. BATF3^{-/-} mice grew tumors similar to how WT mice grew B16^{β2m-/-} tumors, that is, significant tumor control, but not as complete as WT mice growing WT tumors. This suggests that CD8⁺ T cells do still play a small role in controlling later stage tumors during d106S-IL12 treatment. Although both conventional T cell types were not depleted in experiments presented herein, TCRα^{-/-} mice demonstrate that αβ T cells play an important role in establishing immune equilibrium, although these cell types alone could not fully explain the partial response observed in the knockout animals. However, other cell types on their own, including NK cells, γδ T cells, and BATF3^{-/-} cross-presenting DCs were also not required to slow tumor growth and establish or maintain equilibrium. It is likely that the immune response generated by d106S-IL12 involves many overlapping features of the immune system, and no one cell type can mediate the entire effect.

For example, in the absence of CD8⁺ T cells, tumors could be controlled by NK cells. Using STING agonists for activation, NK cells can target β2m^{-/-} tumors that would normally evade CD8⁺ T cell killing (23). This effect is highly dependent on type I interferons acting directly on NK cells (23). Virus described herein induce a type I interferon response (data not shown). Indeed, both type I IFN and IL-12 can enhance NK cell cytotoxicity (25). As NK cells sense the absence of MHC-I on the surface of cells (17,24), enhanced NK cell targeting of β2m^{-/-} tumors might explain the protection d106S-IL12 provided against C2^{β2m-/-} tumors. However, β2m^{-/-} B16 tumors are less sensitive to NK cell killing compared to other tumor types (23), which might explain the partial response in B16^{β2m-/-} tumors observed.

Although NK cells and CD8⁺ T cells are thought of as the main cytotoxic cell types, CD4⁺ T cells have also been observed to directly lyse cells (26). The inventors previously demonstrated that d106S could induce upregulation of MHC-II and both human and melanoma tumors are known to express MHC-II (27,28). Therefore, it is reasonable that CD4⁺ T cells mediate tumor control through direct cytotoxicity from expression of perforin and granzymes (26). However, it was also demonstrated that Prfl^{-/-} animals were fully capable of tumor control, which suggests that direct cytotoxicity through perforin/granzymes through either NK cells or CD8⁺ or CD4⁺ T cells was not necessary for equilibrium establishment and maintenance in model described herein. This is somewhat surprising as depletion of various components of innate immunity, including perforin, often leaves animals more prone to spontaneous tumor formation (1,29). However, as the B16 tumors are implanted and not spontaneous, it is likely that once a tumor has taken hold in mice, perforin plays a less important role. Conversely, tumors engineered to express IL-12 have been shown to no longer be cleared by the immune system in mice lacking perforin (30), suggesting again that d106S-IL12 induces overlapping effects on the immune system with compensatory mechanisms for control.

This redundant role of multiple cell types was also made clear in both the TCRδ^{-/-} and BATF3^{-/-} mouse experiments. The inventors previously found that RAG2^{-/-} animals, which lack all B and T cells, could barely control tumors. However, the inventors now show that TCRα^{-/-} animals had an intermediate phenotype in response to d106S-IL12, suggesting that either B cells or a non-4 T cell could mediate some protection. Although the inventors did not try implanting tumors in mice lacking B cells (μMT mice), TCRδ^{-/-} mice described herein showed no difference compared to WT mice, which may suggest that while important in absence of αβ T cells, γδ T cells are also redundant players during d106S-IL12 treatment. Although many studies have shown that mice deficient in BATF3 have severely impaired anti-tumor immunity (31-33), partial protection in BATF3^{-/-} mice was seen, which could be attributed to the somewhat redundant role that CD8⁺ T cells play in response to d106S-IL12 treatment. Additionally, it has been shown that BATF3^{-/-} mice can still develop cross-presenting cDC1s in response to IL-12 administration and that these knockout mice can achieve tumor clearance in response to IL-12 (34). Therefore, mice treated with d106S-IL12 may have developed cross-presenting DCs, though this would need to be verified.

Despite the overlapping role of cell types tested, the central player in d106S-IL12 mediated immune control of tumors is IFNγ. Although a slight survival advantage in IFNγ^{-/-} mice treated with d106S-IL12 was observed, this could possibly be attributed to the type I IFN response generated by the virus (data not shown). Clearly type I IFN plays an important role early on in an anti-tumor response, as IFNAR blockade increased tumor size. However, this effect was not due to direct type I IFN cytotoxic effects on the tumor cells (6,35) because IFNAR^{-/-} tumors were easily controlled by d106S-IL12 treatment. Additionally, after the initial immune mediated orchestration for tumor control, type I interferon became unnecessary. Thus, type I IFN must coordinate an immune response initially, though this provides a limited benefit to IFNγ^{-/-} animals.

Numerous studies have demonstrated the importance of IFNγ for immunosurveillance of tumors (2,3,13). This importance is underscored by several genetic screens as well as mutations identified in patients with resistance to immunotherapy converging on alterations in γ sensing by tumors as a general mechanism for immune escape (7,8,36). As such, it was anticipated the importance of IFNγ in the system described herein was through its direct action on the tumor to slow its growth and increase antigen presentation (6). Surprisingly, STAT1^{-/-} tumors, which have no type I or type II IFN sensing, were still well controlled by d106S-IL12. Although wild-type tumor growth was slowed by IFNγ, these results suggest rather than direct effects of IFNγ on the tumor, this cytokine was predominantly needed to coordinate an immune response to control tumor growth.

The main immune cell types that IFNγ can bind to include T cells and NK cells, but also antigen presenting cells (APCs), such as monocytes, macrophages, and dendritic cells (4). IFNγ can enhance the activation of macrophages to express nitric oxide synthase (NOS) to release reactive nitrogen oxide species and increase their phagocytic function (10). Nitric oxide species may damage tumor cells (4), but also can promote immunosuppression (1,37). NOS^{-/-} mice exist and could be used to test if these reactive nitrogen species play a role in controlling or promoting tumor growth during d106S-IL12 treatment. Tumor cell control by macrophages can also be due to enhanced phagocytosis. Recently, it has been shown that T cells can repolarize macrophages, allowing increased tumor killing by macrophages through phagocytosis (33). This effect was independent of direct T cell recognition of tumors and of IFNγ sensing by the tumor (33), which is similar to what was seen. Although IFNγ was less important to repolarize macrophages in that study (33), it is possible that macrophages could be activated by IFNγ downstream of d106S-IL12 in order to control tumor growth.

### Methods

Cell Culture

B16F10 cells were purchased from American Type Culture Collection (ATCC). 6694c2 (C2) pancreatic cancer cells (11) were a gift from Dr. Ben Stanger. CRISPR modifications were performed using pSpCas9(BB)-2A-Puro (PX459) V2.0 vector that was a gift from F. Zhang (Addgene 62988). Single-guide RNAs were cloned into the vector by restriction enzyme digest (BbsI) and ligation with T4 DNA ligase. Cells were transfected with Lipofectamine Stem reagent and used according to the manufacturer's protocol. Cells were selected with puromycin for successful transfection and FACS-sorted for purity, and absence of protein was confirmed by flow following addition of murine IFNγ (50 ng/ml) or IFNα (100 ng/ml) (Peprotech) to the media (33). B16^{WT} and knockout cells were transduced with a previously described lentivirus encoding human nectin-1 (hNectin-1 vector a gift from Dr. Antonio Chiocca (19). Following hygromycin selection (500μg/m1), expression of nectin-1 was validated by infecting B16^{Nectin1} cells with d106S virus and selecting clones that were GFP-positive by flow cytometry analysis (FACSCalibur). The Vero-based E11 complementing cell line (38), which expresses ICP27 and ICP4, was used to grow d106S virus. B16, and E11 cells were cultured in DMEM with 10% heat-inactivated FBS and 1% PenStrep.

Plasmids

The coding sequence for the murine IL-12 fusion protein was cloned from Tandem p40p35, a gift from Nevil Singh; Addgene plasmid #108665. The sequence was amplified with XhoI_IL12_Fow (5′-GCGAGTCTCGAGATGTGTCCTCAGAAGCTAACC-3′ (SEQ ID NO: 29)) and NotI_IL12_Rev (5′-ATAGAAGCGGCCGCTCAGGCGGAGCTCAGATAG-3′ (SEQ ID NO: 30)) primers (IDT). The PCR product was cloned into the pd27B shuttle plasmid by XhoI/NotI (NEB) digestion, followed by Quick Ligation (NEB). The correct insertion into the shuttle plasmid was confirmed by Sanger sequencing (Harvard Biopolymers Facility).

Recombinant Virus Generation

The pd27-IL12 the shuttle plasmid was linearized by SwaI (NEB) digestion. E11 complementing cells were co-transfected with linear pd27B-IL12 and infectious d106S DNA. Infectious d106S DNA was isolated as described previously (39). The progeny viruses were harvested and fluorescence-negative plaques were isolated and purified three times. Each plaque isolate was analyzed for evidence of IL12 insertion and subsequent lack of GFP by PCR. Viral stocks of both d106S and d106S-IL12 were grown and titered on E11 complementing cells (38). IL-12 production was confirmed by IL12p40 ELISA (BioLegend) of infected cell supernatants.

Animals

C57BL/6J, IFNγ^{-/-}, TCRα^{-/-}, TCRδ^{-/-}, BATF3^{-/-}, and Prfl^{-/-} mice were purchased from Jackson Laboratories and housed in the Dana-Farber Cancer Institute Animal Resources Facility. All animal experiments were performed in accordance with the DFCI IACUC-approved protocols.

Tumor Inoculations and In Vivo Experiments

B16^{Nectin1} and C2 cells were screened prior to in vivo use for murine pathogens, including mycobacteria (Charles River Laboratories). B16^{Nectin1} or C2 cells were cultured until 80-90% confluent, trypsinized, washed and resuspended in Hank's balanced salt solution (HBSS) at 2×10⁶ cells/mL or 8×10⁵ cells/mL, respectively. Mice were shaved and 250 μL (5×10⁵ or 2×10⁵) cells were injected subcutaneously in the left flank. For survival experiments, tumor size was measured every three to four days by precision calipers. Mice were euthanized when tumor volume exceeded 2,000 mm³ for B16, 1,000 mm³ for C2, or developed ulcerations. Before injections, mice were randomized into treatment groups. Intratumoral injections of 30 μL PBS, d106S (1.5×10⁷ PFU/30 μL), or d106S-IL12 (1.5×10⁷ PFU/30 μL ) were performed every three days. For depletion experiments, mice were injected i.p. with 200 μL (100 μg) of anti-IFNAR (BioXCell Clone MAR1-5A3) starting on day seven every three days until day 34. Mice not receiving IFNAR blockade were rerandomized into treatment groups at day 40 and were injected i.p. with 200 μL (100 μg) of either anti-IFNγ (BioXCell Clone XMG1.2), anti-CD8 (BioXCell Clone 2.43), anti-CD4 (BioXCell Clone GK1.5), anti-NK1.1 (BioXCell Clone PK136), or isotype control antibodies (BioXCell Clone LTF-2) starting on day 40 every three days until day 61.

Cell Growth

B16^{Nectin1+;knockout} cell line growth was compared to B16^{Nectin1+;wildtype} cells using a Celigo image cytometer (Nexcelom 200-BFFL-5c). To compare IFNγ-mediated growth delay, wild-type or STAT1^{-/-} B16^{Nectin1} cells were plated in 96-well plates with stated amounts of IFNγ present during plating. Confluence measurements were made every 24 hours after plating in 96-well plates.

Flow Cytometry

To confirm absence of T cells during depletion, on day 62 mice were bled retrorbitally or spleens were harvested on day 63. Separately, spleens of sacrificed TCRα^{-/-} mice were compared to wild-type spleens to confirm genetic knockout. A hypotonic solution was used to lyse erythrocytes and the remaining cells were stained with flow cytometry antibodies for 20 minutes at 4° C. Cells were washed once with PBS and fixed with 1% formalin in PBS before analysis, which was performed on a Sony Biotechnology SP6800 Spectral Analyzer and analyzed with the Sony Biotechnology SP6800 Software and FlowJo (Tree Star, Ashland, OR). Flow cytometry antibodies used in this study were purchased from BioLegend: anti-CD11b (clone M1/70), anti-CD4 (clone GK1.5), anti-CD45 (clone 30-F11), anti-CD8 (clone 53-6.7), anti-NK1.1 (clone PK136), anti-TCRβ (H57-597), anti-I-A/I-E (MHC-II) (clone M5/114.15.2), and anti-Kb/Db (clone 28-8-6).

Statistical Analysis

All data were analyzed with GraphPad Prism. All data were presented as mean with S.E.M. errors bars. Significance was determined with a two-way ANOVA and Dunnett's multiple comparisons test for growth curves; unpaired t-tests for flow and IFNAR growth; Kaplan-Meier survival curves were analyzed using the log-rank test with a Bonferroni correction to adjust for multiple comparisons. Data were considered significant when P<0.05; *P<0.05, **P<0.01, ***P<0.001, ****P<0.0001.

1. A replication-defective oncolytic herpes simplex virus 1 (HSV-1) recombinant virus, comprising within its genome:
one or more therapeutic gene coding sequences,
wherein the genome comprises at least one alteration in each of a gene encoding infected cell polypeptides (ICP) 4, a gene encoding ICP22, a gene encoding ICP27 and a gene encoding ICP47, and
wherein the genome does not encode a functional ICP4, ICP22, ICP27 and ICP47 protein.

2. The recombinant virus of claim 1, wherein the therapeutic gene coding sequence is inserted in place of the U_{L}54 loci open reading frame 1 (ORF1).

3. The recombinant virus of claim 1, wherein the genome further comprises at least one alteration in an S component repeated sequence in a promoter between a gene encoding ICP4 and a gene encoding ICP22 or in a promoter between a gene encoding ICP4 and a gene encoding ICP47.

4. The recombinant virus of claim 1, wherein the genome lacks at least one Oct-1 site present in wild-type HSV-1 genome.

5. The recombinant virus of claim 1, wherein the genome lacks at least one Oct-1 site present in a promoter of a gene encoding ICP22 or a gene encoding ICP47.

6. The recombinant virus of claim 1, wherein the genome lacks at least one (e.g., one, two or three) SP1 binding site present in wild-type HSV-1 genome.

7. The recombinant virus of claim 6, wherein the genome lacks at least one (e.g., one, two or three) SP1 binding site present in a promoter of a gene encoding ICP22 or a gene encoding ICP47.

8. The recombinant virus of claim 1, wherein the therapeutic gene coding sequence is a codon optimized sequence.

9. The recombinant virus of claim 1, wherein the therapeutic gene coding sequence does not comprise an internal ribosome entry site (IRES).

10. The recombinant virus of claim 1, wherein the virus comprises two or more therapeutic gene coding sequences within its genome.

11. The recombinant virus of claim 10, wherein the virus comprises two or more therapeutic gene coding sequences within its genome and wherein at least two of the therapeutic gene coding sequences are different.

12. The recombinant virus of claim 1, wherein the therapeutic gene coding sequence encodes a cytokine.

13. The recombinant virus of claim 1, wherein the therapeutic gene coding sequence encodes a heterodimeric cytokine.

14. The recombinant virus of claim 1, wherein the therapeutic gene coding sequence encodes interleukin 12 (IL-12), IL-2, IL-3, IL-4, IL-5, IL-6, IL-7, IL-9, IL-10, IL-12, IL-15, IL-17, IL-18, interferon (IFN)-γ, or any combinations thereof.

15. The recombinant virus of claim 1, wherein the therapeutic gene coding sequence encodes a non-fusion protein.

16. The recombinant virus of claim 1, wherein the therapeutic gene coding sequence encodes a fusion protein.

17. The recombinant virus of claim 1, wherein the therapeutic gene coding sequence encodes a fusion protein comprising a first domain and a second domain linked via a linker, wherein the nucleotide sequence encoding the linker does not comprise an IRES.

18. The recombinant virus of claim 1, wherein the therapeutic gene coding sequence encodes a fusion protein comprising a first domain and a second domain linked via a linker, wherein one of the first and second domain is a p40 subunit of IL-12 and the other domain is a p35 subunit of IL-12.

19. The recombinant virus of claim 1, wherein the therapeutic gene coding sequence comprises a nucleotide sequence selected from the group consisting of SEQ ID NO:
14-SEQ ID NO: 24 and any combinations thereof.

20. The recombinant virus of claim 1, wherein the therapeutic gene coding sequence comprises a nucleic acid sequence encoding an amino acid sequence selected from the group consisting of SEQ ID NO: 14, SEQ ID NO: 17, SEQ ID NO: 19, SEQ ID NO:
21, SEQ ID NO: 22, and any combinations thereof

21. The recombinant virus of claim 1, wherein the recombinant virus has increased sensitivity to acyclovir relative to wild-type HSV-1.

22. A composition comprising a recombinant virus of claim 1.

23. A vaccine and/or immunomodulatory virus comprising a recombinant virus of claim 1.

24. A method of eliciting and/or modifying an immune response in a subject, the method comprising administering to said subject the recombinant virus of claim 1.

25. The method of claim 24, wherein the subject is diagnosed or has been diagnosed as having cancer.

26. A method of treating cancer, the method comprising: administering to the subject in need thereof the recombinant virus of claim 1.

27. The method of claim 26, wherein the cancer is a solid tumor, a benign tumor, or a malignant tumor.

28. The method of claim 26, wherein the cancer is selected from the group consisting of a carcinoma, a melanoma, a sarcoma, a germ cell tumor, and a blastoma.

29. The method of claim 26, wherein the cancer is metastatic.


SEQUENCE LISTING (30 sequences)

SEQ ID NO: 1 - protein, 775 aa, Human alphaherpesvirus 1
MEPRPGASTRRPEGRPQREPAPDVWVFPCDRDLPDSSDSEAETEVGGRGDADHHDDDSASEADSTDTELFETGLLGPQGVDGGAVSGGSPPREEDPGSCGGAPPREDGGSDEGDVCAVCTDEIAPHLRCDTFPCMHRFCIPCMKTWMQLRNTCPLCNAKLVYLIVGVTPSGSFSTIPIVNDPQTRMEAEEAVRAGTAVDFIWTGNQRFAPRYLTLGGHTVRALSPTHPEPTTDEDDDDLDDADYVPPAPRRTPRAPPRRGAAAPPVTGGASHAAPQPAAARTAPPSAPIGPHGSSNTNTTTNSSGGGGSRQSRAAAPRGASGPSGGVGVGVGVVEAEAGRPRGRTGPLVNRPAPLANNRDPIVISDSPPASPHRPPAAPMPGSAPRPGPPASAAASGPARPRAAVAPCVRAPPPGPGPRAPAPGAEPAARPADARRVPQSHSSLAQAANQEQSLCRARATVARGSGGPGVEGGHGPSRGAAPSGAAPLPSAASVEQEAAVRPRKRRGSGQENPSPQSTRPPLAPAGAKRAATHPPSDSGPGGRGQGGPGTPLTSSAASASSSSASSSSAPTPAGAASSAAGAASSSASASSGGAVGALGGRQEETSLGPRAASGPRGPRKCARKTRHAETSGAVPAGGLTRYLPISGVSSVVALSPYVNKTITGDCLPILDMETGNIGAYVVLVDQTGNMATRLRAAVPGWSRRTLLPETAGNHVMPPEYPTAPASEWNSLWMTPVGNMLFDQGTLVGALDFRSLRSRHPWSGEQGASTRDEGKQ

SEQ ID NO: 2 - DNA, 3233 bp, Human alphaherpesvirus 1
atggagccccgccccggagcgagtacccgccggcctgagggccgcccccagcgcgaggtgaggggccgggcgccatgtctggggcgccatgttggggggcgccatgttggggggcgccatgttgggggacccccgacccttacactggaaccggccgccatgttgggggacccccactcatacacgggagccgggcgccatgttggggcgccatgttagggggcgtggaaccccgtgacactatatatacagggaccgggggcgccatgttagggggcgcggaaccccctgaccctatatatacagggaccggggtcgccctgttaggggtcgccatgtgaccccctgactttatatatacagacccccaacacctacacatggcccctttgactcagacgcagggcccggggtcgccgtgggacccccctgactcatacacagagacacgcccccacaacaaacacacagggaccggggtcgccgtgttagggggcgtggtccccactgactcatacgcagggcccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcaggggggcaggactttgtgaggcggggggggagggggaactcgtgggcgctgattgacgcgggaaatccccccattcttacccgcccccccttttttcccctcagcccgccccggatgtctgggtgtttccctgcgaccgagacctgccggacagcagcgactcggaggcggagaccgaagtgggggggcggggggacgccgaccaccatgacgacgactccgcctccgaggcggacagcacggacacggaactgttcgagacggggctgctggggccgcagggcgtggatgggggggcggtctcgggggggagccccccccgcgaggaagaccccggcagttgcgggggcgccccccctcgagaggacggggggagcgacgagggcgacgtgtgcgccgtgtgcacggatgagatcgcgccccacctgcgctgcgacaccttcccgtgcatgcaccgcttctgcatcccgtgcatgaaaacctggatgcaattgcgcaacacctgcccgctgtgcaacgccaagctggtgtacctgatagtgggcgtgacgcccagcgggtcgttcagcaccatcccgatcgtgaacgacccccagacccgcatggaggccgaggaggccgtcagggcgggcacggccgtggactttatctggacgggcaatcagcggttcgccccgcggtacctgaccctgggggggcacacggtgagggccctgtcgcccacccaccctgagcccaccacggacgaggatgacgacgacctggacgacggtgaggcgggggggcggcgaggaccctgggggaggaggaggaggggggggggagggaggaataggcgggcgggcgggcgaggaaagggcgggccggggagggggcgtaacctgatcgcgccccccgttgtctcttgcagcagactacgtaccgcccgccccccgccggacgccccgcgcccccccacgcagaggcgccgccgcgccccccgtgacgggcggggcgtctcacgcagccccccagccggccgcggctcggacagcgcccccctcggcgcccatcgggccacacggcagcagtaacactaacaccaccaccaacagcagcggcggcggcggctcccgccagtcgcgagccgcggtgccgcggggggcgtctggcccctccgggggggttggggttgttgaagcggaggcggggcggccgaggggccggacgggcccccttgtcaacagacccgccccccttgcaaacaacagagaccccatagtgatcagcgactcccccccggcctctccccacaggccccccgcggcgcccatgccaggctccgccccccgccccggtccccccgcgtccgcggccgcgtcgggccccgcgcgcccccgcgcggccgtggccccgtgtgtgcgggcgccgcctccggggcccggcccccgcgccccggcccccggggcggagccggccgcccgccccgcggacgcgcgccgtgtgccccagtcgcactcgtccctggctcaggccgcgaaccaagaacagagtctgtgccgggcgcgtgcgacggtggcgcgcggctcgggggggccgggcgtggagggtggacacgggccctcccgcggcgccgccccctccggcgccgccccctccggcgcccccccgctcccctccgccgcctctgtcgagcaggaggcggcggtgcgtccgaggaagaggcgcgggtcgggccaggaaaacccctccccccagtccacgcgtccccccctcgcgccggcaggggccaagagggcggcgacgcaccccccctccgactcagggccgggggggcgcggccagggagggcccgggacccccctgacgtcctcggcggcctccgcctcttcctcctccgcctcttcctcctcggccccgactcccgcgggggccacctcttccgccaccggggccgcgtcctcctccgcttccgcctcctcgggcggggccgtcggtgccctgggagggagacaagaggaaacctccctcggcccccgcgctgcttctgggccgcgggggccgaggaagtgtgcccggaagacgcgccacgcggagacttccggggccgtccccgcgggcggcctcacgcgctacctgcccatctcgggggtctctagcgtggtcgccctgtcgccttacgtgaacaagacgatcacgggggactgcctgcccatcctggacatggagacggggaacatcggggcgtacgtggtcctggtggaccagacgggaaacatggcgacccggctgcgggccgcggtccccggctggagccgccgcaccctgctccccgagaccgcgggtaaccacgtgacgccccccgagtacccgacggcccccgcgtcggagtggaacagcctctggatgacccccgtggggaacatgctgttcgaccagggcaccctagtgggcgccctggacttccgcagcctgcggtctcggcacccgtggtccggggagcagggggcgtcgacccgggacgagggaaaacaataa

SEQ ID NO: 3 - protein, 1298 aa, Human alphaherpesvirus 1
MASENKQRPGSPGPTDGPPPTPSPDRDERGALGWGAETEEGGDDPDHDPDHPHDLDDARRDGRAPAAGTDAGEDAGDAVSPRQLALLASMVEEAVRTIPTPDPAASPPRTPAFRADDDDGDEYDDAADAAGDRAPARGREREAPLRGAYPDPTDRLSPRPPAQPPRRRRHGRWRPSASSTSSDSGSSSSSSASSSSSSSDEDEDDDGNDAADHAREARAVGRGPSSAAPAAPGRTPPPPGPPPLSEAAPKPRAAARTPAASAGRIERRRARAAVAGRDATGRFTAGQPRRVELDADATSGAFYARYRDGYVSGEPWPGAGPPPPGRVLYGGLGDSRPGLWGAPEAEEARRRFEASGAPAAVWAPELGDAAQQYALITRLLYTPDAEAMGWLQNPRVVPGDVALDQACFRISGAARNSSSFITGSVARAVPHLGYAMAAGRFGWGLAHAAAAVAMSRRYDRAQKGFLLTSLRRAYAPLLARENAALTGAAGSPGAGADDEGVAAVAAAAPGERAVPAGYGAAGILAALGRLSAAPASPAGGDDPDAARHADADDDAGRRAQAGRVAVECLAACRGILEALAEGFDGDLAAVPGLAGARPASPPRPEGPAGPASPPPPHADAPRLRAWLRELRFVRDALVLMRLRGDLRVAGGSEAAVAAVRAVSLVAGALGPALPRDPRLPSSAAAAAADLLFDNQSLRPLLAAAASAPDAADALAAAAASAAPREGRKRKSPGPARPPGGGGPRPPKTKKSGADAPGSDARAPLPAPAPPSTPPGPEPAPAQPAAPRAAAAQARPRPVAVSRRPAEGPDPLGGWRRQPPGPSHTAAPAAAALEAYCSPRAVAELTDHPLFPVPWRPALMFDPRALASIAARCAGPAPAAQAACGGGDDDDNPHPHGAAGGRLFGPLRASGPLRRMAAWMRQIPDPEDVRVVVLYSPLPGEDLAGGGASGGPPEWSAERGGLSCLLAALANRLCGPDTAAWAGNWTGAPDVSALGAQGVLLLSTRDLAFAGAVEFLGLLASAGDRRLIVVNTVRACDWPADGPAVSRQHAYLACELLPAVQCAVRWPAARDLRRTVLASGRVFGPGVFARVEAAHARLYPDAPPLRLCRGGNVRYRVRTRFGPDTPVPMSPREYRRAVLPALDGRAAASGTTDAMAPGAPDFCEEEAHSHAACARWGLGAPLRPVYVALGREAVRAGPARWRGPRRDFCARALLEPDDDAPPLVLRGDDDGPGALPPAPPGIRWASATGRSGTVLAAAGAVEVLGAEAGLATPPRREVVDWEGAWDEDDGGAFEGDGVL

SEQ ID NO: 4 - DNA, 3885 bp, Human alphaherpesvirus 1
atggcgtcggagaacaagcagcgccccggctccccgggccccaccgacgggccgccgcccaccccgagcccagaccgcgacgagcggggggccctcgggtggggcgcggagacggaggagggcggggacgaccccgaccacgaccccgaccacccccacgacctcgacgacgcccggcgggacgggagggcccccgcggcgggcaccgacgccggcgaggacgccggggacgccgtctcgtcgcgacagctggctctgctggcctccatggtagaggaggccgtccggacgatcccgacgcccgaccccgcggcctcgccgccccggacccccgcctttctagccgacgacgatgacggggacgagtacgacgacgcagccgacgccgccggcgaccgggccccggcccggggccgcgaacgggaggccccgctacgcggcgcgtatccggaccccacggaccgcctgtcgccgcgcccgccggcccagccgccgcggagacgtcgtcacggccggcggcggccatcggcgtcatcgacctcgtcggactccgggtcctcgtcctcgtcgtccgcatcctcttcgtcctcgtcgtccgacgaggacgaggacgacgacggcaacgacgcggccgaccacgcacgcgaggcgcgggccgtcgggcggggtccgtcgagcgcggcgccggaagcccccgggcggacgccgcccccgcccgggccaccccccctctccgaggccgcgcccaagccccgggcggcggcgaggacccccgcggcctccgcgggccgcatcgagcgccgccgggcccgcgcggcggtggccggccgcgacgccacgggccgcttcacggccgggcagccccggcgggtcgagctggacgccgacgcggcctccggcgccttctacgcgcgctatcgcgacgggtacgtcagcggggagccgtggcccggcgccgggcccccgcccccggggcgggtgctgtacggcggcctgggcgacagccgcccgggcctctggggggcgcccgaggcggaggaggcgcgacgccggttcgaggcctcgggcgccccggcggccgtgtgggcgcccgagctgggcgacgccgcgcagcagtacgccctgatcacgcggctgctgtacaccccggacgcggaggccatggggtggctccagaacccgcgcgtggtccccggggacgtggcgctggaccaggcctgcttccggatctcgggcgccgcgcgcaacagcagctccttcatcaccggcagcgtggcgcgggccgtgccccacctgggctacgccatggcggccggccgcttcggctggggcctggcgcacgcggcggccgccgtggccatgagccgccgatacgaccgcgcgcagaagggcttcctgctgaccagcctgcgccgcgcctacgcgcccctgttggcgcgcgagaacgcggcgctgacgggggccgcggggagccccggcgccggcgcagatgacgagggggtcgccgccgtcgccgccgccgcaccgggcgagcgcgcggtgcccgccgggtacggcgccgcggggatcctcgccgccctggggcggctgtccgccgcgcccgcctcccccgtggggggcgacgaccccgacgccgcccgccacgccgacgccgacgccgggcgccgcgcccaggccggccgcgtggccgtcgagtgcctggccgcctgccgcgggatcctggaggcgctggccgagggcttcgacggcgacctggcggccgtcccggggctggccggggcccggcccgccagccccccgcggccggagggacccgcgggccccgcttccccgccgccgccgcacgccgacgcgccccgcctgcgcgcgtggctgcgcgagctgcggttcgtgcgcgacgcgctggtgctcatgcgcctgcgcggggacctgcgcgtggccggcggcagcgaggccgccgtggccgccgtgcgcgccgtgagcctggtcgccggggccctgggccccgcgctgccgcgggacccgcgcctgccgagctccgcggccgccgccgccgcggacctgctgtttgagaaccagagcctccgccccctgctggcggcggcggccagcgcaccggacgccgccgacgcgctggcggccgccgccgcctccgccgcgccgcgggaggggcgcaagcgcaagagtcccggcccggcccggccgcccggaggcggcggcccgcgacccccgaagacgaagaagagcggcgcggacgcccccggctcggacgcccgcgcccccctccccgcgcccccctccacgcccccggggcccgagcccacccccgcccagcccgcggcggcccggggcgccgcggcgcaggcccgcccgcgccccgtggcgctgtcgcgccggcccgccgagggccccgaccccctgggcggctggcggcggcagccccgggggcccagccacacggcggcgcccgcggccgccgccctggaggcctactgctccccgcgcgccgtggccgagctcacggaccacccgctgttccccgtcccctggcgaccggccctcatgtttgacccgcgggccctggcctcgatcgccgcgcggtgcgccgggcccgcccccgccgcccaggccgcgtgcggcggcgacgacgacgagaacccccacccccacggggccgccgggggccgcctctttggccccctgcgcgcctcgggcccgctgcgccgcatggcggcctggatgcgccagatccccgaccccgaggacgtgcgcgtggtggtgctgtactcgccgctgccgggcgaggacctggccggcggcggggcctcgggggggccgccggagtggtccgccgagcgcggcgggctgtcctgcctgctggcggccctggccaaccggctgtgcgggccggacacggccgcctgggcgggcaactggaccggcgcccccgacgtgtcggcgctgggcgcgcagggcgtgctgctgctgtccacgcgggacctggccttcgccggggccgtggagtttctggggctgctcgccagcgccggcgaccggcggctcatcgtggtcaacaccgtgcgcgcctgcgactggcccgccgacgggcccgcggtgtcgcggcagcacgcctacctggcgtgcgacctgctgcccgccgtgcagtgcgccgtgcgctggccggcggcgcgggacctgcgccgcacggtgctggcctcgggccgcgtgttcggcccgggggtcttcgcgcgcgtggaggccgcgcacgcgcgcctgtaccccgacgcgccgccgctgcgcctgtgccgcggcggcaacgtgcgctaccgcgtgcgcacgcgcttcggcccggacacgccggtgcccatgtccccgcgcgagtaccgccgggccgtgctgccggcgctggacggccgggcggcggcctcggggaccaccgacgccatggcgcccggcgcgccggacttctgcgaggaggaggcccactcgcaccgcgcctgcgcgcgctggggcctgggcgcgccgctgcggcccgtgtacgtggcgctggggcgcgaggcggtgcgcgccggcccggcccggtggcgcgggccgcggagggacttttgcgcccgcgccctgctggagcccgacgacgacgcccccccgctggtgctgcgcggcgacgacgacgacggcccgggggccctgccgccggcgccgcccgggattcgctgggcctcggccacgggccgcagcggcaccgtgctggcggcggcgggggccgtggaggtgctgggggcggaggcgggcttggccacgcccccgcgacgggacgttgtggactgggaaggcgcctgggacgaagacgacggcggcgcgttcgagggggacggggtgctgtaa

SEQ ID NO: 5 - protein, 420 aa, Human alphaherpesvirus 1
MADISPGAFAPCVKARRPALRSPPLGTRKRKRPSRPLSSESEVESDTALESEVESETASDSTESGDQDEAPRIGGRRAPRRLGGRFFLDMSAESTTGTETDASVSDDPDDTSDWSYDDIPPRPKRARVNLRLTSSPDRRDGVIFPKMGRVRSTRETQPRAPTPSAPSPNAMLRRSVRQAQRRSSARWTPDLGYMRQCINQLFRVLRVARDPHGSANRLRHLIRDCYLMGYCRARLAPRTWCRLLQVSGGTWGMHLRNTIREVEARFDATAEPVCKLPCLETRRYGPECDLSNLEIHLSATSDDEISDATDLEAAGSDHTLASQSDTEDAPSPVTLETPEPRGSLAVRLEDEFGEFDWTPQEGSQPWLSAVVADTSSVERPGPSDSGAGRAAEDRKCLDGCRKMRFSTACPYPCSDTFLRP

SEQ ID NO: 6 - DNA, 2831 bp, Human alphaherpesvirus 1
ggccaccgccgcgcgggcccggcggcgctcgatgcggcccgcggaggccgcgggggtcctcgccgccgcccggggcttgggcgcggcctcggagagggggggtggcccgggcgggggcggcgtccgcccgggggcttccggcgccgcgctcgacggaccccgcccgacggcccgcgcctcgcgtgcgtggtcggccgcgtcgttgccgtcgtcgtcctcgtcctcgtcggacgacgaggacgaagaggatgcggacgacgaggacgaggacccggagtccgacgaggtcgatgacgccgatggccgccgccggccgtgacgacgtctccgcggcggctgggccggcgggcgcggcgacaggcggtccgtggggtccggatacgcgccgcgtagcggggcctcccgttcgcggccccgggccggggcccggtcgccggcggcgtcggctgcgtcgtcgtactcgtccccgtcatcgtcgtcggctagaaaggcgggggtccggggcggcgaggccgcggggtcgggcgtcgggatcgtccggacggcctcctctaccatggaggccagcagagccagctgtcgcgacgagacggcgtccccggcgtcctcgccggcgtcggtgcccgccgcgggggccctcccgtcccgccgggcgtcgtcgaggtcgtgggggtggtcggggtcgtggtcggggtcgtccccgccctcctccgtctccgcgccccacccgagggccccccgctcgtcgcggtctgggctcggggtgggcggcggcccgtcggtggggcccggggagccggggcgctgcttgttctccgacgccatcgccgatgcggggcgatcctccggggatacggctgcgacggcggacgtagcacggtaggtcacctacggactctcgatggggggagggggcgagacccacggaccccgacgacccccgccgtcgacgcggaactagcgcggaccggtcgatgcttgggtggggaaaaaggacagggacggccgatccccctcccgcgcttcgtccgcgtatcggcgtcccggcgcggcgagcgtctgacggtctgtctctggcggtcccgcgtcgggtcgtggatccgtgtcggcagccgcgctccgtgtggacgatcggggcgtcctcgggctcatatagtcccaggggccggcgggaaggaggagcagcggaggccgccggccccccgccccccggcgggcccaccccgaacggaattccattatgcacgaccccgccccgacgccggcacgccgggggcccgtggccgcggcccgttggtcgaacccccggccccgcccatccgcgccatctgccatggacggggcgcgagggcgggtgggtccgcgccccgccccgcatggcatctcattaccgcccgatccggcggtttccgcttccgttccgcatgctaacgaggaacgggcagggggcggggcccgggccccgacttcccggttcggcggtaatgagatacgagccccgcgcgcccgttggccgtccccgggcccccggtcccgcccgccggacgccgggaccaacgggacggcgggcggcccttgggccgcccgccttgccgcccccccattggccggcgggcgggaccgccccaagggggcggggccgccgggtaaaagaagtgagaacgcgaagcgttcgcacttcgtcccaatatatatatattattagggcgaagtgcgagcactggcgccgtgcccgactccgcgccggccccgggggcggacccgggcggcggggggcgggtctctccggcgcacataaaggcccggcgcgaccgacgcccgcagacggcgccagccacgaacgacgggagcggctgcggagcacgcggaccgggagcgggagtcgcagagggccgtcggagcggacggcgtcggcatcgcgacgccccggctcgggatcgggatcgcatcggaaagggacacgcggacgcgggggggaaagacccgcccaccccacccacgaaacacaggggacgcaccccgggggcctccgacgacagaaacccaccggtccgccttttttgcacgggtaagcaccttgggtgggcagaggaggggggacgcgggggcggaggaggggggacgcgggggcggaggaggggggacgcgggggcggaggaggggggacgcgggggcggaggaggggggacgcgggggcggaggagggggctcacccgcgttcgtgccttcccgcaggaggaacgccctcgtcgaggcgaccggcggcgaccgttgcgtggaccgcttcctgctcgtcgggggggggggagccactgtggtcctccgggacgttttctggatggccgacatttccccaggcgcttttgtgccttgtgtaaaagcgcggcgtcccgctctccgatccccgcccctgggcacgcgcaagcgcaagcgccctgcccgccccctctcatcggagtctgaggtcgaatccgagacagccttggagtctgaggtcgaatccgagacagcatcggattcgaccgagtctggggaccaggaggaagccccccgcatcggtggccgtagggccccccggaggcttggggggcggttttttctggacatgtcggcggaatccaccacggggacggaaacggatgcgtcggtgtcggacgaccccgacgacacgtccgactggtcttgtgacgacattcccccacgacccaagcgggcccgggtaaacctgcggctcactagctctcccgatcggcgggatggggttatttttcctaagatggggcgggtccggtctacccgggaaacgcagccccgggcccccaccccgtcgg

SEQ ID NO: 7 - protein, 512 aa, Human alphaherpesvirus 1
MATDIDMLIDLGLDLSDSDLDEDPPEPAESRRDDLESDSSGECSSSDEDMEDPHGEDGPEPILDAARPAVRPSRPEDPGVPSTQTPRPTERQGPNDPQPAPHSVWSRLGARRPSCSPEQHGGKVARLQPPPTKAQPARGGRRGRRRGRGRGGPGAADGLSDPRRRAPRTNRNPGGPRPGAGWTDGPGAPHGEAWRGSEQPDPPGGQRTRGVRQAPPPLMTLAIAPPPADPRAPAPERKAPAADTIDATTRLVLRSISERAAVDRISESFGRSAQVMHDPFGGQPFPAANSPWAPVLAGQGGPFDAETRRVSWETLVAHGPSLYRTFAGNPRAASTAKAMRDCVLRQENFIEALASADETLAWCKMCIHHNLPLRPQDPIIGTTAAVLDNLATRLRPFLQCYLKARGLCGLDELCSRRRLADIKDIASFVFVILARLANRVERGVAEIDYATLGVGVGEKMHFYLPGACMAGLIEILDTHRQECSSRVCELTASHIVAPPYVHGKYFYCNSLF

SEQ ID NO: 8 - DNA, 1539 bp, Human alphaherpesvirus 1
atggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttag

SEQ ID NO: 9 - protein, 88 aa, Human alphaherpesvirus 1
MSWALEMADTFLDTMRVGPRTYADVRDEINKRGREDREAARTAVHDPERPLLRSPGLLPEIAPNASLGVAHRRTGGTVTDSPRNPVTR

SEQ ID NO: 10 - DNA, 267 bp, Human alphaherpesvirus 1
tcaacgggttaccggattacggggactgtcggtcacggtcccgccggttcttcgatgtgccacacccaaggatgcgttgggggcgatttcgggcagcagcccgggagagcgcagcaggggacgctccgggtcgtgcacggcggttctggccgcctcccggtcctcacgcccccttttattgatctcatcgcgtacgtcggcgtacgtcctgggcccaacccgcatgttgtccaggaaggtgtccgccatttccagggcccacgacat

SEQ ID NO: 11 - DNA, 271 bp, Human alphaherpesvirus 1
aattccattatgcacgaccccgccccgacgccggcacgccgggggcccgtggccgcggcccgttggtcgaacccccggccccgcccatccgcgccatctgccatggacggggcgcgagggcgggtgggtccgcgccccgccccgcatggcatctcattaccgcccgatccggcggtttccgcttccgttccgcatgctaacgaggaacgggcagggggcggggcccgggccccgacttcccggttcggcggtaatgagatacgagccccgc

SEQ ID NO: 12 - DNA, 271 bp, Human alphaherpesvirus 1
cgcgcggggctcgtatctcattaccgccgaaccgggaagtcggggcccgggccccgccccctgcccgttcctcgttagcatgcggaacggaagcggaaaccgccggatcgggcggtaatgagatgccatgcggggcggggcgcggacccacccgccctcgcgccccgtccatggcagatggcgcggatgggcggggccgggggttcgaccaacgggccgcggccacgggcccccggcgtgccggcgtcggggcggggtcgtgcataatgga

SEQ ID NO: 13 - DNA, 1474 bp, Human alphaherpesvirus 1
cgccgatgcggggcgatcctccggggatacggctgcgacggcggacgtagcacggtaggtcacctacggactctcgatggggggagggggcgagacccacggaccccgacgacccccgccgtcgacgcggaactagcgcggaccggtcgatgcttgggtggggaaaaaggacagggacggccgatccccctcccgcgcttcgtccgcgtatcggcgtcccggcgcggcgagcgtctgacggtctgtctctggcggtcccgcgtcgggtcgtggatccgtgtcggcagccgcgctccgtgtggacgatcggggcgtcctcgggctcatatagtcccaggggccggcgggaaggaggagcagcggaggccgccggccccccgccccccggcgggcccaccccgaacggaattccattatgcacgaccccgccccgacgccggcacgccgggggcccgtggccgcggcccgttggtcgaacccccggccccgcccatccgcgccatctgccatggacggggcgcgagggcgggtgggtccgcgccccgccccgcatggcatctcattaccgcccgatccggcggtttccgcttccgttccgcatgctaacgaggaacgggcagggggcggggcccgggccccgacttcccggttcggcggtaatgagatacgagccccgcgcgcccgttggccgtccccgggcccccggtcccgcccgccggacgccgggaccaacgggacggcgggcggcccttgggccgcccgccttgccgcccccccattggccggcgggcgggaccgccccaagggggcggggccgccgggtaaaagaagtgagaacgcgaagcgttcgcacttcgtcccaatatatatatattattagggcgaagtgcgagcactggcgccgtgcccgactccgcgccggccccgggggcggacccgggcggcggggggcgggtctctccggcgcacataaaggcccggcgcgaccgacgcccgcagacggcgccagccacgaacgacgggagcggctgcggagcacgcggaccgggagcgggagtcgcagagggccgtcggagcggacggcgtcggcatcgcgacgccccggctcgggatcgggatcgcatcggaaagggacacgcggacgcgggggggaaagacccgcccaccccacccacgaaacacaggggacgcaccccgggggcctccgacgacagaaacccaccggtccgccttttttgcacgggtaagcaccttgggtgggcagaggaggggggacgcgggggcggaggaggggggacgcgggggcggaggaggggggacgcgggggcggaggaggggggacgcgggggcggaggaggggggacgcgggggcggaggagggggctcacccgcgttcgtgccttcccgcaggaggaacgccctcgtcgaggcgaccggcggcgaccgttgcgtggaccgcttcctgctcgtcggg

SEQ ID NO: 14 - DNA, 7185 bp, Homo sapiens
atttcgctttcattttgggccgagctggaggcggcggggccgtcccggaacggctgcggccgggcaccccgggagttaatccgaaagcgccgcaagccccgcgggccggccgcaccgcacgtgtcaccgagaagctgatgtagagagagacacagaaggagacagaaagcaagagaccagagtcccgggaaagtcctgccgcgcctcgggacaattataaaaatgtggccccctgggtcagcctcccagccaccgccctcacctgccgcggccacaggtctgcatccagcggctcgccctgtgtccctgcagtgccggctcagcatgtgtccagcgcgcagtgagtactcagcccgccaggtctttggctcgctcgggtgcggaggggcggctgcttgggaagagtgggtagaaactcaagtctgcctaaggagagactggaacagcagcagccgtctcctgcaaagaggaataagaataggggtctcaccgtgtgctaaatagggcatgtctcttttcatctcgaaacagcctgatgagctatcattatgcccacttcgtagaggagaaactgaggcccagcgtgggagatttcctgctttcctgcctagggtccctcagctacaaacagaaggcagatcctagccagttctaaagtcaggcttggccgggtgcaatggctcacgcctgtaatcccagcactttgggaggccaaggcggacagatctcctgaggtcaggagttcaagaccagcctgaccaacatggtgaaaccccgtctctactaacaaatacacaaattagccgagtgtggtggtgggtgcctgtaatcccatctactcgggagtctaaggcagggaaaattgcttgaactcggaggtggaggttgcagtgagccaaaatcgtgccactgcactccagtctgggccacagagtgagactccgtcttaaaaaaacaaaaaaagtcaggctttctcactccactgtttttaaaacactgggtcccaagtctgactcagccacttcaccacctggtctgggtttccctgatcaaaaacatgtaggctttctctgggtgagtgttgggttcaacacccttgtccaagctcatctctagaccctcacagggagctggcttctagccctagaatagagtggcgctttgtaataactcgagtcatctctcaggtgttgaaggaaaagtgttggaaatgggttgagggaggtgggtgccaagcatgaatggatgggtgaaggtggccagaatggagggaaggtggtgcaggcaggccatccaggctgaagctcctccacctgctcctcttccttccaggcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggtgagcctttcctgtcctctccactgtggacctgcaccctccctgaggaaggggcctctgatcctcccctctggtacctgatggaactgcagagaaattgtggaagttcattagcagctgtcaacagcaggagagggaactttacaaatggcccaagtgttaaagagtcctagtgaaattgtgtctccagagaaagcaagagtagtaataaattataatgatgttggttttggctatgtctacactgagtaaagtggatatttgcagtgttcctgtagcctgccaacagagatagaattgtgtcaggtccacatggtttctctggcaccacaccactgatcaatcccggaaatagttacttgggtgcatactctgtgttggggggcaggggtacaaagatgaaatagccttgtccctcctgctgcccaccagcttctacttggtgacacagttccttcttggagcaacacatctcagggaggactatgacagtgcacatgcagccttcattttctagggaagagttacctaattttatttacagcttcgttcttttaggtttcatattttaatgtaaaaaacattgccgttaaaaactgctccagtagctactgctgcaaaggctaaaggctaatgttttaaaagatgtcctctgcctttctgctctctaggaattttttcttgagtattttctagcttgttgccttaggaatatcttaaaataagctcaatgccaccaagccttcttaaagggctcctttcccctttcaatctttcataatgtgctgtgcattgctcctcacaattcaattaatcttaattggacagggggctcaagtgaaaactgctttgtttcccgaagaaaggttcaaaatgggtaacttttaggtgctccaattcatatagatttttttgtgcaaaagctgacacccctactcccagatatagtcctaagggtcaaaagattatagaaatcaatttaatgttttgtacatcatttgtcaaatttgctgtagtagaaaacaaatgaggtagatgcaagtgattcaccggccatatccagggcacaggtttgtagaatttgggctatatgtttattttttagtttgatgccattcaaaaaccaaacatttcaattaagatttcaaaattctagcttctcttgaaaagatctgaagaacaacactggactcacacctccaaatctaactctttataacctgctcagaatagtggggttgcccaggtctgtttaaacacctccgggaatagtgtattcatttcctactgctgctgtcacaaatgtagtgggtttaaatgacacaaatttattatcttacagctctgggcatcagaagtccaaaatgggtcctactgggctaaagtcaatgtgtcagcagagctgtagccctttctggaggctcttggggagaatctgttgccttgctttgtcaggtctagaggctgcccacattccttgacttgtggccccttccattgtccataccagcaatgtctggttgagtctttctcatgctgctccctctggactgaccttctacccgcttctacttttgaggacactgcttacactggattcacccagataatccaggatcatctctctactcttagatcagctgattagcaaattgaatgtcatctgcaccttcattcctctttggcaggtcacatagctcattaccgggttccagggattgggaggtggatgtctttggaggtggagaattggtctagctaccacagaaagggattttactacttgtgcccaaacgtattctgaattttcaggccttgcttcttggctcatgctgttacctctgtctgaaatttccctcctccattgtcgacactttactcctcttaatgtgggtgtcccaactctaggaagctttgcttcccttcctgcccctcctcagaagcttcatgcccttctcaccatgtctcacctactgatgcctcctggtcatttgggcaattgtctgtctcagcagctaaacttgcaatccaggagggtatacatggtcctcttctctgtcatgttcttagtacctaatggagtgcctgccacccattaggctgttgaatggaaaaatgatcgtaatcttcaatcatacagtcctttacttcctaagatacatttcataattttacccaacggtgctttccaacacactaagatgacatctctgtgtatgtgtgtgcgtgtgtgcactgcaaattaaatctcagcttgtcttaagggtttgcatgtttgttatatccatcagactgtgaccttaacagttccccctttagaagttatacttgaaataggtattcatagaatacagtcactttagcaaaagaagaaacaattttcaccagaagcaagtgtcacagtgaggtgtggaggctggttagcacaggctgttgacatgatttattgtgtttacataatgaaaaaatatgtaatccagagaaacatgccccaagcttagggagaggagggtgagagacagaatgtaaggaatctattttcttttgtattatgaagaaccagaatttcctgcaagtcaaagtgacagaggtcaagggcagccagccggagccttcctggcaccctggcttaccagccttgtggggtgccaggtgcattatcaatgttataaactgagtttctccttcattttttataggccagacaaactctagaattttacccttgcacttctgaagagattgatcatgaagatatcacaaaagataaaaccagcacagtggaggcctgtttaccattggaattaaccaaggtataaaggattttcctcccagagcatgcagtgtggttaaaaactgtgcatcaaatctcacctgcttctaaaaattcacgtttctggtatccatcattatgggattttaactggtcctacttcagaagttagatttgggagaaaaattatttttaaaaaatggttttttttgcaacaatgtgaatacacttaacacttaaaaatagttcagatgctaaatattatgatatgtgtttttaccataataaaaaaaatttgagacctgaagagtcagaaagatttatactaagaattaatactttgatatatgattttttccctctagaatgagagttgcctaaattccagagagacctctttcataactgtaagtcaaaaaatgaaaagtttcagcctgtatgatgaattcatatcactgatgtctgattattttttcctctagaatgggagttgcctggcctccagaaagacctcttttatgatggtaagacacacagctctttcctcaaatgcaatgggggaaatgtttttagcccatctcaatggatacttccccatcttgtcatgtcacccaggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggtaagacttcattctatcagtgagagcacctttttcatgctaaagataaccagccagggtctttgataaagagatataaaaagaggtctggaggccttttaaaggcctgacagacctacattttcaagaagacagccttgagggtgccgtctatagggagcacaaatgtgagcagatcacattatgaaaagcagactccaaagtattcactctgtggtatcccccacactcggcaaatgtttttgtgcattttcttatgtcagcctcaaaacaaccatatgggatctgcacaaaggagggaaccaaaccttaggagagttaaaccacttgtctgaggctctgcctcttaaaatccttcaagaggatttcactacacttaccttctcactcacctctaaaggctccaggacgtgctccaggatgtgcatgcaaggggccagtttgcatatctgcaaatattgctgggaaacaagcaagattggtgcctgattatgacccattgtgaaccaaaatgtgaaccaaataaaagaatgacctatcatctggggcatctataatgttattcataggcaagaacttgctttgttatgttctgaatcaccataacacagatgctaatataaaacaaatatttatataagcgagggtgactgctttggtgacgaggacatgggataaaaatatggtggcagaaatcattgtctgaaaagtaattgttttacttttattcttttcgtgtgtgtgtgtgtgtgtgtgtgtgtgtgtgtgtgtgtgcatgtgccagatttcttgtttgaaaggcaatgagcttcatccaagtatcaaagaatgttagcatctagagagctgtagttgctatttcatttttaggaccaagagttgggtgatttgggtgctagaatcaattctaccagtaaccagacaactattcccaagtcacttaacccctctgtgcctcagtttcctccagtataaaatggggtgactttattctagctttcttttagactttttgtgaggaagatatgaaagtatttattcatcaagggtgcaaatgtaaggttttatattctgttatcaaatcaaagtgctaaacttgggaaattcattgccaggtttatctgacacaaatggcatgtcttcagtaaacaggcctgttactgatagtgagttctgatcaatagcaatccatcacctccctgtgctaaacagaagtgggctttttaatgtaacatatataaaattaattagatattgcagcagatgtcattttaaaggaactgtttctttctaagacacaactcccactgatgattttttctaaatagttttaagggtcttttcagagctcattgaagatggatgtgcttggaaaatgagtatttcttttctcattctgcctggtgatctggctgagagtagatttggattgggtttaggagtggcataagggactgagttgcaggctctgagacatgtactggcttcactcatttttatgaatgaatatttgaattttggaataccatgtaagtcatgcttactgttcattctcctaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggagggggaaagaaggatgggactattacatccacatgatacctctgatcaagtatttttgacatttactgtggataaattgtttttaagttttcatgaatgaattgctaagaagggaaaatatccatcctgaaggtgtttttcattcactttaatagaagggcaaatatttataagctatttctgtaccaaagtgtttgtggaaacaaacatgtaagcataacttattttaaaatatttatttatataacttggtaatcatgaaagcatctgagctaacttatatttatttatgttatatttattaaattatttatcaagtgtatttgaaaaatatttttaagtgttctaaaaataaaagtattgaattaaagtga

SEQ ID NO: 15 - DNA, 1444 bp, Homo sapiens
atttcgctttcattttgggccgagctggaggcggcggggccgtcccggaacggctgcggccgggcaccccgggagttaatccgaaagcgccgcaagccccgcgggccggccgcaccgcacgtgtcaccgagaagctgatgtagagagagacacagaaggagacagaaagcaagagaccagagtcccgggaaagtcctgccgcgcctcgggacaattataaaaatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggagggggaaagaaggatgggactattacatccacatgatacctctgatcaagtatttttgacatttactgtggataaattgtttttaagttttcatgaatgaattgctaagaagggaaaatatccatcctgaaggtgtttttcattcactttaatagaagggcaaatatttataagctatttctgtaccaaagtgtttgtggaaacaaacatgtaagcataacttattttaaaatatttatttatataacttggtaatcatgaaagcatctgagctaacttatatttatttatgttatatttattaaattatttatcaagtgtatttgaaaaatatttttaagtgttctaaaaataaaagtattgaattaaagtga

SEQ ID NO: 16 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 17 - DNA, 15708 bp, Homo sapiens
tatgattacaaagaagagtttttattagttcagcctcagaatgcaaaaataaataaataaataaacaaacaggaaacaaatgtaatcactttacagagcgcacatacattacttaaaagtagcaccttcatggagccatattttctggtcataattgtgtatcaggttcattcatgctaatgagaaagggattccagattttctttgcatctgtctgcttctcacagggctgttaagaagccacctgccattctgacaatttcatgtccttagccataactacttgtcctctctcttgaatcttaagatctttttgccttccagacacttacggtgtttctgtgtcatcctcctgtgtcttttagagaggtgggggtgaggactgccatggaagctaaagctgaattttaatttcaatcatttttttttttgagacagggtctcattctgtcacctaggctggtatgcagtggcacaatcacggctcaccatagccttgacctcctgggctcaagagatcctcctgcctcatcctcctgaacagctgggaccacaagcatgagccaccacgcctggctaattttaaattttttttgtagagacggggtcttgttatgtttcccaggctggtcaatcatttttttctagcccttttaaaattcaggcatccaaagattaaacttgcttagaagtgtaagtggccctaaattgctttatcaacaccatctccaggaagtcttcacagacatgggaactagcatcttgttctcctggatcttctcaacaggtttgcattgtcaggtttccatgtaagtatctcttgcgttcccatccatcacaagtgtatgatggcactggtatcagaacactgcattcttcctgattgtcataaaactgatgtacttgcagccttgcttgaaaagttgtcagtacaaataaaattaaattcacattttgcataatagggactgatcctgatggatcaggtcataagagtatgaaacattccatacatcctggcagacaaacgttaaataacagtaatatactaataaatacataaattacttaaatatttaaatagcatgaaggcccatggcaacttgagagctggaaaatctatacataaattagctgattgtttcaatgagcatttagcatctaactatacaaatacagcaaagatatcattgtgatcctaaaaaaacgttttaaagcaaatcagatagaaattatctttttgggtctattccgttgtgtctttaaacattttgcttaatatcttccacttttcctccaaattttcatcctggatcagaacctggaagagaatgccaaaagttgatgtggggtgacattgtaacagcaatgtctcttcttatttctcacaacatatgatcctgggcaactgggtttcagggatttcatgccagaaggcccaggccttccttatgtggcctggaatttggctggcaccatgcttgccagaggctttcttgagggttttcttaaaataatatctgattgtgttacttccttgctgaaaacccttcagtgggtttcagggcccggggcccccagaacaagattctgagtcctgcaagcttgcaagtcctccatgctctgcctcctggctacctctctcttttctttgcctttctctttaggaggccagaaccccggtctgttttctttcctgcaatatccctgtggccagcacagtgtcctacataacaaaggcaccaaataaatatctgttagtgaataaatgtatgtttctgattctggcaactgggtgctggccactccatccccctttcctctccaacacagcccccaatcatatccctgcatccaggtgcactgagagtgcaggcctgggctggcctttgagggcctgctcacctaactgcagggcacagatgcccattcgctccaagatgagctatagtagcggtcctgggcccgcacgctaatgctggcatttttgcggcagatgaccgtggctgaggtcttgtccgtgaagactctatctttctgcaaaagagaaggaaagctgtgaagaccccttggcaacatagtcacagggtaagctgagcctgtttctgcaatgcatactctcccaaaacaagcccatcttggtcttagggcactgtgcttgcaattcacaggggtgtgcatagaacttgcaccacctactggcagacactccacatgtaggtgcagcttttgtactttgtaagcccttgagggagaaatcgtttggcccagctttcatctttctagcacaattgccttgcctcaagaccccatggccactcacccagtcctgagctgatggtgagagagagacagatttgctctagccatccctgagatattgagaaataccatatcctgatcatttcatcagaaaacttgccttcaaattctggcactgctacttaatagctgtgtaacttcaggaaaatgtcttaggttctctgtgtctgtttcctcacttataaatagggataacaataatgcctacttcatagaattatagttcaaggtaaaaatcacgtcaaactcttagcaagtctttagcacataggaagcactcaatatcacctattagtcatacagatcttaaatagggaaagtacttgccaagatgtaaaataatatttaggtaaatatctattccaggatagcctccctacctaattattttcccagagagtaactagctcactgaatttctaccacatgctaaatgctatgctgaattagggctttgtccagtgattttaaaagtggggtgaaaggagtctggggcggtacaaaagggcctctggaaccttgcaacaggcaaaggaattctgctgtaaggtgaggaagctgggaagccaatatcttagcctctataagtgtagacattctgtttagtaaaataattttataatatctggaacagccaggagctatccattttgggggcgatatctcttgcctgttctatcattcattacatgcactcagttgaaacaacaattttaggcttctggagcccaggtctcctctatcaggactaattctgagtgccaagatcaatgaccaacatcagaggtagtggagtggttaggtgcatggcctttctgtctggcagattgagtttatgtcttggttatgtcatttcctagctgggtgaccttggtaaagtcacttaacctctctgagtcttcaatcacttgtgaaatgatgataatactactggctaccaaccatccttttcttggggttgggctactgtccaatgagcacgtagtgagggcagtgctgctaacacctacacaaaattcctgcatcagctacagctttactttaccttgccacagtttctggaaaaaaggaaagcctcttttccacaaaaaagggggtaaaaaaacaagaataacatcagctacctttgttgcgttaattttgtagattaagtgaaataaacatggaaaacccttggcacagctctagacacatagaaaagtgctaagaaaagtaattatgcatcacataataacatttcagtcaaagaaggatcacatatatgaccagtggtcctatattgttataatattgtatttttgctatatcttttctgtttagatatacaaaccattgtgttgtaattgcctacagtattcagtacaggaacatgctgtacaggtttgcagcctaggagcaataggctataccataaagcctagaagcgtagtaggctataccatctaggtttgtgtgggtacaccatatatgtttgcacaatgatatagctgcctaaagatgaatttctcagaatgtatccctgttgttaagtgatacatggatgtcattgttattatcatcattcatcttctcactttatggtgggcctccgtaaaactgaccaaggaatatactgcacctgaatcacttcttaccttttctctcttgctcttgccctggacctgaacgcagaatgtcagggagaagtaggaatgtggagtactccaggtgtcagggtactcccagctgacctccacctgccgagaattctttaatggcttcagctgcaagttcttgggtgggtcaggtttgactgtggaagaggataaacatgctttattttcctaataaggctttggagttcagtggttttggcttatgatctcacttgcacctttgatcagctgtgagtccactggatagttacttaacttctgtgagcacctggctggcaaatgtgaggcacctctgccgcaggtgctcacgctggtgcctcgggaaggcagtcattatcaatggccacatctcagatccacatgtattgcaggatctgagagatgctcagaatcctcatcactgaacaatcagatgggaccgcacagtgagaagacctctgctttaatggttatgggccatgcattgaaggaccaccctgtctgtgctaatccctcactttgcactgaacatggaactaagctgagcctctccctggggatgagatgatagattttctatttactgccctttcttttgtcttttcatagcttttggtgcggacatgtcttggagcagttacagtcaattgtctctatgctcaatttgcttgtttattccatgaatactgagcgcccattatgttccagccactggactgtgcatgaaggatacagcagtgagtttcacaaagactccttcctcaattaggtcttctagataagaggaggcagagacatgctagggtgtgtggttgggctctgtaggcctggcactgctccacacaaagatcacggctcctagcaggtgcttcgcacatatgcttccctctgctcttaacacaactgacatctttccttttggtgtcagctaaatgtcacctcctcagagaggccttccttggccaccctatgtgaactagaccccacccaccaagtacaaaccctccataagttttgaagcaagttctctggttctattacactttatcatgctgcctggtcaatgaataaatagtaaaggaaatgacttatggtgaaatccctatctgccaattaaaaaaaaagatgtccattttggaagggtttatattctaagaaagaagtcccttttcaaatgctaaggaaggattggttgggaataattgccattagtaaggaagccctggttctgaatatgaatattatgcttgattaggctctgggattcatcatggaaactatctggcagctgaatcaaagtgcagtaaagagcagctgtagtggagtggatctggatttgaacggctagctgtaagatctgagatctggggcaagttgcttaacttttctaagactcaatttcctcttccttaaaatgcagataacagtatctacatcgtattttgtttttggatgcagaataaaagagatgatgcttgtcaaccacctaccccacagtgcatggggcattgtgaacacgtgacaaatagtatctttccttatggagcacatataatcatccaaaactcactgatgtccctgatgaagaagctgctggtgtagttttcatacttgagcttgtgaacggcatccaccatgacctcaatgggcagactctcctcagcagctgggcaggcactgtcctcctggcactccactgagtactcatactccttgttgtcccctctgactctctctgcagagagtgtagcagctccgcacgtcaccccttgggggtcagaagagctgaagtcaaagacagaaattagcctgtgttacacattggggagagagttcctagtgattgtagccagtaaggcaggtaaggcctcaactgttgtctgaggacacagtttctccaactgggctgatttctacccagagggtaagaaactgccctccccaggagaaaaagcttaaattcaagagcacttacatgtctctggactaaatgaatatggaagtttttgtttgttttagatctagaatctctgattattaaacccccttgttaaaaacttaaattcattttttcccattttaattataaaatcaatacacaaatcagtacacactaattataaaagtaaagatgataaagatccatataaaggaaagaagtgtatgtctccttcatccttaactgctctccacaggtttaagtactaataatagcttgttgtatccttccagaccttcctttttctttttctttctctctctctctctctttttttttttaagagacagggttttgctctgccacccaggctggagtgaagtggtggaatcagaccccactgtagcctcaaactcctaggctcaagtgatcccctccctctgcctcccaagtagctgggactacaggtgtgtgccaccacacccacctaatttttttatttttaatattttctgagatggggtctcactgtgttgcctaggctggtcttgaactcctggcctcaagcaatcattctgtctcagcctcccaaagagttaggatttcaggtgtgagccactgtgcctggccagaactttttcaatgaatattcaagataattgtatacacattttatatatatatatatatatacacacacacacacacacacatatgtatacacacattatatatataatccatgttatatacatctctacattatatatatccactatatatattttacttatacatatagattttatttttatgaactaggatcaaattgtatacatatgattatgtaaccatccttttcccctctgaacatattatggggagctctccatgacaaaacatatgggttgggttatccacttcaatgactgcacattaagcaagagtatagtgtaccatgttttatttaaccattcctctgctgattatgtctttatgcacttggagaaacatttctttagtaagcattttccttttaaagatgaaaaagtgagaccccaatgcttaatttactcagtgaaataatggtaaagtcaggatgatcacctggggtttgcttcggtgatgattaaagtaagccacatgggggttaacacataggtcttgtatttatggaagttgctttcttacggaaagtccaggtgcattggaccaacagcgaacttcagattagtggtgtcagcagaagtaaaaggagttgaggtggccctgggaaaatttcgggaggcctcatcgagttttggagtctgcttcagggcccctaagatctacgccctggagctcttgtttttatttttgactcaaggtgcaatttcagcaagtcatttgtagctttgaattctccgtttatccctttctttggtgctatgaggcttcaggaagcatggccaggcaatttggatgagtgggttcaaacacagcagagactattctcagttcccaataatatcctgccccaacacatacatttattcaagcaatcggctaaaatctccatgttctttcttcaatgtagacaaggaagggcggttgaaaaaacacgggtttgactttgcttttcccattatcattaactgataggtcactgagaggttgcccttaatttcttaatgaaatagttctagaaaaatgctgagaaaccagagcagtttcactcaccctctgctgcttttgacactgaatgtcaaatcagtactgattgtcgtcagccaccagcaggtgaaacgtccagaataattcttggcctcgcatcttagaaaggtcttatttttgggttctggatttgaaaaaaacaaaaattcaatatttaggagttttttgcagaaaggttttgattgtgatgaatcacagatcaccagatggtacagggtgaagttcttccatgcgttgcctcttgggtagctccataaggattgggagtatcccagtggggtgttgattctgcagagtacactacacacctaacatgcctggcagaggcattcattttttgaaggctatcaccccagtgcagagtgatacactatcggttaatttccattagccattaaataatatcaccattatttaaagaaaacatgaggctaagcattcagactgatcatgaaactcccttaatatgagattttgatggttgataacccaaagggtccaggaaagcaaaggaaaatggaggttaacatcaattaacatcaataagagacttgatgttaattcattacactcaccatgacttggcttttcaatttgttgttgttgttgtttttaactcttatgagcgaaagagaaaattgatactatccaagggtatagaattacctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaactggaatgcacataaagtggagaaccaggctgtaagctccaggaactctgggactgtgttttattaacccttcgggcatccccattgcctaaacacaaccttgtttacaacaagtatttggcaaatgcttgctgagatgatctcaaggcatgagaccctgcttgggctcaaggtcatggaaaagagaaacaaagagttttatagctgccatcgactcattgactggaaggctgcctttaatagtaacctttgattatttagcagattggaaacaccttaatataccaaaaactgcaaacagcacaagactctttgccaaaggtctgggggagagaaacttccagcacaatttcagtttcatagagaatacggcagggcacaatattcagcagagtaacatagtggttaaaagctcagggtgtcgagaacaacgaaccaagactgtcatcctgtctccactaaccagctgggggatttggaacaaggtatttcattatcatgagcctcagtttcctcatctgtaaaatgataataataacagtatctgccttacatttgactgaggattaaatgaaaaaaaaaaaaagcacgtaaagtacttagcacagtgtctgccacacagtaaattcggtgttagttatcgttacttatagactgaggagtcagccaactgtacagagaaactctcttaacaattttccatggatatttaaggatttcgttccctctgttttaaatcaccagtggagattttcattctctctgcattattattattattattattatttttagctatcttacactcttatgaagcagtccagtagagcttagtcttcccatttaatgaagaagcgtactgaggccaacgatctaagcatggtcacagcaagtcagaagtacaagggctacagctcagaccttttgtctcttgggctttgcaagggatgcctaatgctagtgtctaaactggcctttgaggaatggcttagtatagtatttcagagtgtgtcatagcaaagcttcattcatttttttaatccatgcataaattattaattgaatagaattgtttaatgtagtagttcccaaagcatgatcgatgaaacaccatcagcagcatcaccttggaatagatcagaaatgcaaattctcagctgctgaatcagaaaccttgggatggatttagttatatggttcttaagctctccagatggtgctgatgcctgctcaagtttatgagtcacagtggttgagagcctgcattttggagtggcagaggcctggatttagaagcagctctagcccttgctccagtcttgtggcctttggcaagacattcaactactgtaagcctcagtgtcatcatctttaaaatgagaaaaatattagcccttgctttagaaggtggatatgtggattatataagctcatacacagtgcccttacatgctttataaatggtggttctcgttattggcctctggatgtaaagcattgtagaatataaagttttcactttacatcaattggattataaggactgaatggataacaaaaataagaggtcaaaagcatttggggtccaactctggggaaatgattagctgtgctgatgtaaaaccatgtggcacttttggctttttcttggttttttttttgtccccctgacacctgaagagcatggttactttcctgtcccaactgaggctcggaagccctagaagatgtttgctcaacagcagggaaaacagctgcaggaggaaagctgaagaggagcatcaagaacctggcacttagcctcactgcatcgtctgccctcctaagtcacacctcggagagtttctcacactgattttctggaatttctgtggtttaccaaatcagaagtcactaaggggtcaaatatgccaggaccaaggttcctgagctcgctaacaatcccagggttggaagggatctgccatatacccctggctcaggcctttgcctttacttaagagagtgtctaaatctctcaaattctcatggagaaggggtggctggattctgtcttcctaaaattcttgagaagagaggagtggtcccagatttctttcaggtacccatttcaagatggtactttctagaatatcaaaagattatttctgatcatcagctgaaatattttcatccttaatttatgtccatttcctcttattcagctctccaaagacctgggaaaaaattacttcatgtccttgtaaaaacgttttcacaaatttaagtgcaccagctttgaagcaagacagaattgggatcaagtccctgttccacccttaccaggtatgtgacattgggcaagttacttaatctctctggcctcagtttccaacatctgtaaaatgggatacagacagtctctacttcatatggctcttgtgagaattaagtgagacagtgcactcaataaacattaactattattattatacaattaaagatagtaatcgccaacattatgatgttggcttcagtgataaaaatctgagggtctttcaggtacagccaacggtcagaatgcacatttactgagtgtgagatactagaaggatgggcagaactccttattttaccattaactattctattagtctactaataatttttactgatcattgggataataaaaagtatctcttgaatcatgaaagatattttaaacaaccaaaatagtaatactagctgacatttactttcactttactatctgtcgagttgtattctgcattcattaacccactaaataacccacgtggtaggcatccactttgtaacatccagtttacagttgaggaaattcaggattagaggtgacccagcttaaagtcacataaccagtcattgacagacctaggacttgaacccaagtcaatgtgagtctgaatcctatgcttcctccaaagtgcttggtcagaggtaagtcatcacagtgccaactgagggggcagtatcatgtggtggaatgagaaacgaacacagttgttatactttgcaaatcctctactttggaattttaattgtgtgaaacagtttttagtaatattggaaaatatcatatgtcatatgtctttccccaccatatcatatatcatatgtctttccccaccatagtgtaccaatgtcatccaatgtgaaaaccaaacaatatagataccatcaaagtagaaaagagagatatggcattgcattttcccagagtgatggctttctctagaggacccagtgcatggttgcccatttcagtttgatttcccaccagtggctgcccaagagtcctggcttagaagtggggcctccacagagaataatgagaggctggttaccatctttcttcagttcccatatggccacgaggggagatgccagaaaaaccagggaaaaccaagagatgaccaactgctggtgacacatctataagaagggagagaagcagggggaagagaaggaggaaaagagaaggaagaggaagaaaaaagagaagaaagaatgtcaataattcttgttacctttgtgaaccatatacacattctagctactttttttttaaataaaatcttgattctcccctttcttctccatactgtcaagcaaatactaagtaaattacaggagttctatggcagtctgtggggaagaacatctctaaatagagaaccatagggtatttggggctctggagctgaaagagacctaaggcaagccatctgatacaatcctcttatttttaagatgagaaacttaaagcttagagaaggaatgtgactttctggatcaacatctagcagttgtttatttagtgcttactacataaagagcactgggctagaagcagttgagagagaaaaaaagggcttacctggatcccgcttcctaggagcaaatacttttactcaataaatatttattaagtcagtgtgttaaatgcctccccgccagctggggagatgagccatacatttgtatcactgcagcacaaagcagtgtgtgctggagcacccagaactgaaggacttgggttagggacaggaacggtaatacagaggcgaactttcaggttctggcaacgacctggtcaccagcccttgctgtaggggtttagcttctcttgttttccaagttcaaagactactctctcccatatagagaacctagtggttctaaaatttgagtgactgtcaggataacctggaagcactgctacaacagacggctgagtcccacccccagagtgtctgattcagcaggcatgagggcctgagaatatgcatttctagaaagtttccaggggaagcagatgctgctggcgctaagaccacaccttgagaaccactggtttaacccatgttcagggtccagaacccctgaaaatcaaagtgttcattgggaaaagaccacaacatattgatatatccaccatgaaatagtaaaatgcataattttatgttaaataagccaccatctgatatagaaaccagtctaggttagtatccagggtttatgaaaatcttccgattgcaaaaattacatgccacttacgaatgaggtagggatgattattgaaacattctgaaacaactccattcaccttgcagagcacccatggtctggaggaaagtggttctgaaaccactgttttaaactatatgaagggcaatgctcaactgtttcagtcaaataccttaaaaatgagcattcctgggttgggtgacggaatattgacaaattacagctttgtcagaactgctactaactctaggcggaccttgctatgtactttattcccttataaagtttgtgagtggcagagacaggcctagaagtcaagccttcttggacactgctcagtgctgtcaccacagcatggagttttaggggcatggctatcacttcagtctaggttatgactgtagcaaagtactagctggcacagggcaccggttagtacattcacaggcttgaagttaaacagacacaggcttgaattttggtcccaccgctcatttgctgttgagcagtgggagcaacttgttggccaagttactcgctgagcctcagtctctttgtctataaaatggacctaatacttatctcaaaggcttgttgggaaaggcaatgagataacatattatagaaggcaaccaataacatattaacttgaacctagaggaagaggtaagggaacaattcggtatctgtgttttatatatcatggatagtcaagaaagtcattctaggcagtagggaaatgtgcacagaggtactgcaattacatgcgcctgtaaaacccagaggctggcacactggaagggaaatagtggagattcctggcatctcctggccacttacgctgaggtattcccactagctgtgtgcccagcacttcctctgcatgcctcagatgcatttgacaatctcaggtgaactgcacttcagggtcaagggaaccccggccatggttctaagaagcaactcccattttagtatcacctacatttgaaaccacagagcactgtccaggagaggtgatggtggtgggtctcctcctttggctctctggcccatcagctgatactagggagcgctatccctcagcccacatttctcagcatggtaagtctgagagtcttgatgattaaaacacatttttgtgtgtgcaatttcataggttttctaaactcttgaaacccttgcatgaaggacaaagggaccccacgttacacactcctacttcctggagtatgctaagggcttcccaacactggtgccaacatcagcatgccaatgacgcttgggtgggcatctcttttttaagttttgtttgttacctttaattagaaaagggagttattattaaagaataaaaagatatagtcacagatggcatgggtggcacatgcaaagatccctagtttgagggccaggcagacttctagaactgtgtgactctgggtgagtcacttggcccctttaagccccacactcctgatctataaaatgggcatgctactgtggtaaggagttagtgacataattcacgtgaagtgtcatggtgcctagatgaagtcaggacccactatttgctcccctccttccacagcatttactacaatggcaacaatacagtctcttctttactgtttgtcttctctgccacactgagtgaactctcttatgccatggatcatgtctatcttgttttgcactttacccccagagtccagcacagagggattccatgacttattagtagcatgaaggaatgtacggaaatcatacaatttcagattccagtaaactaaactctaagaggttacccacattccatctagtatcttgcaattccttgtttgaaaaagtgggtcatcacctggggtttagtaatccctttgttaaaatgacattgttgttgcacacaattttacctgggcaagtaaaaaaacctttttttcccccttctcacagatttaaaaactctttaggaagcaataatagcttttcattttttaactggggccaaagttagttaatccacaagaatggggatcccagctgtcattttggttgatatcacaactgacgaccaagaccatcacaaatatgggagcaagtctgatttgtaacattattataattatgaatccaattactttaaggaatgcacgaaaggctttttaaaaatttcaatagtaaggcaggcaccactcactttagctctatacaatcctttaactgatgggcattaacatttaggaatatacttttatacatcaatacacaccacttcaaaataagataaaacaaacatttccacaaactatgcatacacacagatcttgctatacagttattattaggtggcttttaaaatggacactgtacatcggtccctagagtactaagttttcactaatgttccagcagatgactcaccagggatgcttccaggaatccaccaactccctcctgccagctgctggggtagcacactaacggtttctacacctggacagctgcaggcccacagggaggggagggaggtaggggcttgggaagtgcttaccttgctctgggcaggacggagagtccaatggccctgaaacagatgttgtttcttct

SEQ ID NO: 18 - DNA, 2364 bp, Homo sapiens
agaagaaacaacatctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaattttatttgtactgacaacttttcaagcaaggctgcaagtacatcagttttatgacaatcaggaagaatgcagtgttctgataccagtgccatcatacacttgtgatggatgggaacgcaagagatacttacatggaaacctgacaatgcaaacctgttgagaagatccaggagaacaagatgctagttcccatgtctgtgaagacttcctggagatggtgttgataaagcaatttagggccacttacacttctaagcaagtttaatctttggatgcctgaattttaaaagggctagaaaaaaatgattgaccagcctgggaaacataacaagaccccgtctctacaaaaaaaatttaaaattagccaggcgtggtggctcatgcttgtggtcccagctgttcaggaggatgaggcaggaggatctcttgagcccaggaggtcaaggctatggtgagccgtgattgtgccactgcataccagcctaggtgacagaatgagaccctgtctcaaaaaaaaaaatgattgaaattaaaattcagctttagcttccatggcagtcctcacccccacctctctaaaagacacaggaggatgacacagaaacaccgtaagtgtctggaaggcaaaaagatcttaagattcaagagagaggacaagtagttatggctaaggacatgaaattgtcagaatggcaggtggcttcttaacagccctgtgagaagcagacagatgcaaagaaaatctggaatccctttctcattagcatgaatgaacctgatacacaattatgaccagaaaatatggctccatgaaggtgctacttttaagtaatgtatgtgcgctctgtaaagtgattacatttgtttcctgtttgtttatttatttatttatttttgcattctgaggctgaactaataaaaactcttctttgtaatcata

SEQ ID NO: 19 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 20 - DNA, 708 bp, Mus musculus
atggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcc

SEQ ID NO: 21 - protein, 236 aa, Mus musculus
MVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 22 - DNA, 1005 bp, Mus musculus
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcc

SEQ ID NO: 23 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 24 - DNA, 8239 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgatatcactagtatttaaataacgttgagacgtccttaatcgtcccgacgctaaacggccgcgcacaccgcagccgcaccccgcgcttatcctccagttcgcgtaggaccggcgggtggttaaccaggtccgcaaagttgcggagctcggtaatcagcggaggggtatggtgggtgtccttgtataccgcaaagaaaaagcagtggattgtgccgctggtctcacaggaggcgcggaccaggtaactccgcacggccacgcaagcggagtccgttttgctggtgtgcatggccgtttcggcctgccaggtggcgttgaggcagtaagggggggccacgtgggttatgtccggggcccgtaagaacaggttggtgaggggggtcgctgtcatagtgcaaaaggggggatgcgcccgggcgggaagcccctaagggcactatgacaccggccttggagcggggacggatttatacgttgggttagttccctccgcccacccaggccgtacgccgggcccacccccgccatctgccgtgacccacgccccgccggccatgagcaaagaaggacaacacgtggggcgatttgtttgaaatgttttgtttttattgtacctaaaacaaggagttgcaatgaaaatatttgccgtgcacgtacgggggggcgacgatgtgactggccgtcaactcgcagacacgactcgaacactcctggcggtgcgtgtctaggatttcgatcaggcccgccatgcaggccccggggaggtagaaatgcatcttctctccgaccccgacaccaagggtcgcgtagtcgatctccgcgacgccacgctcgacgcggttggcgagcctggccagaatgacaaacacgaaggatgcaatgtccttaatgtccgccagacgccgccgcgaacacagttcgtccaggccgcacaggcctcgcgccttcaggtagcactggagaaagggccgcaggcgcgtggcgaggttatccagcaccgccgcggccgtcccgataatggggtcctgggggcgcagcggcaggttgtggtggatgcacatcttgcaccacgccagcgtctcgtcggcggaggccagcgcctcgatgaaattttcttggcgcagcacgcagtcgcgcatggccttggcggtcgatgcggcccgaggattgccggcaaagtgcgatagaggctcgggccgtgggcgaccaaggtttcccaggagacccgtctggtctcggcgtcaaagggccctccttggcccgccagcaccggggcccaggggctattcgcggcgggaaacggctgccccccaaaggggtcgtacatgacctgtgcgctgcggccggatcgatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaaggctagagtacttaatacgactcactataggctagcctcgagatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggcggcggcgggagtggcggcgggggttctggcggaggcctcgctagcggtggctccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctgagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgatctccaggctacacgtggattatcatggtatttttcatttacatatgactatacatttcaaatgggccttgcactcaactcgtttccagtttgcatatgccgttatgcgcgaataatgcctggatgtgacgtcatacgtcaaacaggcgcctctggatctcctgctcgtagtgaagcgccacgagcaccaccccggccaccacggcgatataacacaatcgcattgcgatgcccgacaggatgatggaacaacagcgcccgcagacgcccgacagccccttggatcgccccggggcggcggccttgtctgcgttcttgggggccgggccccgccgcagaatacaatacagctctgtcaggccgatggtggagacaaaacaccaggtggtgatggtcagaaacagggggtatgtgatcgcacatgccccccgggatatgaaagcggtgccgacgatgagacccacggccacaaagcgtagcatcaactcgcagcctacgatgaccccgatggcggggcggtggtacaagaaggtgaccgggtccgtctcaaacaactgaaccaggttttgccgctggaccgacagctcgcagagcaggcgggtaattttcgtgtaggggtactgcaggaacacgctcgatacgatgcggcctgcgtagttcaagaggtaggaggccggggccaccatcttgtgggcgggactcacgacaccaaacatacatcggcgttggtggagggcgacgaacgccagatacaggaaccaccctacgaccaccagacgcacccgtgtgtaccatagggtctccagacagttaactgcctcgtggacgttcatgatccgacgattcgtggcgtcgggtgggacctggaagggcacgaccctacccgcgataagattggcgtagcagatatgggcgtggttgcgccagcccccgttgggggggtgtgtcggggcccccagaaacaatagggtctggttcattttcatccacacgagggcggtgtcgttgttggtgccggtggggcgtaccgcgtaaatacatcggtgcagcggactggcaccgaagacggtgtaccacacgagcacgaggccgtacgccgttatcaagacgacggttgagaggtgctgcagggaacggacggcgagcatggcgtgccggcgtcaatggtaaacagcgtgtgcaggcggttgctgtcgcatttggcggcaaagcactgctgacacaaggacacgcacaggcggttgttggccccgacgctcagcgcgacgaatgtccgcgccgtggcgcgactcgcccggccgtgcttaaagcgcagacacgacaggcaacgttatttaatactagtgatatcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 25 - protein, 60 aa, Artificial Sequence
  Description of Artificial Sequence Synthetic polypeptide
  SITE (1)..(60): This sequence may encompass 1-10 “(Gly)x-Ser” repeating units where x=2-5
GGGGGSGGGGGSGGGGGSGGGGGSGGGGGSGGGGGSGGGGGSGGGGGSGGGGGSGGGGGS

SEQ ID NO: 26 - protein, 50 aa, Artificial Sequence
  Description of Artificial Sequence Synthetic polypeptide
  SITE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS

SEQ ID NO: 27 - protein, 19 aa, Artificial Sequence
  Description of Artificial Sequence Synthetic peptide
GGGGSGGGGSGGGLASGGS

SEQ ID NO: 28 - DNA, 57 bp, Artificial Sequence
  Description of Artificial Sequence Synthetic oligonucleotide
ggcggcggcgggagtggcggcgggggttctggcggaggcctcgctagcggtggctcc

SEQ ID NO: 29 - DNA, 33 bp, Artificial Sequence
  Description of Artificial Sequence Synthetic primer
gcgagtctcgagatgtgtcctcagaagctaacc

SEQ ID NO: 30 - DNA, 33 bp, Artificial Sequence
  Description of Artificial Sequence Synthetic primer
atagaagcggccgctcaggcggagctcagatag